(19) United States
(12) Patent Application Publication
(10) Pub. No.: US 2025/0352618 A1
(43) Pub. Date: Nov. 20, 2025

(54) METHODS OF TREATING BRONCHOPULMONARY DYSPLASIA

(71) Applicant: ImmunityBio, Inc., San Diego, CA (US)

(72) Inventors: Hing C. Wong, San Diego, CA (US); Niraj Shrestha, San Diego, CA (US); Mark Leonard Ormiston, San Diego, CA (US); Declan Joseph Gainer, San Diego, CA (US)

(21) Appl. No.: 19/213,482

(22) Filed: May 20, 2025

Related U.S. Application Data
(60) Provisional application No. 63/649,769, filed on May 20, 2024.

(51) Int. Cl. A61K 38/18; A61K 38/20; A61K 38/36; A61P 11/00

(52) U.S. Cl. CPC A61K 38/1841; A61K 38/2086; A61K 38/36; A61P 11/00

(57) ABSTRACT

Provided herein are multi-chain chimeric polypeptides and use thereof in treating bronchopulmonary dysplasia in a subject.

### CROSS-REFERENCE TO RELATED APPLICATIONS

This application claims the benefit of priority under 35 U.S.C. § 119(●) to U.S. Provisional Application No. 63/649,769, filed May 20, 2024. The entire disclosure is incorporated herein by reference.

### REFERENCE TO A SEQUENCE LISTING

This application contains a Sequence Listing submitted electronically as an XML file and is hereby incorporated by reference in its entirety. Said XML file, created on May 20, 2025, is named “PAT.005393.US002.xml” and is 79,181 bytes in size.

### TECHNICAL FIELD

The present disclosure relates to the field of biotechnology, and more specifically, to multi-chain chimeric polypeptides and methods of treating bronchopulmonary dysplasia.

### BACKGROUND

Tissue factor (TF), a 263 amino acid integral membrane glycoprotein with a molecular weight of ˜46 kDa and the trigger protein of the extrinsic blood coagulation pathway, is the primary initiator of coagulation in vivo. Tissue factor, normally not in contact with circulating blood, initiates the coagulation cascade upon exposure to the circulating coagulation serine protease factors. Vascular damage exposes sub-endothelial cells expressing tissue factor, resulting in the formation of a calcium-dependent, high-affinity complex with pre-existing plasma factor VIIa (FVIIa). Binding of the serine protease FVIIa to tissue factor promotes rapid cleavage of FX to FXa and FIX to FIXa. The proteolytic activity of the resulting FXa and an active membrane surface then inefficiently converts a small amount of prothrombin to thrombin. The thrombin generated by FXa initiates platelet activation and activates minute amounts of the pro-cofactors factor V (FV) and factor VIII (FVIII) to become active cofactors, factor Va (FVa) and factor VIIIa (FVIIIa). FIXa complexes with FVIIIa on the platelet surface forming the intrinsic tenase complex, which results in rapid generation of FXa. FXa complexes with FVa to form the pro-thrombinase complex on the activated platelet surface which results in rapid cleavage of prothrombin to thrombin.

In addition to the tissue factor-FVIIa complex, a recent study showed that the tissue factor-FVIIa-FXa complex can activate FVIII, which would provide additional levels of FVIIIa during the initiation phase. The extrinsic pathway is paramount in initiating coagulation via the activation of limited amounts of thrombin, whereas the intrinsic pathway maintains coagulation by dramatic amplification of the initial signal.

Much of the tissue factor expressed on a cell surface is “encrypted,” which must be “decrypted” for full participation in coagulation. The mechanism of “decryption” of cell-surface tissue factor is still unclear at this time, however, exposure of anionic phospholipids plays a major role in this process. Healthy cells actively sequester anionic phospholipids such as phosphatidyl serine (PS) to the inner leaflet of the plasma membrane. Following cellular damage, activation, or increased levels of cytosolic Ca²⁺, this bilayer asymmetry is lost, resulting in increased PS exposure on the outer leaflet, which increases the specific activity of cell-surface tissue factor-FVIIa complexes. PS exposure is known to decrease the apparent Km for activation of FIX and FX by tissue factor-FVIIa complexes, but additional mechanisms could include conformational rearrangement of tissue factor or tissue factor-FVIIa and subsequent exposure of substrate binding sites.

### SUMMARY

Provided herein are methods of treating bronchopulmonary dysplasia (BPD) in a subject that include administering to the subject a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain and the second target-binding domain each bind specifically to a ligand of TGF-β receptor II (TGF-βRII).

In some embodiments of any of the methods described herein, the first target-binding domain and the soluble tissue factor domain directly abut each other in the first chimeric polypeptide. In some embodiments of any of the methods described herein, the first chimeric polypeptide further comprises a linker sequence between the first target-binding domain and the soluble tissue factor domain in the first chimeric polypeptide.

In some embodiments of any of the methods described herein, the soluble tissue factor domain and the first domain of the pair of affinity domains directly abut each other in the first chimeric polypeptide. In some embodiments of any of the methods described herein, the first chimeric polypeptide further comprises a linker sequence between the soluble tissue factor domain and the first domain of the pair of affinity domains in the first chimeric polypeptide.

In some embodiments of any of the methods described herein, the second domain of the pair of affinity domains and the second target-binding domain directly abut each other in the second chimeric polypeptide. In some embodiments of any of the methods described herein, the second chimeric polypeptide further comprises a linker sequence between the second domain of the pair of affinity domains and the second target-binding domain in the second chimeric polypeptide.

In some embodiments of any of the methods described herein, the first target-binding domain and the second target-binding domain bind specifically to the same antigen. In some embodiments of any of the methods described herein, the first target-binding domain and the second target-binding domain bind specifically to different antigens. In some embodiments of any of the methods described herein, one or both of the first target-binding domain and the second target-binding domain is an antigen-binding domain.

In some embodiments of any of the methods described herein, one or both of the first target-binding domain and the second target-binding domain is a soluble TGF-β receptor II (TGF-βRII).

In some embodiments of any of the methods described herein, the first target-binding domain and the second target-binding domain are a soluble TGF-βRII.

In some embodiments of any of the methods described herein, the first chimeric polypeptide further comprises one or more additional target-binding domain(s). In some embodiments of any of the methods described herein, the second chimeric polypeptide further comprises one or more additional target-binding domains.

In some embodiments of any of the methods described herein, the soluble tissue factor domain is a soluble human tissue factor domain. In some embodiments of any of the methods described herein, the soluble human tissue factor domain comprises a sequence that is at least 80% identical to SEQ ID NO: 1.

In some embodiments of any of the methods described herein, the pair of affinity domains is a sushi domain from an alpha chain of human IL-15 receptor (IL15Rα) and a soluble IL-15. In some embodiments of any of the methods described herein, the pair of affinity domains is selected from the group consisting of: barnase and barnstar, a PKA and an AKAP, adapter/docking tag modules based on mutated RNase I fragments, and SNARE modules based on interactions of the proteins syntaxin, synaptotagmin, synaptobrevin, and SNAP25.

In some embodiments of any of the methods described herein, the first chimeric polypeptide and/or the second chimeric polypeptide further comprises a signal sequence at its N-terminal end.

In some embodiments of any of the methods described herein, the first target-binding domain comprises a first sequence that is at least 80% identical to SEQ ID NO: 2 and a second sequence that is at least 80% identical to SEQ ID NO: 2, wherein the first and second sequence are separated by a linker. In some embodiments of any of the methods described herein, the first target-binding domain comprises a first sequence that is at least 90% identical to SEQ ID NO: 2 and a second sequence that is at least 90% identical to SEQ ID NO: 2. In some embodiments of any of the methods described herein, the first target-binding domain comprises a first sequence of SEQ ID NO: 2 and a second sequence of SEQ ID NO: 2. In some embodiments of any of the methods described herein, the linker comprises a sequence of SEQ ID NO: 5.

In some embodiments of any of the methods described herein, the first target-binding domain comprises a sequence that is at least 80% identical to SEQ ID NO: 6. In some embodiments of any of the methods described herein, the first target-binding domain comprises a sequence that is at least 90% identical to SEQ ID NO: 6. In some embodiments of any of the methods described herein, the first target-binding domain comprises a sequence of SEQ ID NO: 6.

In some embodiments of any of the methods described herein, the first chimeric polypeptide comprises a sequence that is at least 80% identical to SEQ ID NO: 8. In some embodiments of any of the methods described herein, the first chimeric polypeptide comprises a sequence that is at least 90% identical to SEQ ID NO: 8. In some embodiments of any of the methods described herein, the first chimeric polypeptide comprises a sequence of SEQ ID NO: 8.

In some embodiments of any of the methods described herein, the first chimeric polypeptide comprises a sequence of SEQ ID NO: 10. In some embodiments of any of the methods described herein, the first chimeric polypeptide comprises a sequence of SEQ ID NO: 12. In some embodiments of any of the methods described herein, the first chimeric polypeptide comprises a sequence of SEQ ID NO: 14.

In some embodiments of any of the methods described herein, the second target-binding domain comprises a first sequence that is at least 80% identical to SEQ ID NO: 2 and a second sequence that is at least 80% identical to SEQ ID NO: 2, wherein the first and second sequence are separated by a linker. In some embodiments of any of the methods described herein, the second target-binding domain comprises a first sequence that is at least 90% identical to SEQ ID NO: 2 and a second sequence that is at least 90% identical to SEQ ID NO: 2. In some embodiments of any of the methods described herein, the second target-binding domain comprises a first sequence of SEQ ID NO: 2 and a second sequence of SEQ ID NO: 2. In some embodiments of any of the methods described herein, the linker comprises a sequence of SEQ ID NO: 5.

In some embodiments of any of the methods described herein, the second target-binding domain comprises a sequence that is at least 80% identical to SEQ ID NO: 6. In some embodiments of any of the methods described herein, the second target-binding domain comprises a sequence that is at least 90% identical to SEQ ID NO: 6. In some embodiments of any of the methods described herein, the second target-binding domain comprises a sequence of SEQ ID NO: 6.

In some embodiments of any of the methods described herein, the second chimeric polypeptide comprises a sequence that is at least 80% identical to SEQ ID NO: 16. In some embodiments of any of the methods described herein, the second chimeric polypeptide comprises a sequence that is at least 90% identical to SEQ ID NO: 16. In some embodiments of any of the methods described herein, the second chimeric polypeptide comprises a sequence of SEQ ID NO: 16. In some embodiments of any of the methods described herein, the second chimeric polypeptide comprises a sequence of SEQ ID NO: 18.

In some embodiments of any of the methods described herein, the subject has been identified or diagnosed as having bronchopulmonary dysplasia (BPD).

Also provided herein are methods of treating bronchopulmonary dysplasia (BPD) in a subject that include administering to the subject a therapeutically effective amount of a multi-chain chimeric polypeptide, wherein the multi-chain chimeric polypeptide comprises: (a) a first chimeric polypeptide comprising: (i) a first target-binding domain comprising: a first sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; and a second sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; (ii) a soluble tissue factor domain; and (iii) a first domain of a pair of affinity domains; and (b) a second chimeric polypeptide comprising: (i) a second domain of a pair of affinity domains; and (ii) a second target-binding domain comprising: a first sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; and a second sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

In some embodiments of any of the methods described herein, the first sequence of the first target-binding domain comprises an asparagine at amino acid position 32 in SEQ ID NO: 20. In some embodiments of any of the methods described herein, the first sequence of the first target-binding domain comprises an alanine at amino acid position 119 in SEQ ID NO: 20. In some embodiments of any of the methods described herein, the first sequence of the first target-binding domain comprises an asparagine at amino acid position 32 and an alanine at amino acid position 119 in SEQ ID NO: 20.

In some embodiments of any of the methods described herein, the second sequence of the first target-binding domain comprises an asparagine at amino acid position 32 in SEQ ID NO: 20. In some embodiments of any of the methods described herein, the second sequence of the first target-binding domain comprises an alanine at amino acid position 119 in SEQ ID NO: 20. In some embodiments of any of the methods described herein, the second sequence of the first target-binding domain comprises an asparagine at amino acid position 32 and an alanine at amino acid position 119 in SEQ ID NO: 20.

In some embodiments of any of the methods described herein, the first sequence of the second target-binding domain comprises an asparagine at amino acid position 32 in SEQ ID NO: 20. In some embodiments of any of the methods described herein, the first sequence of the second target-binding domain comprises an alanine at amino acid position 119 in SEQ ID NO: 20. In some embodiments of any of the methods described herein, the first sequence of the second target-binding domain comprises an asparagine at amino acid position 32 and an alanine at amino acid position 119 in SEQ ID NO: 20.

In some embodiments of any of the methods described herein, the second sequence of the second target-binding domain comprises an asparagine at amino acid position 32 in SEQ ID NO: 20. In some embodiments of any of the methods described herein, the second sequence of the second target-binding domain comprises an alanine at amino acid position 119 in SEQ ID NO: 20. In some embodiments of any of the methods described herein, the second sequence of the second target-binding domain comprises an asparagine at amino acid position 32 and an alanine at amino acid position 119 in SEQ ID NO: 20.

In some embodiments of any of the methods described herein, the first target-binding domain and the soluble tissue factor domain directly abut each other in the first chimeric polypeptide. In some embodiments of any of the methods described herein, the first chimeric polypeptide further comprises a linker sequence between the first target-binding domain and the soluble tissue factor domain in the first chimeric polypeptide.

In some embodiments of any of the methods described herein, the soluble tissue factor domain and the first domain of the pair of affinity domains directly abut each other in the first chimeric polypeptide. In some embodiments of any of the methods described herein, the first chimeric polypeptide further comprises a linker sequence between the soluble tissue factor domain and the first domain of the pair of affinity domains in the first chimeric polypeptide.

In some embodiments of any of the methods described herein, the second domain of the pair of affinity domains and the second target-binding domain directly abut each other in the second chimeric polypeptide. In some embodiments of any of the methods described herein, the second chimeric polypeptide further comprises a linker sequence between the second domain of the pair of affinity domains and the second target-binding domain in the second chimeric polypeptide.

In some embodiments of any of the methods described herein, the first chimeric polypeptide further comprises one or more additional target-binding domain(s). In some embodiments of any of the methods described herein, the second chimeric polypeptide further comprises one or more additional target-binding domain(s).

In some embodiments of any of the methods described herein, the soluble tissue factor domain is a soluble human tissue factor domain. In some embodiments of any of the methods described herein, the human soluble tissue factor domain does not initiate blood coagulation. In some embodiments of any of the methods described herein, the soluble human tissue factor domain comprises a sequence that is at least 80% identical to SEQ ID NO: 1.

In some embodiments of any of the methods described herein, the pair of affinity domains is a sushi domain from an alpha chain of human IL-15 receptor (IL-15Rα) and a soluble IL-15.

In some embodiments of any of the methods described herein, the first target-binding domain further comprises a linker disposed between the first sequence and the second sequence. In some embodiments of any of the methods described herein, the first sequence in the first target-binding domain is at least 90% identical to SEQ ID NO: 20 and the second sequence in the first target-binding domain is at least 90% identical to SEQ ID NO: 20. In some embodiments of any of the methods described herein, the first sequence in the first target-binding domain is SEQ ID NO: 20 and the second sequence in the first target-binding domain is SEQ ID NO: 20. In some embodiments of any of the methods described herein, the linker comprises a sequence of SEQ ID NO: 5.

In some embodiments of any of the methods described herein, the first target-binding domain comprises a sequence that is at least 80% identical to SEQ ID NO: 25. In some embodiments of any of the methods described herein, the first target-binding domain comprises a sequence that is at least 90% identical to SEQ ID NO: 25. In some embodiments of any of the methods described herein, the first target-binding domain comprises a sequence of SEQ ID NO: 25.

In some embodiments of any of the methods described herein, the first chimeric polypeptide comprises a sequence that is at least 80% identical to SEQ ID NO: 28. In some embodiments of any of the methods described herein, the first chimeric polypeptide comprises a sequence that is at least 90% identical to SEQ ID NO: 28. In some embodiments of any of the methods described herein, the first chimeric polypeptide comprises a sequence of SEQ ID NO: 28. In some embodiments of any of the methods described herein, the first chimeric polypeptide comprises a sequence of SEQ ID NO: 29.

In some embodiments of any of the methods described herein, the second target-binding domain further comprises a linker disposed between the first sequence and the second sequence. In some embodiments of any of the methods described herein, the first sequence in the second target-binding domain is at least 90% identical to SEQ ID NO: 20 and the second sequence in the second target-binding domain is at least 90% identical to SEQ ID NO: 20. In some embodiments of any of the methods described herein, the first sequence in the second target-binding domain is SEQ ID NO: 20 and the second sequence in the second target-binding domain is SEQ ID NO: 20. In some embodiments of any of the methods described herein, the linker comprises a sequence of SEQ ID NO: 5.

In some embodiments of any of the methods described herein, the second target-binding domain comprises a sequence that is at least 80% identical to SEQ ID NO: 25. In some embodiments of any of the methods described herein, the second target-binding domain comprises a sequence that is at least 90% identical to SEQ ID NO: 25. In some embodiments of any of the methods described herein, the second target-binding domain comprises a sequence of SEQ ID NO: 25.

In some embodiments of any of the methods described herein, the second chimeric polypeptide comprises a sequence that is at least 80% identical to SEQ ID NO: 31. In some embodiments of any of the methods described herein, the first chimeric polypeptide comprises a sequence that is at least 80% identical to SEQ ID NO: 28. In some embodiments of any of the methods described herein, the second chimeric polypeptide comprises a sequence that is at least 90% identical to SEQ ID NO: 31. In some embodiments of any of the methods described herein, the second chimeric polypeptide comprises a sequence of SEQ ID NO: 31. In some embodiments of any of the methods described herein, the first chimeric polypeptide comprises a sequence of SEQ ID NO: 28. In some embodiments of any of the methods described herein, the second chimeric polypeptide comprises a sequence of SEQ ID NO: 33.

In some embodiments of any of the methods described herein, the subject has been identified or diagnosed as having bronchopulmonary dysplasia (BPD).

As used herein, the term “chimeric” refers to a polypeptide that includes amino acid sequences (e.g., domains) originally derived from two different sources (e.g., two different naturally-occurring proteins, e.g., from the same or different species). For example, a chimeric polypeptide can include domains from at least two different naturally occurring human proteins. In some examples, a chimeric polypeptide can include a domain that is a synthetic sequence (e.g., an scFv) and a domain that is derived from a naturally-occurring protein (e.g., a naturally-occurring human protein). In some embodiments, a chimeric polypeptide can include at least two different domains that are synthetic sequences (e.g., two different scFvs).

An “antigen-binding domain” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

A “soluble tissue factor domain” refers to a polypeptide having at least 70% identity (e.g., at least 75% identity, at least 80% identity, at least 85% identity, at least 90% identity, at least 95% identity, at least 99% identity, or 100% identical) to a segment of a wildtype mammalian tissue factor protein (e.g., a wildtype human tissue factor protein) that lacks the transmembrane domain and the intracellular domain. Non-limiting examples of soluble tissue factor domains are described herein.

The term “soluble interleukin receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble interleukin receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype interleukin receptor and retains its ability to specifically bind to one or more of its natural ligands, but lacks its transmembrane domain (and optionally, further lacks its intracellular domain). Non-limiting examples of soluble interleukin receptors are described herein.

The term “soluble cytokine receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble cytokine receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype cytokine receptor and retains its ability to specifically bind to one or more of its natural ligands, but lacks its transmembrane domain (and optionally, further lacks its intracellular domain). Non-limiting examples of soluble cytokine receptors are described herein.

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, and multi-specific antibodies. One example of an antigen-binding domain is an antigen-binding domain formed by a VH-VL dimer. Additional examples of an antibody are described herein. Additional examples of an antibody are known in the art.

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an antigen-binding domain and an antigen or epitope. The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining the affinity for an antigen-binding domain and its corresponding antigen or epitope are known in the art.

A “multi-chain polypeptide” as used herein to refers to a polypeptide comprising two or more (e.g., three, four, five, six, seven, eight, nine, or ten) protein chains (e.g., at least a first chimeric polypeptide and a second polypeptide), where the two or more proteins chains associate through non-covalent bonds to form a quaternary structure.

The term “pair of affinity domains” is two different protein domain(s) that bind specifically to each other with a K_{D} of less than of less than 1×10⁻⁷ M (e.g., less than 1×10⁻⁸ M, less than 1×10⁻⁹ M, less than 1×10⁻¹⁰ M, or less than 1×10⁻¹¹ M). In some examples, a pair of affinity domains can be a pair of naturally-occurring proteins. In some embodiments, a pair of affinity domains can be a pair of synthetic proteins. Non-limiting examples of pairs of affinity domains are described herein.

The term “epitope” means a portion of an antigen that specifically binds to an antigen-binding domain. Epitopes can, e.g., consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. Methods for identifying an epitope to which an antigen-binding domain binds are known in the art.

The term “treatment” means to ameliorate at least one symptom of a disorder. Generally, the methods of treatment include administering a therapeutically effective amount of composition that reduces at least one symptom of a disorder to a subject who is in need of, or who has been determined to be in need of such treatment.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows a schematic of the TGFRt15-TGFRs construct.

FIG. 2 shows an additional schematic of the TGFRt15-TGFRs construct.

FIG. 3 shows an exemplary schematic of a proposed model depicting actions of NK cell activity and cellular senescence on postnatal lung patterning.

FIG. 4 is an exemplary schematic of the experimental design using a mouse model.

FIGS. 5A-5F show administration of HCW9218 restores normal heart weight in hyperoxic C57BL/6 mouse pups by postnatal day 7 (P7). FIGS. 5A-5C show body weight (FIG. 5A), heart weight (FIG. 5B), and heart weight relative to body weight (FIG. 5C) of neonates at P3. FIGS. 5D-5F show body weight (FIG. 5D), heart weight (FIG. 5E), and heart weight relative to body weight (FIG. 5F) or neonates at P7. (◯) female mice, (●) male mice. ****p<0.0001, ***p<0.001, **p<0.01, *p<0.05, ns=not significant.

FIGS. 6A-6B show HCW9218 prevents impaired airway patterning in hyperoxic C57BL/6 pups. FIG. 6A shows representative H&E-stained 10 μm lung sections from pups at P7. FIG. 6B shows quantification of mean linear intercept (LM) at P7. Scale bars=100 μm. (o) female mice, (●) male mice. **p<0.01, ns=not significant.

FIGS. 7A-7C show HCW9218 prevents the hyperoxia-induced enlargement of alveolar ducts. FIG. 7A shows representative masked image depicting large respiratory zone airways (alveolar ducts) in distal lung tissue. FIG. 7B shows quantification of mean alveolar duct area at P7. FIG. 7C shows quantification of the number of ducts per 20×HPF. (◯) female mice, (●) male mice. *p<0.05, ns=not significant.

FIGS. 8A-8B show vascular density may be partially restored by the administration of HCW9218. FIG. 8A shows representative P7 lung sections stained with von Willebrand Factor (vWF) and smooth muscle α-actin (aSMA). FIG. 8B shows quantification of vascular density at P3 and P7. (◯) female mice, (●) male mice. ns=not significant.

FIGS. 9A-9B show HCW9218 prevents the accumulation of senescent cells in the lungs of hyperoxic neonates. FIG. 9A shows representative P3 lung sections stained for senescence-associated β-galactosidase (β-gal). FIG. 9B shows quantification of the number of β-Gal⁺ cells per 20×HPF at P3. (◯) female mice, (●) male mice. *p<0.05, ns=not significant.

### DETAILED DESCRIPTION

Provided herein are methods of treating bronchopulmonary dysplasia (BPD) in a subject, the method comprising administering to the subject a therapeutically effective amount of a multi-chain chimeric polypeptide, wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain and the second target-binding domain each bind specifically to a ligand of TGF-β receptor II (TGF-βRII).

Also provided herein are methods of treating bronchopulmonary dysplasia (BPD) in a subject, the method comprising administering to the subject a therapeutically effective amount of a multi-chain chimeric polypeptide, wherein the multi-chain chimeric polypeptide comprises: (a) a first chimeric polypeptide comprising: (i) a first target-binding domain comprising: a first sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; and a second sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; (ii) a soluble tissue factor domain; and (iii) a first domain of a pair of affinity domains; and (b) a second chimeric polypeptide comprising: (i) a second domain of a pair of affinity domains; and (ii) a second target-binding domain comprising: a first sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; and a second sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

In some embodiments, the first sequence of the first target-binding domain comprises an asparagine, a glutamine, a cysteine, or a tyrosine at amino acid position 32 in SEQ ID NO: 20. In some embodiments, the first sequence of the first target-binding domain comprises an asparagine at amino acid position 32 in SEQ ID NO: 20.

In some embodiments, the first sequence of the first target-binding domain comprises an alanine, a glycine, or a valine at amino acid position 119 in SEQ ID NO: 20. In some embodiments, the first sequence of the first target-binding domain comprises an alanine at amino acid position 119 in SEQ ID NO: 20.

In some embodiments, the first sequence of the first target-binding domain comprises an asparagine, a glutamine, a cysteine, or a tyrosine at amino acid position 32 and an alanine, a glycine, or a valine at amino acid position 119 in SEQ ID NO: 20. In some embodiments, the first sequence of the first target-binding domain comprises an asparagine at amino acid position 32 and an alanine at amino acid position 119 in SEQ ID NO: 20.

In some embodiments, the second sequence of the first target-binding domain comprises an asparagine, a glutamine, a cysteine, or a tyrosine at amino acid position 32 in SEQ ID NO: 20.

In some embodiments, the second sequence of the first target-binding domain comprises an asparagine at amino acid position 32 in SEQ ID NO: 20.

In some embodiments, the second sequence of the first target-binding domain comprises an alanine, a glycine, or a valine at amino acid position 119 in SEQ ID NO: 20. In some embodiments, the second sequence of the first target-binding domain comprises an alanine at amino acid position 119 in SEQ ID NO: 20.

In some embodiments, the second sequence of the first target-binding domain comprises an asparagine, a glutamine, a cysteine, or a tyrosine at amino acid position 32 and an alanine, a glycine, or a valine at amino acid position 119 in SEQ ID NO: 20. In some embodiments, the second sequence of the first target-binding domain comprises an asparagine at amino acid position 32 and an alanine at amino acid position 119 in SEQ ID NO: 20.

In some embodiments, the first and second sequence of the first target-binding domain are separated by a linker (e.g., any of the exemplary linkers described herein, e.g., SEQ ID NO: 5). In some embodiments, the first sequence of the second target-binding domain comprises an asparagine, a glutamine, a cysteine, or a tyrosine at amino acid position 32 in SEQ ID NO: 20.

In some embodiments, the first sequence of the second target-binding domain comprises an asparagine at amino acid position 32 in SEQ ID NO: 20.

In some embodiments, the first sequence of the second target-binding domain comprises an alanine, a glycine, or a valine at amino acid position 119 in SEQ ID NO: 20. In some embodiments, the first sequence of the second target-binding domain comprises an alanine at amino acid position 119 in SEQ ID NO: 20.

In some embodiments, the first sequence of the second target-binding domain comprises an asparagine, a glutamine, a cysteine, or a tyrosine at amino acid position 32 and an alanine, a glycine, or a valine at amino acid position 119 in SEQ ID NO: 20. In some embodiments, the first sequence of the second target-binding domain comprises an asparagine at amino acid position 32 and an alanine at amino acid position 119 in SEQ ID NO: 20.

In some embodiments, the second sequence of the second target-binding domain comprises an asparagine, a glutamine, a cysteine, or a tyrosine at amino acid position 32 in SEQ ID NO: 20.

In some embodiments, the second sequence of the second target-binding domain comprises an asparagine at amino acid position 32 in SEQ ID NO: 20.

In some embodiments, the second sequence of the second target-binding domain comprises an alanine, a glycine, or a valine at amino acid position 119 in SEQ ID NO: 20. In some embodiments, the second sequence of the second target-binding domain comprises an alanine at amino acid position 119 in SEQ ID NO: 20.

In some embodiments, the second sequence of the second target-binding domain comprises an asparagine, a glutamine, a cysteine, or a tyrosine at amino acid position 32 and an alanine, a glycine, or a valine at amino acid position 119 in SEQ ID NO: 20. In some embodiments, the second sequence of the second target-binding domain comprises an asparagine at amino acid position 32 and an alanine at amino acid position 119 in SEQ ID NO: 20.

In some embodiments, the first and second sequence of the second target-binding domain are separated by a linker (e.g., any of the exemplary linkers described herein, e.g., SEQ ID NO: 5).

In some examples of any of the multi-chain chimeric polypeptides described herein the total length of first chimeric polypeptide and/or the second chimeric polypeptide can each independently be about 50 amino acids to about 3000 amino acids, about 50 amino acids to about 2500 amino acids, about 50 amino acids to about 2000 amino acids, about 50 amino acids to about 1500 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 480 amino acids, about 50 amino acids to about 460 amino acids, about 50 amino acids to about 440 amino acids, about 50 amino acids to about 420 amino acids, about 50 amino acids to about 400 amino acids, about 50 amino acids to about 380 amino acids, about 50 amino acids to about 360 amino acids, about 50 amino acids to about 340 amino acids, about 50 amino acids to about 320 amino acids, about 50 amino acids to about 300 amino acids, about 50 amino acids to about 280 amino acids, about 50 amino acids to about 260 amino acids, about 50 amino acids to about 240 amino acids, about 50 amino acids to about 220 amino acids, about 50 amino acids to about 200 amino acids, about 50 amino acids to about 150 amino acids, about 50 amino acids to about 100 amino acids, about 100 amino acids to about 3000 amino acids, about 100 amino acids to about 2500 amino acids, about 100 amino acids to about 2000 amino acids, about 100 amino acids to about 1500 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 480 amino acids, about 100 amino acids to about 460 amino acids, about 100 amino acids to about 440 amino acids, about 100 amino acids to about 420 amino acids, about 100 amino acids to about 400 amino acids, about 100 amino acids to about 380 amino acids, about 100 amino acids to about 360 amino acids, about 100 amino acids to about 340 amino acids, about 100 amino acids to about 320 amino acids, about 100 amino acids to about 300 amino acids, about 100 amino acids to about 280 amino acids, about 100 amino acids to about 260 amino acids, about 100 amino acids to about 240 amino acids, about 100 amino acids to about 220 amino acids, about 100 amino acids to about 200 amino acids, about 100 amino acids to about 150 amino acids, about 150 amino acids to about 3000 amino acids, about 150 amino acids to about 2500 amino acids, about 150 amino acids to about 2000 amino acids, about 150 amino acids to about 1500 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 480 amino acids, about 150 amino acids to about 460 amino acids, about 150 amino acids to about 440 amino acids, about 150 amino acids to about 420 amino acids, about 150 amino acids to about 400 amino acids, about 150 amino acids to about 380 amino acids, about 150 amino acids to about 360 amino acids, about 150 amino acids to about 340 amino acids, about 150 amino acids to about 320 amino acids, about 150 amino acids to about 300 amino acids, about 150 amino acids to about 280 amino acids, about 150 amino acids to about 260 amino acids, about 150 amino acids to about 240 amino acids, about 150 amino acids to about 220 amino acids, about 150 amino acids to about 200 amino acids, about 200 amino acids to about 3000 amino acids, about 200 amino acids to about 2500 amino acids, about 200 amino acids to about 2000 amino acids, about 200 amino acids to about 1500 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 480 amino acids, about 200 amino acids to about 460 amino acids, about 200 amino acids to about 440 amino acids, about 200 amino acids to about 420 amino acids, about 200 amino acids to about 400 amino acids, about 200 amino acids to about 380 amino acids, about 200 amino acids to about 360 amino acids, about 200 amino acids to about 340 amino acids, about 200 amino acids to about 320 amino acids, about 200 amino acids to about 300 amino acids, about 200 amino acids to about 280 amino acids, about 200 amino acids to about 260 amino acids, about 200 amino acids to about 240 amino acids, about 200 amino acids to about 220 amino acids, about 220 amino acids to about 3000 amino acids, about 220 amino acids to about 2500 amino acids, about 220 amino acids to about 2000 amino acids, about 220 amino acids to about 1500 amino acids, about 220 amino acids to about 1000 amino acids, about 220 amino acids to about 950 amino acids, about 220 amino acids to about 900 amino acids, about 220 amino acids to about 850 amino acids, about 220 amino acids to about 800 amino acids, about 220 amino acids to about 750 amino acids, about 220 amino acids to about 700 amino acids, about 220 amino acids to about 650 amino acids, about 220 amino acids to about 600 amino acids, about 220 amino acids to about 550 amino acids, about 220 amino acids to about 500 amino acids, about 220 amino acids to about 480 amino acids, about 220 amino acids to about 460 amino acids, about 220 amino acids to about 440 amino acids, about 220 amino acids to about 420 amino acids, about 220 amino acids to about 400 amino acids, about 220 amino acids to about 380 amino acids, about 220 amino acids to about 360 amino acids, about 220 amino acids to about 340 amino acids, about 220 amino acids to about 320 amino acids, about 220 amino acids to about 300 amino acids, about 220 amino acids to about 280 amino acids, about 220 amino acids to about 260 amino acids, about 220 amino acids to about 240 amino acids, about 240 amino acids to about 3000 amino acids, about 240 amino acids to about 2500 amino acids, about 240 amino acids to about 2000 amino acids, about 240 amino acids to about 1500 amino acids, about 240 amino acids to about 1000 amino acids, about 240 amino acids to about 950 amino acids, about 240 amino acids to about 900 amino acids, about 240 amino acids to about 850 amino acids, about 240 amino acids to about 800 amino acids, about 240 amino acids to about 750 amino acids, about 240 amino acids to about 700 amino acids, about 240 amino acids to about 650 amino acids, about 240 amino acids to about 600 amino acids, about 240 amino acids to about 550 amino acids, about 240 amino acids to about 500 amino acids, about 240 amino acids to about 480 amino acids, about 240 amino acids to about 460 amino acids, about 240 amino acids to about 440 amino acids, about 240 amino acids to about 420 amino acids, about 240 amino acids to about 400 amino acids, about 240 amino acids to about 380 amino acids, about 240 amino acids to about 360 amino acids, about 240 amino acids to about 340 amino acids, about 240 amino acids to about 320 amino acids, about 240 amino acids to about 300 amino acids, about 240 amino acids to about 280 amino acids, about 240 amino acids to about 260 amino acids, about 260 amino acids to about 3000 amino acids, about 260 amino acids to about 2500 amino acids, about 260 amino acids to about 2000 amino acids, about 260 amino acids to about 1500 amino acids, about 260 amino acids to about 1000 amino acids, about 260 amino acids to about 950 amino acids, about 260 amino acids to about 900 amino acids, about 260 amino acids to about 850 amino acids, about 260 amino acids to about 800 amino acids, about 260 amino acids to about 750 amino acids, about 260 amino acids to about 700 amino acids, about 260 amino acids to about 650 amino acids, about 260 amino acids to about 600 amino acids, about 260 amino acids to about 550 amino acids, about 260 amino acids to about 500 amino acids, about 260 amino acids to about 480 amino acids, about 260 amino acids to about 460 amino acids, about 260 amino acids to about 440 amino acids, about 260 amino acids to about 420 amino acids, about 260 amino acids to about 400 amino acids, about 260 amino acids to about 380 amino acids, about 260 amino acids to about 360 amino acids, about 260 amino acids to about 340 amino acids, about 260 amino acids to about 320 amino acids, about 260 amino acids to about 300 amino acids, about 260 amino acids to about 280 amino acids, about 280 amino acids to about 3000 amino acids, about 280 amino acids to about 2500 amino acids, about 280 amino acids to about 2000 amino acids, about 280 amino acids to about 1500 amino acids, about 280 amino acids to about 1000 amino acids, about 280 amino acids to about 950 amino acids, about 280 amino acids to about 900 amino acids, about 280 amino acids to about 850 amino acids, about 280 amino acids to about 800 amino acids, about 280 amino acids to about 750 amino acids, about 280 amino acids to about 700 amino acids, about 280 amino acids to about 650 amino acids, about 280 amino acids to about 600 amino acids, about 280 amino acids to about 550 amino acids, about 280 amino acids to about 500 amino acids, about 280 amino acids to about 480 amino acids, about 280 amino acids to about 460 amino acids, about 280 amino acids to about 440 amino acids, about 280 amino acids to about 420 amino acids, about 280 amino acids to about 400 amino acids, about 280 amino acids to about 380 amino acids, about 280 amino acids to about 360 amino acids, about 280 amino acids to about 340 amino acids, about 280 amino acids to about 320 amino acids, about 280 amino acids to about 300 amino acids, about 300 amino acids to about 3000 amino acids, about 300 amino acids to about 2500 amino acids, about 300 amino acids to about 2000 amino acids, about 300 amino acids to about 1500 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 480 amino acids, about 300 amino acids to about 460 amino acids, about 300 amino acids to about 440 amino acids, about 300 amino acids to about 420 amino acids, about 300 amino acids to about 400 amino acids, about 300 amino acids to about 380 amino acids, about 300 amino acids to about 360 amino acids, about 300 amino acids to about 340 amino acids, about 300 amino acids to about 320 amino acids, about 320 amino acids to about 3000 amino acids, about 320 amino acids to about 2500 amino acids, about 320 amino acids to about 2000 amino acids, about 320 amino acids to about 1500 amino acids, about 320 amino acids to about 1000 amino acids, about 320 amino acids to about 950 amino acids, about 320 amino acids to about 900 amino acids, about 320 amino acids to about 850 amino acids, about 320 amino acids to about 800 amino acids, about 320 amino acids to about 750 amino acids, about 320 amino acids to about 700 amino acids, about 320 amino acids to about 650 amino acids, about 320 amino acids to about 600 amino acids, about 320 amino acids to about 550 amino acids, about 320 amino acids to about 500 amino acids, about 320 amino acids to about 480 amino acids, about 320 amino acids to about 460 amino acids, about 320 amino acids to about 440 amino acids, about 320 amino acids to about 420 amino acids, about 320 amino acids to about 400 amino acids, about 320 amino acids to about 380 amino acids, about 320 amino acids to about 360 amino acids, about 320 amino acids to about 340 amino acids, about 340 amino acids to about 3000 amino acids, about 340 amino acids to about 2500 amino acids, about 340 amino acids to about 2000 amino acids, about 340 amino acids to about 1500 amino acids, about 340 amino acids to about 1000 amino acids, about 340 amino acids to about 950 amino acids, about 340 amino acids to about 900 amino acids, about 340 amino acids to about 850 amino acids, about 340 amino acids to about 800 amino acids, about 340 amino acids to about 750 amino acids, about 340 amino acids to about 700 amino acids, about 340 amino acids to about 650 amino acids, about 340 amino acids to about 600 amino acids, about 340 amino acids to about 550 amino acids, about 340 amino acids to about 500 amino acids, about 340 amino acids to about 480 amino acids, about 340 amino acids to about 460 amino acids, about 340 amino acids to about 440 amino acids, about 340 amino acids to about 420 amino acids, about 340 amino acids to about 400 amino acids, about 340 amino acids to about 380 amino acids, about 340 amino acids to about 360 amino acids, about 360 amino acids to about 3000 amino acids, about 360 amino acids to about 2500 amino acids, about 360 amino acids to about 2000 amino acids, about 360 amino acids to about 1500 amino acids, about 360 amino acids to about 1000 amino acids, about 360 amino acids to about 950 amino acids, about 360 amino acids to about 900 amino acids, about 360 amino acids to about 850 amino acids, about 360 amino acids to about 800 amino acids, about 360 amino acids to about 750 amino acids, about 360 amino acids to about 700 amino acids, about 360 amino acids to about 650 amino acids, about 360 amino acids to about 600 amino acids, about 360 amino acids to about 550 amino acids, about 360 amino acids to about 500 amino acids, about 360 amino acids to about 480 amino acids, about 360 amino acids to about 460 amino acids, about 360 amino acids to about 440 amino acids, about 360 amino acids to about 420 amino acids, about 360 amino acids to about 400 amino acids, about 360 amino acids to about 380 amino acids, about 380 amino acids to about 3000 amino acids, about 380 amino acids to about 2500 amino acids, about 380 amino acids to about 2000 amino acids, about 380 amino acids to about 1500 amino acids, about 380 amino acids to about 1000 amino acids, about 380 amino acids to about 950 amino acids, about 380 amino acids to about 900 amino acids, about 380 amino acids to about 850 amino acids, about 380 amino acids to about 800 amino acids, about 380 amino acids to about 750 amino acids, about 380 amino acids to about 700 amino acids, about 380 amino acids to about 650 amino acids, about 380 amino acids to about 600 amino acids, about 380 amino acids to about 550 amino acids, about 380 amino acids to about 500 amino acids, about 380 amino acids to about 480 amino acids, about 380 amino acids to about 460 amino acids, about 380 amino acids to about 440 amino acids, about 380 amino acids to about 420 amino acids, about 380 amino acids to about 400 amino acids, about 400 amino acids to about 3000 amino acids, about 400 amino acids to about 2500 amino acids, about 400 amino acids to about 2000 amino acids, about 400 amino acids to about 1500 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 480 amino acids, about 400 amino acids to about 460 amino acids, about 400 amino acids to about 440 amino acids, about 400 amino acids to about 420 amino acids, about 420 amino acids to about 3000 amino acids, about 420 amino acids to about 2500 amino acids, about 420 amino acids to about 2000 amino acids, about 420 amino acids to about 1500 amino acids, about 420 amino acids to about 1000 amino acids, about 420 amino acids to about 950 amino acids, about 420 amino acids to about 900 amino acids, about 420 amino acids to about 850 amino acids, about 420 amino acids to about 800 amino acids, about 420 amino acids to about 750 amino acids, about 420 amino acids to about 700 amino acids, about 420 amino acids to about 650 amino acids, about 420 amino acids to about 600 amino acids, about 420 amino acids to about 550 amino acids, about 420 amino acids to about 500 amino acids, about 420 amino acids to about 480 amino acids, about 420 amino acids to about 460 amino acids, about 420 amino acids to about 440 amino acids, about 440 amino acids to about 3000 amino acids, about 440 amino acids to about 2500 amino acids, about 440 amino acids to about 2000 amino acids, about 440 amino acids to about 1500 amino acids, about 440 amino acids to about 1000 amino acids, about 440 amino acids to about 950 amino acids, about 440 amino acids to about 900 amino acids, about 440 amino acids to about 850 amino acids, about 440 amino acids to about 800 amino acids, about 440 amino acids to about 750 amino acids, about 440 amino acids to about 700 amino acids, about 440 amino acids to about 650 amino acids, about 440 amino acids to about 600 amino acids, about 440 amino acids to about 550 amino acids, about 440 amino acids to about 500 amino acids, about 440 amino acids to about 480 amino acids, about 440 amino acids to about 460 amino acids, about 460 amino acids to about 3000 amino acids, about 460 amino acids to about 2500 amino acids, about 460 amino acids to about 2000 amino acids, about 460 amino acids to about 1500 amino acids, about 460 amino acids to about 1000 amino acids, about 460 amino acids to about 950 amino acids, about 460 amino acids to about 900 amino acids, about 460 amino acids to about 850 amino acids, about 460 amino acids to about 800 amino acids, about 460 amino acids to about 750 amino acids, about 460 amino acids to about 700 amino acids, about 460 amino acids to about 650 amino acids, about 460 amino acids to about 600 amino acids, about 460 amino acids to about 550 amino acids, about 460 amino acids to about 500 amino acids, about 460 amino acids to about 480 amino acids, about 480 amino acids to about 3000 amino acids, about 480 amino acids to about 2500 amino acids, about 480 amino acids to about 2000 amino acids, about 480 amino acids to about 1500 amino acids, about 480 amino acids to about 1000 amino acids, about 480 amino acids to about 950 amino acids, about 480 amino acids to about 900 amino acids, about 480 amino acids to about 850 amino acids, about 480 amino acids to about 800 amino acids, about 480 amino acids to about 750 amino acids, about 480 amino acids to about 700 amino acids, about 480 amino acids to about 650 amino acids, about 480 amino acids to about 600 amino acids, about 480 amino acids to about 550 amino acids, about 480 amino acids to about 500 amino acids, about 500 amino acids to about 3000 amino acids, about 500 amino acids to about 2500 amino acids, about 500 amino acids to about 2000 amino acids, about 500 amino acids to about 1500 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 3000 amino acids, about 550 amino acids to about 2500 amino acids, about 550 amino acids to about 2000 amino acids, about 550 amino acids to about 1500 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 3000 amino acids, about 600 amino acids to about 2500 amino acids, about 600 amino acids to about 2000 amino acids, about 600 amino acids to about 1500 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 3000 amino acids, about 650 amino acids to about 2500 amino acids, about 650 amino acids to about 2000 amino acids, about 650 amino acids to about 1500 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 3000 amino acids, about 700 amino acids to about 2500 amino acids, about 700 amino acids to about 2000 amino acids, about 700 amino acids to about 1500 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 3000 amino acids, about 750 amino acids to about 2500 amino acids, about 750 amino acids to about 2000 amino acids, about 750 amino acids to about 1500 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 3000 amino acids, about 800 amino acids to about 2500 amino acids, about 800 amino acids to about 2000 amino acids, about 800 amino acids to about 1500 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 3000 amino acids, about 850 amino acids to about 2500 amino acids, about 850 amino acids to about 2000 amino acids, about 850 amino acids to about 1500 amino acids, about 850 amino acids to about 1000 amino acids, about 850 amino acids to about 950 amino acids, about 850 amino acids to about 900 amino acids, about 900 amino acids to about 3000 amino acids, about 900 amino acids to about 2500 amino acids, about 900 amino acids to about 2000 amino acids, about 900 amino acids to about 1500 amino acids, about 900 amino acids to about 1000 amino acids, about 900 amino acids to about 950 amino acids, about 950 amino acids to about 3000 amino acids, about 950 amino acids to about 2500 amino acids, about 950 amino acids to about 2000 amino acids, about 950 amino acids to about 1500 amino acids, about 950 amino acids to about 1000 amino acids, about 1000 amino acids to about 3000 amino acids, about 1000 amino acids to about 2500 amino acids, about 1000 amino acids to about 2000 amino acids, about 1000 amino acids to about 1500 amino acids, about 1500 amino acids to about 3000 amino acids, about 1500 amino acids to about 2500 amino acids, about 1500 amino acids to about 2000 amino acids, about 2000 amino acids to about 3000 amino acids, about 2000 amino acids to about 2500 amino acids, or about 2500 amino acids to about 3000 amino acids.

In some embodiments of any of the multi-chain chimeric polypeptides described herein, the first target-binding domain (e.g., any of the first target-binding domains described herein) and the soluble tissue factor domain (e.g., any of the exemplary soluble tissue factor domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the first target-binding domain (e.g., any of the exemplary first target-binding domains described herein) and the soluble tissue factor domain (e.g., any of the exemplary soluble tissue factor domains described herein) in the first chimeric polypeptide.

In some embodiments of any of the multi-chain chimeric polypeptides described herein, the soluble tissue factor domain (e.g., any of the exemplary soluble tissue factor domains described herein) and the first domain of the pair of affinity domains (e.g., any of the exemplary first domains of any of the exemplary pairs of affinity domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the soluble tissue factor domain (e.g., any of the exemplary soluble tissue factor domains described herein) and the first domain of the pair of affinity domains (e.g., any of the exemplary first domains of any of the exemplary pairs of affinity domains described herein) in the first chimeric polypeptide.

In some embodiments of any of the multi-chain chimeric polypeptides described herein, the second domain of the pair of affinity domains (e.g., any of the exemplary second domains of any of the exemplary pairs of affinity domains described herein) and the second target-binding domain (e.g., any of the exemplary second target-binding domains described herein) directly abut each other in the second chimeric polypeptide. In some embodiments of any of the multi-chain chimeric polypeptides described herein, the second chimeric polypeptide further comprises a linker sequence (e.g., any of the exemplary linker sequences described herein or known in the art) between the second domain of the pair of affinity domains (e.g., any of the exemplary second domains of any of the exemplary pairs of affinity domains described herein) and the second target-binding domain (e.g., any of the exemplary second target-binding domains described herein) in the second chimeric polypeptide.

In some embodiments of any of the multi-chain chimeric polypeptides described herein, the first target-binding domain, the second target-binding domain, and/or the one or more additional target-binding domains can each independent have a total number of amino acids of about 5 amino acids to about 1000 amino acids, about 5 amino acids to about 950 amino acids, about 5 amino acids to about 900 amino acids, about 5 amino acids to about 850 amino acids, about 5 amino acids to about 800 amino acids, about 5 amino acids to about 750 amino acids, about 5 amino acids to about 700 amino acids, about 5 amino acids to about 650 amino acids, about 5 amino acids to about 600 amino acids, about 5 amino acids to about 550 amino acids, about 5 amino acids to about 500 amino acids, about 5 amino acids to about 450 amino acids, about 5 amino acids to about 400 amino acids, about 5 amino acids to about 350 amino acids, about 5 amino acids to about 300 amino acids, about 5 amino acids to about 280 amino acids, about 5 amino acids to about 260 amino acids, about 5 amino acids to about 240 amino acids, about 5 amino acids to about 220 amino acids, about 5 amino acids to about 200 amino acids, about 5 amino acids to about 195 amino acids, about 5 amino acids to about 190 amino acids, about 5 amino acids to about 185 amino acids, about 5 amino acids to about 180 amino acids, about 5 amino acids to about 175 amino acids, about 5 amino acids to about 170 amino acids, about 5 amino acids to about 165 amino acids, about 5 amino acids to about 160 amino acids, about 5 amino acids to about 155 amino acids, about 5 amino acids to about 150 amino acids, about 5 amino acids to about 145 amino acids, about 5 amino acids to about 140 amino acids, about 5 amino acids to about 135 amino acids, about 5 amino acids to about 130 amino acids, about 5 amino acids to about 125 amino acids, about 5 amino acids to about 120 amino acids, about 5 amino acids to about 115 amino acids, about 5 amino acids to about 110 amino acids, about 5 amino acids to about 105 amino acids, about 5 amino acids to about 100 amino acids, about 5 amino acids to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 1000 amino acids, about 10 amino acids to about 950 amino acids, about 10 amino acids to about 900 amino acids, about 10 amino acids to about 850 amino acids, about 10 amino acids to about 800 amino acids, about 10 amino acids to about 750 amino acids, about 10 amino acids to about 700 amino acids, about 10 amino acids to about 650 amino acids, about 10 amino acids to about 600 amino acids, about 10 amino acids to about 550 amino acids, about 10 amino acids to about 500 amino acids, about 10 amino acids to about 450 amino acids, about 10 amino acids to about 400 amino acids, about 10 amino acids to about 350 amino acids, about 10 amino acids to about 300 amino acids, about 10 amino acids to about 280 amino acids, about 10 amino acids to about 260 amino acids, about 10 amino acids to about 240 amino acids, about 10 amino acids to about 220 amino acids, about 10 amino acids to about 200 amino acids, about 10 amino acids to about 195 amino acids, about 10 amino acids to about 190 amino acids, about 10 amino acids to about 185 amino acids, about 10 amino acids to about 180 amino acids, about 10 amino acids to about 175 amino acids, about 10 amino acids to about 170 amino acids, about 10 amino acids to about 165 amino acids, about 10 amino acids to about 160 amino acids, about 10 amino acids to about 155 amino acids, about 10 amino acids to about 150 amino acids, about 10 amino acids to about 145 amino acids, about 10 amino acids to about 140 amino acids, about 10 amino acids to about 135 amino acids, about 10 amino acids to about 130 amino acids, about 10 amino acids to about 125 amino acids, about 10 amino acids to about 120 amino acids, about 10 amino acids to about 115 amino acids, about 10 amino acids to about 110 amino acids, about 10 amino acids to about 105 amino acids, about 10 amino acids to about 100 amino acids, about 10 amino acids to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 1000 amino acids, about 15 amino acids to about 950 amino acids, about 15 amino acids to about 900 amino acids, about 15 amino acids to about 850 amino acids, about 15 amino acids to about 800 amino acids, about 15 amino acids to about 750 amino acids, about 15 amino acids to about 700 amino acids, about 15 amino acids to about 650 amino acids, about 15 amino acids to about 600 amino acids, about 15 amino acids to about 550 amino acids, about 15 amino acids to about 500 amino acids, about 15 amino acids to about 450 amino acids, about 15 amino acids to about 400 amino acids, about 15 amino acids to about 350 amino acids, about 15 amino acids to about 300 amino acids, about 15 amino acids to about 280 amino acids, about 15 amino acids to about 260 amino acids, about 15 amino acids to about 240 amino acids, about 15 amino acids to about 220 amino acids, about 15 amino acids to about 200 amino acids, about 15 amino acids to about 195 amino acids, about 15 amino acids to about 190 amino acids, about 15 amino acids to about 185 amino acids, about 15 amino acids to about 180 amino acids, about 15 amino acids to about 175 amino acids, about 15 amino acids to about 170 amino acids, about 15 amino acids to about 165 amino acids, about 15 amino acids to about 160 amino acids, about 15 amino acids to about 155 amino acids, about 15 amino acids to about 150 amino acids, about 15 amino acids to about 145 amino acids, about 15 amino acids to about 140 amino acids, about 15 amino acids to about 135 amino acids, about 15 amino acids to about 130 amino acids, about 15 amino acids to about 125 amino acids, about 15 amino acids to about 120 amino acids, about 15 amino acids to about 115 amino acids, about 15 amino acids to about 110 amino acids, about 15 amino acids to about 105 amino acids, about 15 amino acids to about 100 amino acids, about 15 amino acids to about 95 amino acids, about 15 amino acids to about 90 amino acids, about 15 amino acids to about 85 amino acids, about 15 amino acids to about 80 amino acids, about 15 amino acids to about 75 amino acids, about 15 amino acids to about 70 amino acids, about 15 amino acids to about 65 amino acids, about 15 amino acids to about 60 amino acids, about 15 amino acids to about 55 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 1000 amino acids, about 20 amino acids to about 950 amino acids, about 20 amino acids to about 900 amino acids, about 20 amino acids to about 850 amino acids, about 20 amino acids to about 800 amino acids, about 20 amino acids to about 750 amino acids, about 20 amino acids to about 700 amino acids, about 20 amino acids to about 650 amino acids, about 20 amino acids to about 600 amino acids, about 20 amino acids to about 550 amino acids, about 20 amino acids to about 500 amino acids, about 20 amino acids to about 450 amino acids, about 20 amino acids to about 400 amino acids, about 20 amino acids to about 350 amino acids, about 20 amino acids to about 300 amino acids, about 20 amino acids to about 280 amino acids, about 20 amino acids to about 260 amino acids, about 20 amino acids to about 240 amino acids, about 20 amino acids to about 220 amino acids, about 20 amino acids to about 200 amino acids, about 20 amino acids to about 195 amino acids, about 20 amino acids to about 190 amino acids, about 20 amino acids to about 185 amino acids, about 20 amino acids to about 180 amino acids, about 20 amino acids to about 175 amino acids, about 20 amino acids to about 170 amino acids, about 20 amino acids to about 165 amino acids, about 20 amino acids to about 160 amino acids, about 20 amino acids to about 155 amino acids, about 20 amino acids to about 150 amino acids, about 20 amino acids to about 145 amino acids, about 20 amino acids to about 140 amino acids, about 20 amino acids to about 135 amino acids, about 20 amino acids to about 130 amino acids, about 20 amino acids to about 125 amino acids, about 20 amino acids to about 120 amino acids, about 20 amino acids to about 115 amino acids, about 20 amino acids to about 110 amino acids, about 20 amino acids to about 105 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 1000 amino acids, about 25 amino acids to about 950 amino acids, about 25 amino acids to about 900 amino acids, about 25 amino acids to about 850 amino acids, about 25 amino acids to about 800 amino acids, about 25 amino acids to about 750 amino acids, about 25 amino acids to about 700 amino acids, about 25 amino acids to about 650 amino acids, about 25 amino acids to about 600 amino acids, about 25 amino acids to about 550 amino acids, about 25 amino acids to about 500 amino acids, about 25 amino acids to about 450 amino acids, about 25 amino acids to about 400 amino acids, about 25 amino acids to about 350 amino acids, about 25 amino acids to about 300 amino acids, about 25 amino acids to about 280 amino acids, about 25 amino acids to about 260 amino acids, about 25 amino acids to about 240 amino acids, about 25 amino acids to about 220 amino acids, about 25 amino acids to about 200 amino acids, about 25 amino acids to about 195 amino acids, about 25 amino acids to about 190 amino acids, about 25 amino acids to about 185 amino acids, about 25 amino acids to about 180 amino acids, about 25 amino acids to about 175 amino acids, about 25 amino acids to about 170 amino acids, about 25 amino acids to about 165 amino acids, about 25 amino acids to about 160 amino acids, about 25 amino acids to about 155 amino acids, about 25 amino acids to about 150 amino acids, about 25 amino acids to about 145 amino acids, about 25 amino acids to about 140 amino acids, about 25 amino acids to about 135 amino acids, about 25 amino acids to about 130 amino acids, about 25 amino acids to about 125 amino acids, about 25 amino acids to about 120 amino acids, about 25 amino acids to about 115 amino acids, about 25 amino acids to about 110 amino acids, about 25 amino acids to about 105 amino acids, about 25 amino acids to about 100 amino acids, about 25 amino acids to about 95 amino acids, about 25 amino acids to about 90 amino acids, about 25 amino acids to about 85 amino acids, about 25 amino acids to about 80 amino acids, about 25 amino acids to about 75 amino acids, about 25 amino acids to about 70 amino acids, about 25 amino acids to about 65 amino acids, about 25 amino acids to about 60 amino acids, about 25 amino acids to about 55 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 1000 amino acids, about 30 amino acids to about 950 amino acids, about 30 amino acids to about 900 amino acids, about 30 amino acids to about 850 amino acids, about 30 amino acids to about 800 amino acids, about 30 amino acids to about 750 amino acids, about 30 amino acids to about 700 amino acids, about 30 amino acids to about 650 amino acids, about 30 amino acids to about 600 amino acids, about 30 amino acids to about 550 amino acids, about 30 amino acids to about 500 amino acids, about 30 amino acids to about 450 amino acids, about 30 amino acids to about 400 amino acids, about 30 amino acids to about 350 amino acids, about 30 amino acids to about 300 amino acids, about 30 amino acids to about 280 amino acids, about 30 amino acids to about 260 amino acids, about 30 amino acids to about 240 amino acids, about 30 amino acids to about 220 amino acids, about 30 amino acids to about 200 amino acids, about 30 amino acids to about 195 amino acids, about 30 amino acids to about 190 amino acids, about 30 amino acids to about 185 amino acids, about 30 amino acids to about 180 amino acids, about 30 amino acids to about 175 amino acids, about 30 amino acids to about 170 amino acids, about 30 amino acids to about 165 amino acids, about 30 amino acids to about 160 amino acids, about 30 amino acids to about 155 amino acids, about 30 amino acids to about 150 amino acids, about 30 amino acids to about 145 amino acids, about 30 amino acids to about 140 amino acids, about 30 amino acids to about 135 amino acids, about 30 amino acids to about 130 amino acids, about 30 amino acids to about 125 amino acids, about 30 amino acids to about 120 amino acids, about 30 amino acids to about 115 amino acids, about 30 amino acids to about 110 amino acids, about 30 amino acids to about 105 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 35 amino acids to about 1000 amino acids, about 35 amino acids to about 950 amino acids, about 35 amino acids to about 900 amino acids, about 35 amino acids to about 850 amino acids, about 35 amino acids to about 800 amino acids, about 35 amino acids to about 750 amino acids, about 35 amino acids to about 700 amino acids, about 35 amino acids to about 650 amino acids, about 35 amino acids to about 600 amino acids, about 35 amino acids to about 550 amino acids, about 35 amino acids to about 500 amino acids, about 35 amino acids to about 450 amino acids, about 35 amino acids to about 400 amino acids, about 35 amino acids to about 350 amino acids, about 35 amino acids to about 300 amino acids, about 35 amino acids to about 280 amino acids, about 35 amino acids to about 260 amino acids, about 35 amino acids to about 240 amino acids, about 35 amino acids to about 220 amino acids, about 35 amino acids to about 200 amino acids, about 35 amino acids to about 195 amino acids, about 35 amino acids to about 190 amino acids, about 35 amino acids to about 185 amino acids, about 35 amino acids to about 180 amino acids, about 35 amino acids to about 175 amino acids, about 35 amino acids to about 170 amino acids, about 35 amino acids to about 165 amino acids, about 35 amino acids to about 160 amino acids, about 35 amino acids to about 155 amino acids, about 35 amino acids to about 150 amino acids, about 35 amino acids to about 145 amino acids, about 35 amino acids to about 140 amino acids, about 35 amino acids to about 135 amino acids, about 35 amino acids to about 130 amino acids, about 35 amino acids to about 125 amino acids, about 35 amino acids to about 120 amino acids, about 35 amino acids to about 115 amino acids, about 35 amino acids to about 110 amino acids, about 35 amino acids to about 105 amino acids, about 35 amino acids to about 100 amino acids, about 35 amino acids to about 95 amino acids, about 35 amino acids to about 90 amino acids, about 35 amino acids to about 85 amino acids, about 35 amino acids to about 80 amino acids, about 35 amino acids to about 75 amino acids, about 35 amino acids to about 70 amino acids, about 35 amino acids to about 65 amino acids, about 35 amino acids to about 60 amino acids, about 35 amino acids to about 55 amino acids, about 35 amino acids to about 50 amino acids, about 35 amino acids to about 45 amino acids, about 35 amino acids to about 40 amino acids, about 40 amino acids to about 1000 amino acids, about 40 amino acids to about 950 amino acids, about 40 amino acids to about 900 amino acids, about 40 amino acids to about 850 amino acids, about 40 amino acids to about 800 amino acids, about 40 amino acids to about 750 amino acids, about 40 amino acids to about 700 amino acids, about 40 amino acids to about 650 amino acids, about 40 amino acids to about 600 amino acids, about 40 amino acids to about 550 amino acids, about 40 amino acids to about 500 amino acids, about 40 amino acids to about 450 amino acids, about 40 amino acids to about 400 amino acids, about 40 amino acids to about 350 amino acids, about 40 amino acids to about 300 amino acids, about 40 amino acids to about 280 amino acids, about 40 amino acids to about 260 amino acids, about 40 amino acids to about 240 amino acids, about 40 amino acids to about 220 amino acids, about 40 amino acids to about 200 amino acids, about 40 amino acids to about 195 amino acids, about 40 amino acids to about 190 amino acids, about 40 amino acids to about 185 amino acids, about 40 amino acids to about 180 amino acids, about 40 amino acids to about 175 amino acids, about 40 amino acids to about 170 amino acids, about 40 amino acids to about 165 amino acids, about 40 amino acids to about 160 amino acids, about 40 amino acids to about 155 amino acids, about 40 amino acids to about 150 amino acids, about 40 amino acids to about 145 amino acids, about 40 amino acids to about 140 amino acids, about 40 amino acids to about 135 amino acids, about 40 amino acids to about 130 amino acids, about 40 amino acids to about 125 amino acids, about 40 amino acids to about 120 amino acids, about 40 amino acids to about 115 amino acids, about 40 amino acids to about 110 amino acids, about 40 amino acids to about 105 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 45 amino acids to about 1000 amino acids, about 45 amino acids to about 950 amino acids, about 45 amino acids to about 900 amino acids, about 45 amino acids to about 850 amino acids, about 45 amino acids to about 800 amino acids, about 45 amino acids to about 750 amino acids, about 45 amino acids to about 700 amino acids, about 45 amino acids to about 650 amino acids, about 45 amino acids to about 600 amino acids, about 45 amino acids to about 550 amino acids, about 45 amino acids to about 500 amino acids, about 45 amino acids to about 450 amino acids, about 45 amino acids to about 400 amino acids, about 45 amino acids to about 350 amino acids, about 45 amino acids to about 300 amino acids, about 45 amino acids to about 280 amino acids, about 45 amino acids to about 260 amino acids, about 45 amino acids to about 240 amino acids, about 45 amino acids to about 220 amino acids, about 45 amino acids to about 200 amino acids, about 45 amino acids to about 195 amino acids, about 45 amino acids to about 190 amino acids, about 45 amino acids to about 185 amino acids, about 45 amino acids to about 180 amino acids, about 45 amino acids to about 175 amino acids, about 45 amino acids to about 170 amino acids, about 45 amino acids to about 165 amino acids, about 45 amino acids to about 160 amino acids, about 45 amino acids to about 155 amino acids, about 45 amino acids to about 150 amino acids, about 45 amino acids to about 145 amino acids, about 45 amino acids to about 140 amino acids, about 45 amino acids to about 135 amino acids, about 45 amino acids to about 130 amino acids, about 45 amino acids to about 125 amino acids, about 45 amino acids to about 120 amino acids, about 45 amino acids to about 115 amino acids, about 45 amino acids to about 110 amino acids, about 45 amino acids to about 105 amino acids, about 45 amino acids to about 100 amino acids, about 45 amino acids to about 95 amino acids, about 45 amino acids to about 90 amino acids, about 45 amino acids to about 85 amino acids, about 45 amino acids to about 80 amino acids, about 45 amino acids to about 75 amino acids, about 45 amino acids to about 70 amino acids, about 45 amino acids to about 65 amino acids, about 45 amino acids to about 60 amino acids, about 45 amino acids to about 55 amino acids, about 45 amino acids to about 50 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 280 amino acids, about 50 amino acids to about 260 amino acids, about 50 amino acids to about 240 amino acids, about 50 amino acids to about 220 amino acids, about 50 amino acids to about 200 amino acids, about 50 amino acids to about 195 amino acids, about 50 amino acids to about 190 amino acids, about 50 amino acids to about 185 amino acids, about 50 amino acids to about 180 amino acids, about 50 amino acids to about 175 amino acids, about 50 amino acids to about 170 amino acids, about 50 amino acids to about 165 amino acids, about 50 amino acids to about 160 amino acids, about 50 amino acids to about 155 amino acids, about 50 amino acids to about 150 amino acids, about 50 amino acids to about 145 amino acids, about 50 amino acids to about 140 amino acids, about 50 amino acids to about 135 amino acids, about 50 amino acids to about 130 amino acids, about 50 amino acids to about 125 amino acids, about 50 amino acids to about 120 amino acids, about 50 amino acids to about 115 amino acids, about 50 amino acids to about 110 amino acids, about 50 amino acids to about 105 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 55 amino acids to about 1000 amino acids, about 55 amino acids to about 950 amino acids, about 55 amino acids to about 900 amino acids, about 55 amino acids to about 850 amino acids, about 55 amino acids to about 800 amino acids, about 55 amino acids to about 750 amino acids, about 55 amino acids to about 700 amino acids, about 55 amino acids to about 650 amino acids, about 55 amino acids to about 600 amino acids, about 55 amino acids to about 550 amino acids, about 55 amino acids to about 500 amino acids, about 55 amino acids to about 450 amino acids, about 55 amino acids to about 400 amino acids, about 55 amino acids to about 350 amino acids, about 55 amino acids to about 300 amino acids, about 55 amino acids to about 280 amino acids, about 55 amino acids to about 260 amino acids, about 55 amino acids to about 240 amino acids, about 55 amino acids to about 220 amino acids, about 55 amino acids to about 200 amino acids, about 55 amino acids to about 195 amino acids, about 55 amino acids to about 190 amino acids, about 55 amino acids to about 185 amino acids, about 55 amino acids to about 180 amino acids, about 55 amino acids to about 175 amino acids, about 55 amino acids to about 170 amino acids, about 55 amino acids to about 165 amino acids, about 55 amino acids to about 160 amino acids, about 55 amino acids to about 155 amino acids, about 55 amino acids to about 150 amino acids, about 55 amino acids to about 145 amino acids, about 55 amino acids to about 140 amino acids, about 55 amino acids to about 135 amino acids, about 55 amino acids to about 130 amino acids, about 55 amino acids to about 125 amino acids, about 55 amino acids to about 120 amino acids, about 55 amino acids to about 115 amino acids, about 55 amino acids to about 110 amino acids, about 55 amino acids to about 105 amino acids, about 55 amino acids to about 100 amino acids, about 55 amino acids to about 95 amino acids, about 55 amino acids to about 90 amino acids, about 55 amino acids to about 85 amino acids, about 55 amino acids to about 80 amino acids, about 55 amino acids to about 75 amino acids, about 55 amino acids to about 70 amino acids, about 55 amino acids to about 65 amino acids, about 55 amino acids to about 60 amino acids, about 60 amino acids to about 1000 amino acids, about 60 amino acids to about 950 amino acids, about 60 amino acids to about 900 amino acids, about 60 amino acids to about 850 amino acids, about 60 amino acids to about 800 amino acids, about 60 amino acids to about 750 amino acids, about 60 amino acids to about 700 amino acids, about 60 amino acids to about 650 amino acids, about 60 amino acids to about 600 amino acids, about 60 amino acids to about 550 amino acids, about 60 amino acids to about 500 amino acids, about 60 amino acids to about 450 amino acids, about 60 amino acids to about 400 amino acids, about 60 amino acids to about 350 amino acids, about 60 amino acids to about 300 amino acids, about 60 amino acids to about 280 amino acids, about 60 amino acids to about 260 amino acids, about 60 amino acids to about 240 amino acids, about 60 amino acids to about 220 amino acids, about 60 amino acids to about 200 amino acids, about 60 amino acids to about 195 amino acids, about 60 amino acids to about 190 amino acids, about 60 amino acids to about 185 amino acids, about 60 amino acids to about 180 amino acids, about 60 amino acids to about 175 amino acids, about 60 amino acids to about 170 amino acids, about 60 amino acids to about 165 amino acids, about 60 amino acids to about 160 amino acids, about 60 amino acids to about 155 amino acids, about 60 amino acids to about 150 amino acids, about 60 amino acids to about 145 amino acids, about 60 amino acids to about 140 amino acids, about 60 amino acids to about 135 amino acids, about 60 amino acids to about 130 amino acids, about 60 amino acids to about 125 amino acids, about 60 amino acids to about 120 amino acids, about 60 amino acids to about 115 amino acids, about 60 amino acids to about 110 amino acids, about 60 amino acids to about 105 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 65 amino acids to about 1000 amino acids, about 65 amino acids to about 950 amino acids, about 65 amino acids to about 900 amino acids, about 65 amino acids to about 850 amino acids, about 65 amino acids to about 800 amino acids, about 65 amino acids to about 750 amino acids, about 65 amino acids to about 700 amino acids, about 65 amino acids to about 650 amino acids, about 65 amino acids to about 600 amino acids, about 65 amino acids to about 550 amino acids, about 65 amino acids to about 500 amino acids, about 65 amino acids to about 450 amino acids, about 65 amino acids to about 400 amino acids, about 65 amino acids to about 350 amino acids, about 65 amino acids to about 300 amino acids, about 65 amino acids to about 280 amino acids, about 65 amino acids to about 260 amino acids, about 65 amino acids to about 240 amino acids, about 65 amino acids to about 220 amino acids, about 65 amino acids to about 200 amino acids, about 65 amino acids to about 195 amino acids, about 65 amino acids to about 190 amino acids, about 65 amino acids to about 185 amino acids, about 65 amino acids to about 180 amino acids, about 65 amino acids to about 175 amino acids, about 65 amino acids to about 170 amino acids, about 65 amino acids to about 165 amino acids, about 65 amino acids to about 160 amino acids, about 65 amino acids to about 155 amino acids, about 65 amino acids to about 150 amino acids, about 65 amino acids to about 145 amino acids, about 65 amino acids to about 140 amino acids, about 65 amino acids to about 135 amino acids, about 65 amino acids to about 130 amino acids, about 65 amino acids to about 125 amino acids, about 65 amino acids to about 120 amino acids, about 65 amino acids to about 115 amino acids, about 65 amino acids to about 110 amino acids, about 65 amino acids to about 105 amino acids, about 65 amino acids to about 100 amino acids, about 65 amino acids to about 95 amino acids, about 65 amino acids to about 90 amino acids, about 65 amino acids to about 85 amino acids, about 65 amino acids to about 80 amino acids, about 65 amino acids to about 75 amino acids, about 65 amino acids to about 70 amino acids, about 70 amino acids to about 1000 amino acids, about 70 amino acids to about 950 amino acids, about 70 amino acids to about 900 amino acids, about 70 amino acids to about 850 amino acids, about 70 amino acids to about 800 amino acids, about 70 amino acids to about 750 amino acids, about 70 amino acids to about 700 amino acids, about 70 amino acids to about 650 amino acids, about 70 amino acids to about 600 amino acids, about 70 amino acids to about 550 amino acids, about 70 amino acids to about 500 amino acids, about 70 amino acids to about 450 amino acids, about 70 amino acids to about 400 amino acids, about 70 amino acids to about 350 amino acids, about 70 amino acids to about 300 amino acids, about 70 amino acids to about 280 amino acids, about 70 amino acids to about 260 amino acids, about 70 amino acids to about 240 amino acids, about 70 amino acids to about 220 amino acids, about 70 amino acids to about 200 amino acids, about 70 amino acids to about 195 amino acids, about 70 amino acids to about 190 amino acids, about 70 amino acids to about 185 amino acids, about 70 amino acids to about 180 amino acids, about 70 amino acids to about 175 amino acids, about 70 amino acids to about 170 amino acids, about 70 amino acids to about 165 amino acids, about 70 amino acids to about 160 amino acids, about 70 amino acids to about 155 amino acids, about 70 amino acids to about 150 amino acids, about 70 amino acids to about 145 amino acids, about 70 amino acids to about 140 amino acids, about 70 amino acids to about 135 amino acids, about 70 amino acids to about 130 amino acids, about 70 amino acids to about 125 amino acids, about 70 amino acids to about 120 amino acids, about 70 amino acids to about 115 amino acids, about 70 amino acids to about 110 amino acids, about 70 amino acids to about 105 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 70 amino acids to about 75 amino acids, about 75 amino acids to about 1000 amino acids, about 75 amino acids to about 950 amino acids, about 75 amino acids to about 900 amino acids, about 75 amino acids to about 850 amino acids, about 75 amino acids to about 800 amino acids, about 75 amino acids to about 750 amino acids, about 75 amino acids to about 700 amino acids, about 75 amino acids to about 650 amino acids, about 75 amino acids to about 600 amino acids, about 75 amino acids to about 550 amino acids, about 75 amino acids to about 500 amino acids, about 75 amino acids to about 450 amino acids, about 75 amino acids to about 400 amino acids, about 75 amino acids to about 350 amino acids, about 75 amino acids to about 300 amino acids, about 75 amino acids to about 280 amino acids, about 75 amino acids to about 260 amino acids, about 75 amino acids to about 240 amino acids, about 75 amino acids to about 220 amino acids, about 75 amino acids to about 200 amino acids, about 75 amino acids to about 195 amino acids, about 75 amino acids to about 190 amino acids, about 75 amino acids to about 185 amino acids, about 75 amino acids to about 180 amino acids, about 75 amino acids to about 175 amino acids, about 75 amino acids to about 170 amino acids, about 75 amino acids to about 165 amino acids, about 75 amino acids to about 160 amino acids, about 75 amino acids to about 155 amino acids, about 75 amino acids to about 150 amino acids, about 75 amino acids to about 145 amino acids, about 75 amino acids to about 140 amino acids, about 75 amino acids to about 135 amino acids, about 75 amino acids to about 130 amino acids, about 75 amino acids to about 125 amino acids, about 75 amino acids to about 120 amino acids, about 75 amino acids to about 115 amino acids, about 75 amino acids to about 110 amino acids, about 75 amino acids to about 105 amino acids, about 75 amino acids to about 100 amino acids, about 75 amino acids to about 95 amino acids, about 75 amino acids to about 90 amino acids, about 75 amino acids to about 85 amino acids, about 75 amino acids to about 80 amino acids, about 80 amino acids to about 1000 amino acids, about 80 amino acids to about 950 amino acids, about 80 amino acids to about 900 amino acids, about 80 amino acids to about 850 amino acids, about 80 amino acids to about 800 amino acids, about 80 amino acids to about 750 amino acids, about 80 amino acids to about 700 amino acids, about 80 amino acids to about 650 amino acids, about 80 amino acids to about 600 amino acids, about 80 amino acids to about 550 amino acids, about 80 amino acids to about 500 amino acids, about 80 amino acids to about 450 amino acids, about 80 amino acids to about 400 amino acids, about 80 amino acids to about 350 amino acids, about 80 amino acids to about 300 amino acids, about 80 amino acids to about 280 amino acids, about 80 amino acids to about 260 amino acids, about 80 amino acids to about 240 amino acids, about 80 amino acids to about 220 amino acids, about 80 amino acids to about 200 amino acids, about 80 amino acids to about 195 amino acids, about 80 amino acids to about 190 amino acids, about 80 amino acids to about 185 amino acids, about 80 amino acids to about 180 amino acids, about 80 amino acids to about 175 amino acids, about 80 amino acids to about 170 amino acids, about 80 amino acids to about 165 amino acids, about 80 amino acids to about 160 amino acids, about 80 amino acids to about 155 amino acids, about 80 amino acids to about 150 amino acids, about 80 amino acids to about 145 amino acids, about 80 amino acids to about 140 amino acids, about 80 amino acids to about 135 amino acids, about 80 amino acids to about 130 amino acids, about 80 amino acids to about 125 amino acids, about 80 amino acids to about 120 amino acids, about 80 amino acids to about 115 amino acids, about 80 amino acids to about 110 amino acids, about 80 amino acids to about 105 amino acids, about 80 amino acids to about 100 amino acids, about 80 amino acids to about 95 amino acids, about 80 amino acids to about 90 amino acids, about 80 amino acids to about 85 amino acids, about 85 amino acids to about 1000 amino acids, about 85 amino acids to about 950 amino acids, about 85 amino acids to about 900 amino acids, about 85 amino acids to about 850 amino acids, about 85 amino acids to about 800 amino acids, about 85 amino acids to about 750 amino acids, about 85 amino acids to about 700 amino acids, about 85 amino acids to about 650 amino acids, about 85 amino acids to about 600 amino acids, about 85 amino acids to about 550 amino acids, about 85 amino acids to about 500 amino acids, about 85 amino acids to about 450 amino acids, about 85 amino acids to about 400 amino acids, about 85 amino acids to about 350 amino acids, about 85 amino acids to about 300 amino acids, about 85 amino acids to about 280 amino acids, about 85 amino acids to about 260 amino acids, about 85 amino acids to about 240 amino acids, about 85 amino acids to about 220 amino acids, about 85 amino acids to about 200 amino acids, about 85 amino acids to about 195 amino acids, about 85 amino acids to about 190 amino acids, about 85 amino acids to about 185 amino acids, about 85 amino acids to about 180 amino acids, about 85 amino acids to about 175 amino acids, about 85 amino acids to about 170 amino acids, about 85 amino acids to about 165 amino acids, about 85 amino acids to about 160 amino acids, about 85 amino acids to about 155 amino acids, about 85 amino acids to about 150 amino acids, about 85 amino acids to about 145 amino acids, about 85 amino acids to about 140 amino acids, about 85 amino acids to about 135 amino acids, about 85 amino acids to about 130 amino acids, about 85 amino acids to about 125 amino acids, about 85 amino acids to about 120 amino acids, about 85 amino acids to about 115 amino acids, about 85 amino acids to about 110 amino acids, about 85 amino acids to about 105 amino acids, about 85 amino acids to about 100 amino acids, about 85 amino acids to about 95 amino acids, about 85 amino acids to about 90 amino acids, about 90 amino acids to about 1000 amino acids, about 90 amino acids to about 950 amino acids, about 90 amino acids to about 900 amino acids, about 90 amino acids to about 850 amino acids, about 90 amino acids to about 800 amino acids, about 90 amino acids to about 750 amino acids, about 90 amino acids to about 700 amino acids, about 90 amino acids to about 650 amino acids, about 90 amino acids to about 600 amino acids, about 90 amino acids to about 550 amino acids, about 90 amino acids to about 500 amino acids, about 90 amino acids to about 450 amino acids, about 90 amino acids to about 400 amino acids, about 90 amino acids to about 350 amino acids, about 90 amino acids to about 300 amino acids, about 90 amino acids to about 280 amino acids, about 90 amino acids to about 260 amino acids, about 90 amino acids to about 240 amino acids, about 90 amino acids to about 220 amino acids, about 90 amino acids to about 200 amino acids, about 90 amino acids to about 195 amino acids, about 90 amino acids to about 190 amino acids, about 90 amino acids to about 185 amino acids, about 90 amino acids to about 180 amino acids, about 90 amino acids to about 175 amino acids, about 90 amino acids to about 170 amino acids, about 90 amino acids to about 165 amino acids, about 90 amino acids to about 160 amino acids, about 90 amino acids to about 155 amino acids, about 90 amino acids to about 150 amino acids, about 90 amino acids to about 145 amino acids, about 90 amino acids to about 140 amino acids, about 90 amino acids to about 135 amino acids, about 90 amino acids to about 130 amino acids, about 90 amino acids to about 125 amino acids, about 90 amino acids to about 120 amino acids, about 90 amino acids to about 115 amino acids, about 90 amino acids to about 110 amino acids, about 90 amino acids to about 105 amino acids, about 90 amino acids to about 100 amino acids, about 90 amino acids to about 95 amino acids, about 95 amino acids to about 1000 amino acids, about 95 amino acids to about 950 amino acids, about 95 amino acids to about 900 amino acids, about 95 amino acids to about 850 amino acids, about 95 amino acids to about 800 amino acids, about 95 amino acids to about 750 amino acids, about 95 amino acids to about 700 amino acids, about 95 amino acids to about 650 amino acids, about 95 amino acids to about 600 amino acids, about 95 amino acids to about 550 amino acids, about 95 amino acids to about 500 amino acids, about 95 amino acids to about 450 amino acids, about 95 amino acids to about 400 amino acids, about 95 amino acids to about 350 amino acids, about 95 amino acids to about 300 amino acids, about 95 amino acids to about 280 amino acids, about 95 amino acids to about 260 amino acids, about 95 amino acids to about 240 amino acids, about 95 amino acids to about 220 amino acids, about 95 amino acids to about 200 amino acids, about 95 amino acids to about 195 amino acids, about 95 amino acids to about 190 amino acids, about 95 amino acids to about 185 amino acids, about 95 amino acids to about 180 amino acids, about 95 amino acids to about 175 amino acids, about 95 amino acids to about 170 amino acids, about 95 amino acids to about 165 amino acids, about 95 amino acids to about 160 amino acids, about 95 amino acids to about 155 amino acids, about 95 amino acids to about 150 amino acids, about 95 amino acids to about 145 amino acids, about 95 amino acids to about 140 amino acids, about 95 amino acids to about 135 amino acids, about 95 amino acids to about 130 amino acids, about 95 amino acids to about 125 amino acids, about 95 amino acids to about 120 amino acids, about 95 amino acids to about 115 amino acids, about 95 amino acids to about 110 amino acids, about 95 amino acids to about 105 amino acids, about 95 amino acids to about 100 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 280 amino acids, about 100 amino acids to about 260 amino acids, about 100 amino acids to about 240 amino acids, about 100 amino acids to about 220 amino acids, about 100 amino acids to about 200 amino acids, about 100 amino acids to about 195 amino acids, about 100 amino acids to about 190 amino acids, about 100 amino acids to about 185 amino acids, about 100 amino acids to about 180 amino acids, about 100 amino acids to about 175 amino acids, about 100 amino acids to about 170 amino acids, about 100 amino acids to about 165 amino acids, about 100 amino acids to about 160 amino acids, about 100 amino acids to about 155 amino acids, about 100 amino acids to about 150 amino acids, about 100 amino acids to about 145 amino acids, about 100 amino acids to about 140 amino acids, about 100 amino acids to about 135 amino acids, about 100 amino acids to about 130 amino acids, about 100 amino acids to about 125 amino acids, about 100 amino acids to about 120 amino acids, about 100 amino acids to about 115 amino acids, about 100 amino acids to about 110 amino acids, about 100 amino acids to about 105 amino acids, about 105 amino acids to about 1000 amino acids, about 105 amino acids to about 950 amino acids, about 105 amino acids to about 900 amino acids, about 105 amino acids to about 850 amino acids, about 105 amino acids to about 800 amino acids, about 105 amino acids to about 750 amino acids, about 105 amino acids to about 700 amino acids, about 105 amino acids to about 650 amino acids, about 105 amino acids to about 600 amino acids, about 105 amino acids to about 550 amino acids, about 105 amino acids to about 500 amino acids, about 105 amino acids to about 450 amino acids, about 105 amino acids to about 400 amino acids, about 105 amino acids to about 350 amino acids, about 105 amino acids to about 300 amino acids, about 105 amino acids to about 280 amino acids, about 105 amino acids to about 260 amino acids, about 105 amino acids to about 240 amino acids, about 105 amino acids to about 220 amino acids, about 105 amino acids to about 200 amino acids, about 105 amino acids to about 195 amino acids, about 105 amino acids to about 190 amino acids, about 105 amino acids to about 185 amino acids, about 105 amino acids to about 180 amino acids, about 105 amino acids to about 175 amino acids, about 105 amino acids to about 170 amino acids, about 105 amino acids to about 165 amino acids, about 105 amino acids to about 160 amino acids, about 105 amino acids to about 155 amino acids, about 105 amino acids to about 150 amino acids, about 105 amino acids to about 145 amino acids, about 105 amino acids to about 140 amino acids, about 105 amino acids to about 135 amino acids, about 105 amino acids to about 130 amino acids, about 105 amino acids to about 125 amino acids, about 105 amino acids to about 120 amino acids, about 105 amino acids to about 115 amino acids, about 105 amino acids to about 110 amino acids, about 110 amino acids to about 1000 amino acids, about 110 amino acids to about 950 amino acids, about 110 amino acids to about 900 amino acids, about 110 amino acids to about 850 amino acids, about 110 amino acids to about 800 amino acids, about 110 amino acids to about 750 amino acids, about 110 amino acids to about 700 amino acids, about 110 amino acids to about 650 amino acids, about 110 amino acids to about 600 amino acids, about 110 amino acids to about 550 amino acids, about 110 amino acids to about 500 amino acids, about 110 amino acids to about 450 amino acids, about 110 amino acids to about 400 amino acids, about 110 amino acids to about 350 amino acids, about 110 amino acids to about 300 amino acids, about 110 amino acids to about 280 amino acids, about 110 amino acids to about 260 amino acids, about 110 amino acids to about 240 amino acids, about 110 amino acids to about 220 amino acids, about 110 amino acids to about 200 amino acids, about 110 amino acids to about 195 amino acids, about 110 amino acids to about 190 amino acids, about 110 amino acids to about 185 amino acids, about 110 amino acids to about 180 amino acids, about 110 amino acids to about 175 amino acids, about 110 amino acids to about 170 amino acids, about 110 amino acids to about 165 amino acids, about 110 amino acids to about 160 amino acids, about 110 amino acids to about 155 amino acids, about 110 amino acids to about 150 amino acids, about 110 amino acids to about 145 amino acids, about 110 amino acids to about 140 amino acids, about 110 amino acids to about 135 amino acids, about 110 amino acids to about 130 amino acids, about 110 amino acids to about 125 amino acids, about 110 amino acids to about 120 amino acids, about 110 amino acids to about 115 amino acids, about 115 amino acids to about 1000 amino acids, about 115 amino acids to about 950 amino acids, about 115 amino acids to about 900 amino acids, about 115 amino acids to about 850 amino acids, about 115 amino acids to about 800 amino acids, about 115 amino acids to about 750 amino acids, about 115 amino acids to about 700 amino acids, about 115 amino acids to about 650 amino acids, about 115 amino acids to about 600 amino acids, about 115 amino acids to about 550 amino acids, about 115 amino acids to about 500 amino acids, about 115 amino acids to about 450 amino acids, about 115 amino acids to about 400 amino acids, about 115 amino acids to about 350 amino acids, about 115 amino acids to about 300 amino acids, about 115 amino acids to about 280 amino acids, about 115 amino acids to about 260 amino acids, about 115 amino acids to about 240 amino acids, about 115 amino acids to about 220 amino acids, about 115 amino acids to about 200 amino acids, about 115 amino acids to about 195 amino acids, about 115 amino acids to about 190 amino acids, about 115 amino acids to about 185 amino acids, about 115 amino acids to about 180 amino acids, about 115 amino acids to about 175 amino acids, about 115 amino acids to about 170 amino acids, about 115 amino acids to about 165 amino acids, about 115 amino acids to about 160 amino acids, about 115 amino acids to about 155 amino acids, about 115 amino acids to about 150 amino acids, about 115 amino acids to about 145 amino acids, about 115 amino acids to about 140 amino acids, about 115 amino acids to about 135 amino acids, about 115 amino acids to about 130 amino acids, about 115 amino acids to about 125 amino acids, about 115 amino acids to about 120 amino acids, about 120 amino acids to about 1000 amino acids, about 120 amino acids to about 950 amino acids, about 120 amino acids to about 900 amino acids, about 120 amino acids to about 850 amino acids, about 120 amino acids to about 800 amino acids, about 120 amino acids to about 750 amino acids, about 120 amino acids to about 700 amino acids, about 120 amino acids to about 650 amino acids, about 120 amino acids to about 600 amino acids, about 120 amino acids to about 550 amino acids, about 120 amino acids to about 500 amino acids, about 120 amino acids to about 450 amino acids, about 120 amino acids to about 400 amino acids, about 120 amino acids to about 350 amino acids, about 120 amino acids to about 300 amino acids, about 120 amino acids to about 280 amino acids, about 120 amino acids to about 260 amino acids, about 120 amino acids to about 240 amino acids, about 120 amino acids to about 220 amino acids, about 120 amino acids to about 200 amino acids, about 120 amino acids to about 195 amino acids, about 120 amino acids to about 190 amino acids, about 120 amino acids to about 185 amino acids, about 120 amino acids to about 180 amino acids, about 120 amino acids to about 175 amino acids, about 120 amino acids to about 170 amino acids, about 120 amino acids to about 165 amino acids, about 120 amino acids to about 160 amino acids, about 120 amino acids to about 155 amino acids, about 120 amino acids to about 150 amino acids, about 120 amino acids to about 145 amino acids, about 120 amino acids to about 140 amino acids, about 120 amino acids to about 135 amino acids, about 120 amino acids to about 130 amino acids, about 120 amino acids to about 125 amino acids, about 125 amino acids to about 1000 amino acids, about 125 amino acids to about 950 amino acids, about 125 amino acids to about 900 amino acids, about 125 amino acids to about 850 amino acids, about 125 amino acids to about 800 amino acids, about 125 amino acids to about 750 amino acids, about 125 amino acids to about 700 amino acids, about 125 amino acids to about 650 amino acids, about 125 amino acids to about 600 amino acids, about 125 amino acids to about 550 amino acids, about 125 amino acids to about 500 amino acids, about 125 amino acids to about 450 amino acids, about 125 amino acids to about 400 amino acids, about 125 amino acids to about 350 amino acids, about 125 amino acids to about 300 amino acids, about 125 amino acids to about 280 amino acids, about 125 amino acids to about 260 amino acids, about 125 amino acids to about 240 amino acids, about 125 amino acids to about 220 amino acids, about 125 amino acids to about 200 amino acids, about 125 amino acids to about 195 amino acids, about 125 amino acids to about 190 amino acids, about 125 amino acids to about 185 amino acids, about 125 amino acids to about 180 amino acids, about 125 amino acids to about 175 amino acids, about 125 amino acids to about 170 amino acids, about 125 amino acids to about 165 amino acids, about 125 amino acids to about 160 amino acids, about 125 amino acids to about 155 amino acids, about 125 amino acids to about 150 amino acids, about 125 amino acids to about 145 amino acids, about 125 amino acids to about 140 amino acids, about 125 amino acids to about 135 amino acids, about 125 amino acids to about 130 amino acids, about 130 amino acids to about 1000 amino acids, about 130 amino acids to about 950 amino acids, about 130 amino acids to about 900 amino acids, about 130 amino acids to about 850 amino acids, about 130 amino acids to about 800 amino acids, about 130 amino acids to about 750 amino acids, about 130 amino acids to about 700 amino acids, about 130 amino acids to about 650 amino acids, about 130 amino acids to about 600 amino acids, about 130 amino acids to about 550 amino acids, about 130 amino acids to about 500 amino acids, about 130 amino acids to about 450 amino acids, about 130 amino acids to about 400 amino acids, about 130 amino acids to about 350 amino acids, about 130 amino acids to about 300 amino acids, about 130 amino acids to about 280 amino acids, about 130 amino acids to about 260 amino acids, about 130 amino acids to about 240 amino acids, about 130 amino acids to about 220 amino acids, about 130 amino acids to about 200 amino acids, about 130 amino acids to about 195 amino acids, about 130 amino acids to about 190 amino acids, about 130 amino acids to about 185 amino acids, about 130 amino acids to about 180 amino acids, about 130 amino acids to about 175 amino acids, about 130 amino acids to about 170 amino acids, about 130 amino acids to about 165 amino acids, about 130 amino acids to about 160 amino acids, about 130 amino acids to about 155 amino acids, about 130 amino acids to about 150 amino acids, about 130 amino acids to about 145 amino acids, about 130 amino acids to about 140 amino acids, about 130 amino acids to about 135 amino acids, about 135 amino acids to about 1000 amino acids, about 135 amino acids to about 950 amino acids, about 135 amino acids to about 900 amino acids, about 135 amino acids to about 850 amino acids, about 135 amino acids to about 800 amino acids, about 135 amino acids to about 750 amino acids, about 135 amino acids to about 700 amino acids, about 135 amino acids to about 650 amino acids, about 135 amino acids to about 600 amino acids, about 135 amino acids to about 550 amino acids, about 135 amino acids to about 500 amino acids, about 135 amino acids to about 450 amino acids, about 135 amino acids to about 400 amino acids, about 135 amino acids to about 350 amino acids, about 135 amino acids to about 300 amino acids, about 135 amino acids to about 280 amino acids, about 135 amino acids to about 260 amino acids, about 135 amino acids to about 240 amino acids, about 135 amino acids to about 220 amino acids, about 135 amino acids to about 200 amino acids, about 135 amino acids to about 195 amino acids, about 135 amino acids to about 190 amino acids, about 135 amino acids to about 185 amino acids, about 135 amino acids to about 180 amino acids, about 135 amino acids to about 175 amino acids, about 135 amino acids to about 170 amino acids, about 135 amino acids to about 165 amino acids, about 135 amino acids to about 160 amino acids, about 135 amino acids to about 155 amino acids, about 135 amino acids to about 150 amino acids, about 135 amino acids to about 145 amino acids, about 135 amino acids to about 140 amino acids, about 140 amino acids to about 1000 amino acids, about 140 amino acids to about 950 amino acids, about 140 amino acids to about 900 amino acids, about 140 amino acids to about 850 amino acids, about 140 amino acids to about 800 amino acids, about 140 amino acids to about 750 amino acids, about 140 amino acids to about 700 amino acids, about 140 amino acids to about 650 amino acids, about 140 amino acids to about 600 amino acids, about 140 amino acids to about 550 amino acids, about 140 amino acids to about 500 amino acids, about 140 amino acids to about 450 amino acids, about 140 amino acids to about 400 amino acids, about 140 amino acids to about 350 amino acids, about 140 amino acids to about 300 amino acids, about 140 amino acids to about 280 amino acids, about 140 amino acids to about 260 amino acids, about 140 amino acids to about 240 amino acids, about 140 amino acids to about 220 amino acids, about 140 amino acids to about 200 amino acids, about 140 amino acids to about 195 amino acids, about 140 amino acids to about 190 amino acids, about 140 amino acids to about 185 amino acids, about 140 amino acids to about 180 amino acids, about 140 amino acids to about 175 amino acids, about 140 amino acids to about 170 amino acids, about 140 amino acids to about 165 amino acids, about 140 amino acids to about 160 amino acids, about 140 amino acids to about 155 amino acids, about 140 amino acids to about 150 amino acids, about 140 amino acids to about 145 amino acids, about 145 amino acids to about 1000 amino acids, about 145 amino acids to about 950 amino acids, about 145 amino acids to about 900 amino acids, about 145 amino acids to about 850 amino acids, about 145 amino acids to about 800 amino acids, about 145 amino acids to about 750 amino acids, about 145 amino acids to about 700 amino acids, about 145 amino acids to about 650 amino acids, about 145 amino acids to about 600 amino acids, about 145 amino acids to about 550 amino acids, about 145 amino acids to about 500 amino acids, about 145 amino acids to about 450 amino acids, about 145 amino acids to about 400 amino acids, about 145 amino acids to about 350 amino acids, about 145 amino acids to about 300 amino acids, about 145 amino acids to about 280 amino acids, about 145 amino acids to about 260 amino acids, about 145 amino acids to about 240 amino acids, about 145 amino acids to about 220 amino acids, about 145 amino acids to about 200 amino acids, about 145 amino acids to about 195 amino acids, about 145 amino acids to about 190 amino acids, about 145 amino acids to about 185 amino acids, about 145 amino acids to about 180 amino acids, about 145 amino acids to about 175 amino acids, about 145 amino acids to about 170 amino acids, about 145 amino acids to about 165 amino acids, about 145 amino acids to about 160 amino acids, about 145 amino acids to about 155 amino acids, about 145 amino acids to about 150 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 280 amino acids, about 150 amino acids to about 260 amino acids, about 150 amino acids to about 240 amino acids, about 150 amino acids to about 220 amino acids, about 150 amino acids to about 200 amino acids, about 150 amino acids to about 195 amino acids, about 150 amino acids to about 190 amino acids, about 150 amino acids to about 185 amino acids, about 150 amino acids to about 180 amino acids, about 150 amino acids to about 175 amino acids, about 150 amino acids to about 170 amino acids, about 150 amino acids to about 165 amino acids, about 150 amino acids to about 160 amino acids, about 150 amino acids to about 155 amino acids, about 155 amino acids to about 1000 amino acids, about 155 amino acids to about 950 amino acids, about 155 amino acids to about 900 amino acids, about 155 amino acids to about 850 amino acids, about 155 amino acids to about 800 amino acids, about 155 amino acids to about 750 amino acids, about 155 amino acids to about 700 amino acids, about 155 amino acids to about 650 amino acids, about 155 amino acids to about 600 amino acids, about 155 amino acids to about 550 amino acids, about 155 amino acids to about 500 amino acids, about 155 amino acids to about 450 amino acids, about 155 amino acids to about 400 amino acids, about 155 amino acids to about 350 amino acids, about 155 amino acids to about 300 amino acids, about 155 amino acids to about 280 amino acids, about 155 amino acids to about 260 amino acids, about 155 amino acids to about 240 amino acids, about 155 amino acids to about 220 amino acids, about 155 amino acids to about 200 amino acids, about 155 amino acids to about 195 amino acids, about 155 amino acids to about 190 amino acids, about 155 amino acids to about 185 amino acids, about 155 amino acids to about 180 amino acids, about 155 amino acids to about 175 amino acids, about 155 amino acids to about 170 amino acids, about 155 amino acids to about 165 amino acids, about 155 amino acids to about 160 amino acids, about 160 amino acids to about 1000 amino acids, about 160 amino acids to about 950 amino acids, about 160 amino acids to about 900 amino acids, about 160 amino acids to about 850 amino acids, about 160 amino acids to about 800 amino acids, about 160 amino acids to about 750 amino acids, about 160 amino acids to about 700 amino acids, about 160 amino acids to about 650 amino acids, about 160 amino acids to about 600 amino acids, about 160 amino acids to about 550 amino acids, about 160 amino acids to about 500 amino acids, about 160 amino acids to about 450 amino acids, about 160 amino acids to about 400 amino acids, about 160 amino acids to about 350 amino acids, about 160 amino acids to about 300 amino acids, about 160 amino acids to about 280 amino acids, about 160 amino acids to about 260 amino acids, about 160 amino acids to about 240 amino acids, about 160 amino acids to about 220 amino acids, about 160 amino acids to about 200 amino acids, about 160 amino acids to about 195 amino acids, about 160 amino acids to about 190 amino acids, about 160 amino acids to about 185 amino acids, about 160 amino acids to about 180 amino acids, about 160 amino acids to about 175 amino acids, about 160 amino acids to about 170 amino acids, about 160 amino acids to about 165 amino acids, about 165 amino acids to about 1000 amino acids, about 165 amino acids to about 950 amino acids, about 165 amino acids to about 900 amino acids, about 165 amino acids to about 850 amino acids, about 165 amino acids to about 800 amino acids, about 165 amino acids to about 750 amino acids, about 165 amino acids to about 700 amino acids, about 165 amino acids to about 650 amino acids, about 165 amino acids to about 600 amino acids, about 165 amino acids to about 550 amino acids, about 165 amino acids to about 500 amino acids, about 165 amino acids to about 450 amino acids, about 165 amino acids to about 400 amino acids, about 165 amino acids to about 350 amino acids, about 165 amino acids to about 300 amino acids, about 165 amino acids to about 280 amino acids, about 165 amino acids to about 260 amino acids, about 165 amino acids to about 240 amino acids, about 165 amino acids to about 220 amino acids, about 165 amino acids to about 200 amino acids, about 165 amino acids to about 195 amino acids, about 165 amino acids to about 190 amino acids, about 165 amino acids to about 185 amino acids, about 165 amino acids to about 180 amino acids, about 165 amino acids to about 175 amino acids, about 165 amino acids to about 170 amino acids, about 170 amino acids to about 1000 amino acids, about 170 amino acids to about 950 amino acids, about 170 amino acids to about 900 amino acids, about 170 amino acids to about 850 amino acids, about 170 amino acids to about 800 amino acids, about 170 amino acids to about 750 amino acids, about 170 amino acids to about 700 amino acids, about 170 amino acids to about 650 amino acids, about 170 amino acids to about 600 amino acids, about 170 amino acids to about 550 amino acids, about 170 amino acids to about 500 amino acids, about 170 amino acids to about 450 amino acids, about 170 amino acids to about 400 amino acids, about 170 amino acids to about 350 amino acids, about 170 amino acids to about 300 amino acids, about 170 amino acids to about 280 amino acids, about 170 amino acids to about 260 amino acids, about 170 amino acids to about 240 amino acids, about 170 amino acids to about 220 amino acids, about 170 amino acids to about 200 amino acids, about 170 amino acids to about 195 amino acids, about 170 amino acids to about 190 amino acids, about 170 amino acids to about 185 amino acids, about 170 amino acids to about 180 amino acids, about 170 amino acids to about 175 amino acids, about 175 amino acids to about 1000 amino acids, about 175 amino acids to about 950 amino acids, about 175 amino acids to about 900 amino acids, about 175 amino acids to about 850 amino acids, about 175 amino acids to about 800 amino acids, about 175 amino acids to about 750 amino acids, about 175 amino acids to about 700 amino acids, about 175 amino acids to about 650 amino acids, about 175 amino acids to about 600 amino acids, about 175 amino acids to about 550 amino acids, about 175 amino acids to about 500 amino acids, about 175 amino acids to about 450 amino acids, about 175 amino acids to about 400 amino acids, about 175 amino acids to about 350 amino acids, about 175 amino acids to about 300 amino acids, about 175 amino acids to about 280 amino acids, about 175 amino acids to about 260 amino acids, about 175 amino acids to about 240 amino acids, about 175 amino acids to about 220 amino acids, about 175 amino acids to about 200 amino acids, about 175 amino acids to about 195 amino acids, about 175 amino acids to about 190 amino acids, about 175 amino acids to about 185 amino acids, about 175 amino acids to about 180 amino acids, about 180 amino acids to about 1000 amino acids, about 180 amino acids to about 950 amino acids, about 180 amino acids to about 900 amino acids, about 180 amino acids to about 850 amino acids, about 180 amino acids to about 800 amino acids, about 180 amino acids to about 750 amino acids, about 180 amino acids to about 700 amino acids, about 180 amino acids to about 650 amino acids, about 180 amino acids to about 600 amino acids, about 180 amino acids to about 550 amino acids, about 180 amino acids to about 500 amino acids, about 180 amino acids to about 450 amino acids, about 180 amino acids to about 400 amino acids, about 180 amino acids to about 350 amino acids, about 180 amino acids to about 300 amino acids, about 180 amino acids to about 280 amino acids, about 180 amino acids to about 260 amino acids, about 180 amino acids to about 240 amino acids, about 180 amino acids to about 220 amino acids, about 180 amino acids to about 200 amino acids, about 180 amino acids to about 195 amino acids, about 180 amino acids to about 190 amino acids, about 180 amino acids to about 185 amino acids, about 185 amino acids to about 1000 amino acids, about 185 amino acids to about 950 amino acids, about 185 amino acids to about 900 amino acids, about 185 amino acids to about 850 amino acids, about 185 amino acids to about 800 amino acids, about 185 amino acids to about 750 amino acids, about 185 amino acids to about 700 amino acids, about 185 amino acids to about 650 amino acids, about 185 amino acids to about 600 amino acids, about 185 amino acids to about 550 amino acids, about 185 amino acids to about 500 amino acids, about 185 amino acids to about 450 amino acids, about 185 amino acids to about 400 amino acids, about 185 amino acids to about 350 amino acids, about 185 amino acids to about 300 amino acids, about 185 amino acids to about 280 amino acids, about 185 amino acids to about 260 amino acids, about 185 amino acids to about 240 amino acids, about 185 amino acids to about 220 amino acids, about 185 amino acids to about 200 amino acids, about 185 amino acids to about 195 amino acids, about 185 amino acids to about 190 amino acids, about 190 amino acids to about 1000 amino acids, about 190 amino acids to about 950 amino acids, about 190 amino acids to about 900 amino acids, about 190 amino acids to about 850 amino acids, about 190 amino acids to about 800 amino acids, about 190 amino acids to about 750 amino acids, about 190 amino acids to about 700 amino acids, about 190 amino acids to about 650 amino acids, about 190 amino acids to about 600 amino acids, about 190 amino acids to about 550 amino acids, about 190 amino acids to about 500 amino acids, about 190 amino acids to about 450 amino acids, about 190 amino acids to about 400 amino acids, about 190 amino acids to about 350 amino acids, about 190 amino acids to about 300 amino acids, about 190 amino acids to about 280 amino acids, about 190 amino acids to about 260 amino acids, about 190 amino acids to about 240 amino acids, about 190 amino acids to about 220 amino acids, about 190 amino acids to about 200 amino acids, about 190 amino acids to about 195 amino acids, about 195 amino acids to about 1000 amino acids, about 195 amino acids to about 950 amino acids, about 195 amino acids to about 900 amino acids, about 195 amino acids to about 850 amino acids, about 195 amino acids to about 800 amino acids, about 195 amino acids to about 750 amino acids, about 195 amino acids to about 700 amino acids, about 195 amino acids to about 650 amino acids, about 195 amino acids to about 600 amino acids, about 195 amino acids to about 550 amino acids, about 195 amino acids to about 500 amino acids, about 195 amino acids to about 450 amino acids, about 195 amino acids to about 400 amino acids, about 195 amino acids to about 350 amino acids, about 195 amino acids to about 300 amino acids, about 195 amino acids to about 280 amino acids, about 195 amino acids to about 260 amino acids, about 195 amino acids to about 240 amino acids, about 195 amino acids to about 220 amino acids, about 195 amino acids to about 200 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 280 amino acids, about 200 amino acids to about 260 amino acids, about 200 amino acids to about 240 amino acids, about 200 amino acids to about 220 amino acids, about 220 amino acids to about 1000 amino acids, about 220 amino acids to about 950 amino acids, about 220 amino acids to about 900 amino acids, about 220 amino acids to about 850 amino acids, about 220 amino acids to about 800 amino acids, about 220 amino acids to about 750 amino acids, about 220 amino acids to about 700 amino acids, about 220 amino acids to about 650 amino acids, about 220 amino acids to about 600 amino acids, about 220 amino acids to about 550 amino acids, about 220 amino acids to about 500 amino acids, about 220 amino acids to about 450 amino acids, about 220 amino acids to about 400 amino acids, about 220 amino acids to about 350 amino acids, about 220 amino acids to about 300 amino acids, about 220 amino acids to about 280 amino acids, about 220 amino acids to about 260 amino acids, about 220 amino acids to about 240 amino acids, about 240 amino acids to about 1000 amino acids, about 240 amino acids to about 950 amino acids, about 240 amino acids to about 900 amino acids, about 240 amino acids to about 850 amino acids, about 240 amino acids to about 800 amino acids, about 240 amino acids to about 750 amino acids, about 240 amino acids to about 700 amino acids, about 240 amino acids to about 650 amino acids, about 240 amino acids to about 600 amino acids, about 240 amino acids to about 550 amino acids, about 240 amino acids to about 500 amino acids, about 240 amino acids to about 450 amino acids, about 240 amino acids to about 400 amino acids, about 240 amino acids to about 350 amino acids, about 240 amino acids to about 300 amino acids, about 240 amino acids to about 280 amino acids, about 240 amino acids to about 260 amino acids, about 260 amino acids to about 1000 amino acids, about 260 amino acids to about 950 amino acids, about 260 amino acids to about 900 amino acids, about 260 amino acids to about 850 amino acids, about 260 amino acids to about 800 amino acids, about 260 amino acids to about 750 amino acids, about 260 amino acids to about 700 amino acids, about 260 amino acids to about 650 amino acids, about 260 amino acids to about 600 amino acids, about 260 amino acids to about 550 amino acids, about 260 amino acids to about 500 amino acids, about 260 amino acids to about 450 amino acids, about 260 amino acids to about 400 amino acids, about 260 amino acids to about 350 amino acids, about 260 amino acids to about 300 amino acids, about 260 amino acids to about 280 amino acids, about 280 amino acids to about 1000 amino acids, about 280 amino acids to about 950 amino acids, about 280 amino acids to about 900 amino acids, about 280 amino acids to about 850 amino acids, about 280 amino acids to about 800 amino acids, about 280 amino acids to about 750 amino acids, about 280 amino acids to about 700 amino acids, about 280 amino acids to about 650 amino acids, about 280 amino acids to about 600 amino acids, about 280 amino acids to about 550 amino acids, about 280 amino acids to about 500 amino acids, about 280 amino acids to about 450 amino acids, about 280 amino acids to about 400 amino acids, about 280 amino acids to about 350 amino acids, about 280 amino acids to about 300 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 1000 amino acids, about 350 amino acids to about 950 amino acids, about 350 amino acids to about 900 amino acids, about 350 amino acids to about 850 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 1000 amino acids, about 450 amino acids to about 950 amino acids, about 450 amino acids to about 900 amino acids, about 450 amino acids to about 850 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 1000 amino acids, about 850 amino acids to about 950 amino acids, about 850 amino acids to about 900 amino acids, about 900 amino acids to about 1000 amino acids, about 900 amino acids to about 950 amino acids, or about 950 amino acids to about 1000 amino acids.

Any of the target-binding domains described herein can bind to a ligand of TGF-βRII with a dissociation equilibrium constant (K_{D}) of less than 1×10⁻⁷ M, less than 1×10⁻⁸ M, less than 1×10⁻⁹ M, less than 1×10⁻¹⁰ M, less than 1×10⁻¹¹ M, less than 1×10⁻¹²M, or less than 1×10⁻¹³ M.

In some embodiments, the antigen-binding protein construct provided herein can bind to an identifying antigen with a K_{D} of about 1×10⁻³ M to about 1×10⁻⁵ M, about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷M to about 1×10⁻⁹ M, about 1×10⁻⁸ M to about 1×10⁻¹⁰ M, or about 1×10⁻⁹ M to about 1×10⁻¹¹ M (inclusive).

Any of the target-binding domains described herein can bind to a ligand of TGF-βRII (e.g., TGF-β) with a K_{D} of between about 1 pM to about 30 nM (e.g., about 1 pM to about 25 nM, about 1 pM to about 20 nM, about 1 pM to about 15 nM, about 1 pM to about 10 nM, about 1 pM to about 5 nM, about 1 pM to about 2 nM, about 1 pM to about 1 nM, about 1 pM to about 950 pM, about 1 pM to about 900 pM, about 1 pM to about 850 pM, about 1 pM to about 800 pM, about 1 pM to about 750 pM, about 1 pM to about 700 pM, about 1 pM to about 650 pM, about 1 pM to about 600 pM, about 1 pM to about 550 pM, about 1 pM to about 500 pM, about 1 pM to about 450 pM, about 1 pM to about 400 pM, about 1 pM to about 350 pM, about 1 pM to about 300 pM, about 1 pM to about 250 pM, about 1 pM to about 200 pM, about 1 pM to about 150 pM, about 1 pM to about 100 pM, about 1 pM to about 90 pM, about 1 pM to about 80 pM, about 1 pM to about 70 pM, about 1 pM to about 60 pM, about 1 pM to about 50 pM, about 1 pM to about 40 pM, about 1 pM to about 30 pM, about 1 pM to about 20 pM, about 1 pM to about 10 pM, about 1 pM to about 5 pM, about 1 pM to about 4 pM, about 1 pM to about 3 pM, about 1 pM to about 2 pM, about 2 pM to about 30 nM, about 2 pM to about 25 nM, about 2 pM to about 20 nM, about 2 pM to about 15 nM, about 2 pM to about 10 nM, about 2 pM to about 5 nM, about 2 pM to about 2 nM, about 2 pM to about 1 nM, about 2 pM to about 950 pM, about 2 pM to about 900 pM, about 2 pM to about 850 pM, about 2 pM to about 800 pM, about 2 pM to about 750 pM, about 2 pM to about 700 pM, about 2 pM to about 650 pM, about 2 pM to about 600 pM, about 2 pM to about 550 pM, about 2 pM to about 500 pM, about 2 pM to about 450 pM, about 2 pM to about 400 pM, about 2 pM to about 350 pM, about 2 pM to about 300 pM, about 2 pM to about 250 pM, about 2 pM to about 200 pM, about 2 pM to about 150 pM, about 2 pM to about 100 pM, about 2 pM to about 90 pM, about 2 pM to about 80 pM, about 2 pM to about 70 pM, about 2 pM to about 60 pM, about 2 pM to about 50 pM, about 2 pM to about 40 pM, about 2 pM to about 30 pM, about 2 pM to about 20 pM, about 2 pM to about 10 pM, about 2 pM to about 5 pM, about 2 pM to about 4 pM, about 2 pM to about 3 pM, about 5 pM to about 30 nM, about 5 pM to about 25 nM, about 5 pM to about 20 nM, about 5 pM to about 15 nM, about 5 pM to about 10 nM, about 5 pM to about 5 nM, about 5 pM to about 2 nM, about 5 pM to about 1 nM, about 5 pM to about 950 pM, about 5 pM to about 900 pM, about 5 pM to about 850 pM, about 5 pM to about 800 pM, about 5 pM to about 750 pM, about 5 pM to about 700 pM, about 5 pM to about 650 pM, about 5 pM to about 600 pM, about 5 pM to about 550 pM, about 5 pM to about 500 pM, about 5 pM to about 450 pM, about 5 pM to about 400 pM, about 5 pM to about 350 pM, about 5 pM to about 300 pM, about 5 pM to about 250 pM, about 5 pM to about 200 pM, about 5 pM to about 150 pM, about 5 pM to about 100 pM, about 5 pM to about 90 pM, about 5 pM to about 80 pM, about 5 pM to about 70 pM, about 5 pM to about 60 pM, about 5 pM to about 50 pM, about 5 pM to about 40 pM, about 5 pM to about 30 pM, about 5 pM to about 20 pM, about 5 pM to about 10 pM, about 10 pM to about 30 nM, about 10 pM to about 25 nM, about 10 pM to about 20 nM, about 10 pM to about 15 nM, about 10 pM to about 10 nM, about 10 pM to about 5 nM, about 10 pM to about 2 nM, about 10 pM to about 1 nM, about 10 pM to about 950 pM, about 10 pM to about 900 pM, about 10 pM to about 850 pM, about 10 pM to about 800 pM, about 10 pM to about 750 pM, about 10 pM to about 700 pM, about 10 pM to about 650 pM, about 10 pM to about 600 pM, about 10 pM to about 550 pM, about 10 pM to about 500 pM, about 10 pM to about 450 pM, about 10 pM to about 400 pM, about 10 pM to about 350 pM, about 10 pM to about 300 pM, about 10 pM to about 250 pM, about 10 pM to about 200 pM, about 10 pM to about 150 pM, about 10 pM to about 100 pM, about 10 pM to about 90 pM, about 10 pM to about 80 pM, about 10 pM to about 70 pM, about 10 pM to about 60 pM, about 10 pM to about 50 pM, about 10 pM to about 40 pM, about 10 pM to about 30 pM, about 10 pM to about 20 pM, about 15 pM to about 30 nM, about 15 pM to about 25 nM, about 15 pM to about 20 nM, about 15 pM to about 15 nM, about 15 pM to about 10 nM, about 15 pM to about 5 nM, about 15 pM to about 2 nM, about 15 pM to about 1 nM, about 15 pM to about 950 pM, about 15 pM to about 900 pM, about 15 pM to about 850 pM, about 15 pM to about 800 pM, about 15 pM to about 750 pM, about 15 pM to about 700 pM, about 15 pM to about 650 pM, about 15 pM to about 600 pM, about 15 pM to about 550 pM, about 15 pM to about 500 pM, about 15 pM to about 450 pM, about 15 pM to about 400 pM, about 15 pM to about 350 pM, about 15 pM to about 300 pM, about 15 pM to about 250 pM, about 15 pM to about 200 pM, about 15 pM to about 150 pM, about 15 pM to about 100 pM, about 15 pM to about 90 pM, about 15 pM to about 80 pM, about 15 pM to about 70 pM, about 15 pM to about 60 pM, about 15 pM to about 50 pM, about 15 pM to about 40 pM, about 15 pM to about 30 pM, about 15 pM to about 20 pM, about 20 pM to about 30 nM, about 20 pM to about 25 nM, about 20 pM to about 20 nM, about 20 pM to about 15 nM, about 20 pM to about 10 nM, about 20 pM to about 5 nM, about 20 pM to about 2 nM, about 20 pM to about 1 nM, about 20 pM to about 950 pM, about 20 pM to about 900 pM, about 20 pM to about 850 pM, about 20 pM to about 800 pM, about 20 pM to about 750 pM, about 20 pM to about 700 pM, about 20 pM to about 650 pM, about 20 pM to about 600 pM, about 20 pM to about 550 pM, about 20 pM to about 500 pM, about 20 pM to about 450 pM, about 20 pM to about 400 pM, about 20 pM to about 350 pM, about 20 pM to about 300 pM, about 20 pM to about 250 pM, about 20 pM to about 20 pM, about 200 pM to about 150 pM, about 20 pM to about 100 pM, about 20 pM to about 90 pM, about 20 pM to about 80 pM, about 20 pM to about 70 pM, about 20 pM to about 60 pM, about 20 pM to about 50 pM, about 20 pM to about 40 pM, about 20 pM to about 30 pM, about 30 pM to about 30 nM, about 30 pM to about 25 nM, about 30 pM to about 30 nM, about 30 pM to about 15 nM, about 30 pM to about 10 nM, about 30 pM to about 5 nM, about 30 pM to about 2 nM, about 30 pM to about 1 nM, about 30 pM to about 950 pM, about 30 pM to about 900 pM, about 30 pM to about 850 pM, about 30 pM to about 800 pM, about 30 pM to about 750 pM, about 30 pM to about 700 pM, about 30 pM to about 650 pM, about 30 pM to about 600 pM, about 30 pM to about 550 pM, about 30 pM to about 500 pM, about 30 pM to about 450 pM, about 30 pM to about 400 pM, about 30 pM to about 350 pM, about 30 pM to about 300 pM, about 30 pM to about 250 pM, about 30 pM to about 200 pM, about 30 pM to about 150 pM, about 30 pM to about 100 pM, about 30 pM to about 90 pM, about 30 pM to about 80 pM, about 30 pM to about 70 pM, about 30 pM to about 60 pM, about 30 pM to about 50 pM, about 30 pM to about 40 pM, about 40 pM to about 30 nM, about 40 pM to about 25 nM, about 40 pM to about 30 nM, about 40 pM to about 15 nM, about 40 pM to about 10 nM, about 40 pM to about 5 nM, about 40 pM to about 2 nM, about 40 pM to about 1 nM, about 40 pM to about 950 pM, about 40 pM to about 900 pM, about 40 pM to about 850 pM, about 40 pM to about 800 pM, about 40 pM to about 750 pM, about 40 pM to about 700 pM, about 40 pM to about 650 pM, about 40 pM to about 600 pM, about 40 pM to about 550 pM, about 40 pM to about 500 pM, about 40 pM to about 450 pM, about 40 pM to about 400 pM, about 40 pM to about 350 pM, about 40 pM to about 300 pM, about 40 pM to about 250 pM, about 40 pM to about 200 pM, about 40 pM to about 150 pM, about 40 pM to about 100 pM, about 40 pM to about 90 pM, about 40 pM to about 80 pM, about 40 pM to about 70 pM, about 40 pM to about 60 pM, about 40 pM to about 50 pM, about 50 pM to about 30 nM, about 50 pM to about 25 nM, about 50 pM to about 30 nM, about 50 pM to about 15 nM, about 50 pM to about 10 nM, about 50 pM to about 5 nM, about 50 pM to about 2 nM, about 50 pM to about 1 nM, about 50 pM to about 950 pM, about 50 pM to about 900 pM, about 50 pM to about 850 pM, about 50 pM to about 800 pM, about 50 pM to about 750 pM, about 50 pM to about 700 pM, about 50 pM to about 650 pM, about 50 pM to about 600 pM, about 50 pM to about 550 pM, about 50 pM to about 500 pM, about 50 pM to about 450 pM, about 50 pM to about 400 pM, about 50 pM to about 350 pM, about 50 pM to about 300 pM, about 50 pM to about 250 pM, about 50 pM to about 200 pM, about 50 pM to about 150 pM, about 50 pM to about 100 pM, about 50 pM to about 90 pM, about 50 pM to about 80 pM, about 50 pM to about 70 pM, about 50 pM to about 60 pM, about 60 pM to about 30 nM, about 60 pM to about 25 nM, about 60 pM to about 30 nM, about 60 pM to about 15 nM, about 60 pM to about 10 nM, about 60 pM to about 5 nM, about 60 pM to about 2 nM, about 60 pM to about 1 nM, about 60 pM to about 950 pM, about 60 pM to about 900 pM, about 60 pM to about 850 pM, about 60 pM to about 800 pM, about 60 pM to about 750 pM, about 60 pM to about 700 pM, about 60 pM to about 650 pM, about 60 pM to about 600 pM, about 60 pM to about 550 pM, about 60 pM to about 500 pM, about 60 pM to about 450 pM, about 60 pM to about 400 pM, about 60 pM to about 350 pM, about 60 pM to about 300 pM, about 60 pM to about 250 pM, about 60 pM to about 200 pM, about 60 pM to about 150 pM, about 60 pM to about 100 pM, about 60 pM to about 90 pM, about 60 pM to about 80 pM, about 60 pM to about 70 pM, about 70 pM to about 30 nM, about 70 pM to about 25 nM, about 70 pM to about 30 nM, about 70 pM to about 15 nM, about 70 pM to about 10 nM, about 70 pM to about 5 nM, about 70 pM to about 2 nM, about 70 pM to about 1 nM, about 70 pM to about 950 pM, about 70 pM to about 900 pM, about 70 pM to about 850 pM, about 70 pM to about 800 pM, about 70 pM to about 750 pM, about 70 pM to about 700 pM, about 70 pM to about 650 pM, about 70 pM to about 600 pM, about 70 pM to about 550 pM, about 70 pM to about 500 pM, about 70 pM to about 450 pM, about 70 pM to about 400 pM, about 70 pM to about 350 pM, about 70 pM to about 300 pM, about 70 pM to about 250 pM, about 70 pM to about 200 pM, about 70 pM to about 150 pM, about 70 pM to about 100 pM, about 70 pM to about 90 pM, about 70 pM to about 80 pM, about 80 pM to about 30 nM, about 80 pM to about 25 nM, about 80 pM to about 30 nM, about 80 pM to about 15 nM, about 80 pM to about 10 nM, about 80 pM to about 5 nM, about 80 pM to about 2 nM, about 80 pM to about 1 nM, about 80 pM to about 950 pM, about 80 pM to about 900 pM, about 80 pM to about 850 pM, about 80 pM to about 800 pM, about 80 pM to about 750 pM, about 80 pM to about 700 pM, about 80 pM to about 650 pM, about 80 pM to about 600 pM, about 80 pM to about 550 pM, about 80 pM to about 500 pM, about 80 pM to about 450 pM, about 80 pM to about 400 pM, about 80 pM to about 350 pM, about 80 pM to about 300 pM, about 80 pM to about 250 pM, about 80 pM to about 200 pM, about 80 pM to about 150 pM, about 80 pM to about 100 pM, about 80 pM to about 90 pM, about 90 pM to about 30 nM, about 90 pM to about 25 nM, about 90 pM to about 30 nM, about 90 pM to about 15 nM, about 90 pM to about 10 nM, about 90 pM to about 5 nM, about 90 pM to about 2 nM, about 90 pM to about 1 nM, about 90 pM to about 950 pM, about 90 pM to about 900 pM, about 90 pM to about 850 pM, about 90 pM to about 800 pM, about 90 pM to about 750 pM, about 90 pM to about 700 pM, about 90 pM to about 650 pM, about 90 pM to about 600 pM, about 90 pM to about 550 pM, about 90 pM to about 500 pM, about 90 pM to about 450 pM, about 90 pM to about 400 pM, about 90 pM to about 350 pM, about 90 pM to about 300 pM, about 90 pM to about 250 pM, about 90 pM to about 200 pM, about 90 pM to about 150 pM, about 90 pM to about 100 pM, about 100 pM to about 30 nM, about 100 pM to about 25 nM, about 100 pM to about 30 nM, about 100 pM to about 15 nM, about 100 pM to about 10 nM, about 100 pM to about 5 nM, about 100 pM to about 2 nM, about 100 pM to about 1 nM, about 100 pM to about 950 pM, about 100 pM to about 900 pM, about 100 pM to about 850 pM, about 100 pM to about 800 pM, about 100 pM to about 750 pM, about 100 pM to about 700 pM, about 100 pM to about 650 pM, about 100 pM to about 600 pM, about 100 pM to about 550 pM, about 100 pM to about 500 pM, about 100 pM to about 450 pM, about 100 pM to about 400 pM, about 100 pM to about 350 pM, about 100 pM to about 300 pM, about 100 pM to about 250 pM, about 100 pM to about 200 pM, about 100 pM to about 150 pM, about 150 pM to about 30 nM, about 150 pM to about 25 nM, about 150 pM to about 30 nM, about 150 pM to about 15 nM, about 150 pM to about 10 nM, about 150 pM to about 5 nM, about 150 pM to about 2 nM, about 150 pM to about 1 nM, about 150 pM to about 950 pM, about 150 pM to about 900 pM, about 150 pM to about 850 pM, about 150 pM to about 800 pM, about 150 pM to about 750 pM, about 150 pM to about 700 pM, about 150 pM to about 650 pM, about 150 pM to about 600 pM, about 150 pM to about 550 pM, about 150 pM to about 500 pM, about 150 pM to about 450 pM, about 150 pM to about 400 pM, about 150 pM to about 350 pM, about 150 pM to about 300 pM, about 150 pM to about 250 pM, about 150 pM to about 200 pM, about 200 pM to about 30 nM, about 200 pM to about 25 nM, about 200 pM to about 30 nM, about 200 pM to about 15 nM, about 200 pM to about 10 nM, about 200 pM to about 5 nM, about 200 pM to about 2 nM, about 200 pM to about 1 nM, about 200 pM to about 950 pM, about 200 pM to about 900 pM, about 200 pM to about 850 pM, about 200 pM to about 800 pM, about 200 pM to about 750 pM, about 200 pM to about 700 pM, about 200 pM to about 650 pM, about 200 pM to about 600 pM, about 200 pM to about 550 pM, about 200 pM to about 500 pM, about 200 pM to about 450 pM, about 200 pM to about 400 pM, about 200 pM to about 350 pM, about 200 pM to about 300 pM, about 200 pM to about 250 pM, about 300 pM to about 30 nM, about 300 pM to about 25 nM, about 300 pM to about 30 nM, about 300 pM to about 15 nM, about 300 pM to about 10 nM, about 300 pM to about 5 nM, about 300 pM to about 2 nM, about 300 pM to about 1 nM, about 300 pM to about 950 pM, about 300 pM to about 900 pM, about 300 pM to about 850 pM, about 300 pM to about 800 pM, about 300 pM to about 750 pM, about 300 pM to about 700 pM, about 300 pM to about 650 pM, about 300 pM to about 600 pM, about 300 pM to about 550 pM, about 300 pM to about 500 pM, about 300 pM to about 450 pM, about 300 pM to about 400 pM, about 300 pM to about 350 pM, about 400 pM to about 30 nM, about 400 pM to about 25 nM, about 400 pM to about 30 nM, about 400 pM to about 15 nM, about 400 pM to about 10 nM, about 400 pM to about 5 nM, about 400 pM to about 2 nM, about 400 pM to about 1 nM, about 400 pM to about 950 pM, about 400 pM to about 900 pM, about 400 pM to about 850 pM, about 400 pM to about 800 pM, about 400 pM to about 750 pM, about 400 pM to about 700 pM, about 400 pM to about 650 pM, about 400 pM to about 600 pM, about 400 pM to about 550 pM, about 400 pM to about 500 pM, about 500 pM to about 30 nM, about 500 pM to about 25 nM, about 500 pM to about 30 nM, about 500 pM to about 15 nM, about 500 pM to about 10 nM, about 500 pM to about 5 nM, about 500 pM to about 2 nM, about 500 pM to about 1 nM, about 500 pM to about 950 pM, about 500 pM to about 900 pM, about 500 pM to about 850 pM, about 500 pM to about 800 pM, about 500 pM to about 750 pM, about 500 pM to about 700 pM, about 500 pM to about 650 pM, about 500 pM to about 600 pM, about 500 pM to about 550 pM, about 600 pM to about 30 nM, about 600 pM to about 25 nM, about 600 pM to about 30 nM, about 600 pM to about 15 nM, about 600 pM to about 10 nM, about 600 pM to about 5 nM, about 600 pM to about 2 nM, about 600 pM to about 1 nM, about 600 pM to about 950 pM, about 600 pM to about 900 pM, about 600 pM to about 850 pM, about 600 pM to about 800 pM, about 600 pM to about 750 pM, about 600 pM to about 700 pM, about 600 pM to about 650 pM, about 700 pM to about 30 nM, about 700 pM to about 25 nM, about 700 pM to about 30 nM, about 700 pM to about 15 nM, about 700 pM to about 10 nM, about 700 pM to about 5 nM, about 700 pM to about 2 nM, about 700 pM to about 1 nM, about 700 pM to about 950 pM, about 700 pM to about 900 pM, about 700 pM to about 850 pM, about 700 pM to about 800 pM, about 700 pM to about 750 pM, about 800 pM to about 30 nM, about 800 pM to about 25 nM, about 800 pM to about 30 nM, about 800 pM to about 15 nM, about 800 pM to about 10 nM, about 800 pM to about 5 nM, about 800 pM to about 2 nM, about 800 pM to about 1 nM, about 800 pM to about 950 pM, about 800 pM to about 900 pM, about 800 pM to about 850 pM, about 900 pM to about 30 nM, about 900 pM to about 25 nM, about 900 pM to about 30 nM, about 900 pM to about 15 nM, about 900 pM to about 10 nM, about 900 pM to about 5 nM, about 900 pM to about 2 nM, about 900 pM to about 1 nM, about 900 pM to about 950 pM, about 1 nM to about 30 nM, about 1 nM to about 25 nM, about 1 nM to about 20 nM, about 1 nM to about 15 nM, about 1 nM to about 10 nM, about 1 nM to about 5 nM, about 2 nM to about 30 nM, about 2 nM to about 25 nM, about 2 nM to about 20 nM, about 2 nM to about 15 nM, about 2 nM to about 10 nM, about 2 nM to about 5 nM, about 4 nM to about 30 nM, about 4 nM to about 25 nM, about 4 nM to about 20 nM, about 4 nM to about 15 nM, about 4 nM to about 10 nM, about 4 nM to about 5 nM, about 5 nM to about 30 nM, about 5 nM to about 25 nM, about 5 nM to about 20 nM, about 5 nM to about 15 nM, about 5 nM to about 10 nM, about 10 nM to about 30 nM, about 10 nM to about 25 nM, about 10 nM to about 20 nM, about 10 nM to about 15 nM, about 15 nM to about 30 nM, about 15 nM to about 25 nM, about 15 nM to about 20 nM, about 20 nM to about 30 nM, and about 20 nM to about 25 nM). Any of the target-binding domains described herein can bind to a ligand of TGFβRII with a K_{D} of between about 1 nM to about 10 nM (e.g., about 1 nM to about 9 nM, about 1 nM to about 8 nM, about 1 nM to about 7 nM, about 1 nM to about 6 nM, about 1 nM to about 5 nM, about 1 nM to about 4 nM, about 1 nM to about 3 nM, about 1 nM to about 2 nM, about 2 nM to about 10 nM, about 2 nM to about 9 nM, about 2 nM to about 8 nM, about 2 nM to about 7 nM, about 2 nM to about 6 nM, about 2 nM to about 5 nM, about 2 nM to about 4 nM, about 2 nM to about 3 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 3 nM to about 4 nM, about 4 nM to about 10 nM, about 4 nM to about 9 nM, about 4 nM to about 8 nM, about 4 nM to about 7 nM, about 4 nM to about 6 nM, about 4 nM to about 5 nM, about 5 nM to about 10 nM, about 5 nM to about 9 nM, about 5 nM to about 8 nM, about 5 nM to about 7 nM, about 5 nM to about 6 nM, about 6 nM to about 10 nM, about 6 nM to about 9 nM, about 6 nM to about 8 nM, about 6 nM to about 7 nM, about 7 nM to about 10 nM, about 7 nM to about 9 nM, about 7 nM to about 8 nM, about 8 nM to about 10 nM, about 8 nM to about 9 nM, and about 9 nM to about 10 nM).

A variety of different methods known in the art can be used to determine the K_{D} values of any of the antigen-binding protein constructs described herein (e.g., an electrophoretic mobility shift assay, a filter binding assay, surface plasmon resonance, and a biomolecular binding kinetics assay, etc.).

### Tissue Factor

Human tissue factor is a 263 amino-acid transmembrane protein containing three domains: (1) a 219-amino acid N-terminal extracellular domain (residues 1-219); (2) a 22-amino acid transmembrane domain (residues 220-242); and (3) a 21-amino acid cytoplasmic C-terminal tail (residues 242-263) ((UniProtKB Identifier Number: P13726). The cytoplasmic tail contains two phosphorylation sites at Ser253 and Ser258, and one S-palmitoylation site at Cys245. Deletion or mutation of the cytoplasmic domain was not found to affect tissue factor coagulation activity. Tissue factor has one S-palmitoylation site in the intracellular domain of the protein at Cys245. The Cys245 is located at the amino acid terminus of the intracellular domain and close to the membrane surface. The tissue factor transmembrane domain is composed of a single-spanning α-helix.

The extracellular domain of tissue factor, composed of two fibronectin type III domains, is connected to the transmembrane domain through a six-amino acid linker. This linker provides conformational flexibility to decouple the tissue factor extracellular domain from its transmembrane and cytoplasmic domains. Each tissue factor fibronectin type III module is composed of two overlapping β sheets with the top sheet domain containing three antiparallel β-strands and the bottom sheet containing four β-strands. The β-strands are connected by β-loops between strand βA and βB, βC and βD, and βE and βF, all of which are conserved in conformation in the two modules. There are three short α-helix segments connecting the β-strands. A unique feature of tissue factor is a 17-amino acid β-hairpin between strand β10 and strand β11, which is not a common element of the fibronectin superfamily. The N-terminal domain also contains a 12 amino acid loop between β6F and β7G that is not present in the C-terminal domain and is unique to tissue factor. Such a fibronectin type III domain structure is a feature of the immunoglobulin-like family of protein folds and is conserved among a wide variety of extracellular proteins.

The zymogen FVII is rapidly converted to FVIIa by limited proteolysis once it binds to tissue to form the active tissue factor-FVIIa complex. The FVIIa, which circulates as an enzyme at a concentration of approximately 0.1 nM (1% of plasma FVII), can also bind directly to tissue factor. The allosteric interaction between tissue factor and FVIIa on the tissue factor-FVIIa complex greatly increases the enzymatic activity of FVIIa: an approximate 20- to 100-fold increase in the rate of hydrolysis of small, chromogenic peptidyl substrates, and nearly a million-fold increase in the rate of activation of the natural macromolecular substrates FIX and FX. In concert with allosteric activation of the active site of FVIIa upon binding to tissue factor, the formation of tissue factor-FVIIa complex on phospholipid bilayer (i.e., upon exposure of phosphatidyl-L-serine on membrane surfaces) increases the rate of FIX or FX activation, in a Ca²⁺-dependent manner, an additional 1,000-fold. The roughly million-fold overall increase in FX activation by tissue factor-FVIIa-phospholipid complex relative to free FVIIa is a critical regulatory point for the coagulation cascade.

FVII is a ˜50 kDa, single-chain polypeptide consisting of 406 amino acid residues, with an N-terminal γ-carboxyglutamate-rich (GLA) domain, two epidermal growth factor-like domains (EGF1 and EFG2), and a C-terminal serine protease domain. FVII is activated to FVIIa by a specific proteolytic cleavage of the Ile-¹⁵⁴-Arg¹⁵² bond in the short linker region between the EGF2 and the protease domain. This cleavage results in the light and heavy chains being held together by a single disulfide bond of Cys¹³⁵ and Cys²⁶². FVIIa binds phospholipid membrane in a Ca²⁺-dependent manner through its N-terminal GLA-domain. Immediately C-terminal to the GLA domain is an aromatic stack and two EGF domains. The aromatic stack connects the GLA to EGF1 domain which binds a single Ca²⁺ ion. Occupancy of this Ca²⁺-binding site increases FVIIa amidolytic activity and tissue factor association. The catalytic triad consist of His¹⁹³, Asp²⁴², and Ser³⁴⁴, and binding of a single Ca²⁺ ion within the FVIIa protease domain is critical for its catalytic activity. Proteolytic activation of FVII to FVIIa frees the newly formed amino terminus at Ile¹⁵³ to fold back and be inserted into the activation pocket forming a salt bridge with the carboxylate of Asp³⁴³ to generate the oxyanion hole. Formation of this salt bridge is critical for FVIIa activity. However, oxyanion hole formation does not occur in free FVIIa upon proteolytic activation. As a result, FVIIa circulates in a zymogen-like state that is poorly recognized by plasma protease inhibitors, allowing it to circulate with a half-life of approximately 90 minutes.

Tissue factor-mediated positioning of the FVIIa active site above the membrane surface is important for FVIIa towards cognate substrates. Free FVIIa adopts a stable, extended structure when bound to the membrane with its active site positioned ˜80 Å above the membrane surface. Upon FVIIa binding to tissue factor, the FVa active site is repositioned ˜6 Å closer to the membrane. This modulation may aid in a proper alignment of the FVIIa catalytic triad with the target substrate cleavage site. Using GLA-domainless FVIIa, it has been shown that the active site was still positioned a similar distance above the membrane, demonstrating that tissue factor is able to fully support FVIIa active site positioning even in the absence of FVIIa-membrane interaction. Additional data showed that tissue factor supported full FVIIa proteolytic activity as long as the tissue factor extracellular domain was tethered in some way to the membrane surface. However, raising the active site of FVIIa greater than 80 Å above the membrane surface greatly reduced the ability of the tissue factor-FVIIa complex to activate FX but did not diminish tissue factor-FVIIa amidolytic activity.

Alanine scanning mutagenesis has been used to assess the role of specific amino acid side chains in the tissue factor extracellular domain for interaction with FVIIa (Gibbs et al., Biochemistry 33(47): 14003-14010, 1994; Schullek et al., J Biol Chem 269(30): 19399-19403, 1994). Alanine substitution identified a limited number of residue positions at which alanine replacements cause 5- to 10-fold lower affinity for FVIIa binding. Most of these residue side chains were found to be well-exposed to solvent in the crystal structure, concordant with macromolecular ligand interaction. The FVIIa ligand-binding site is located over an extensive region at the boundary between the two modules. In the C-module, residues Arg¹³⁵ and Phe¹⁴⁰ located on the protruding B-C loop provide an independent contact with FVIIa. Leu¹³³ is located at the base of the fingerlike structure and packed into the cleft between the two modules. This provides continuity to a major cluster of important binding residues consisting of Lys²⁰, Thr⁶⁰, Asp⁵⁸, and Ile²². Thr⁶⁰ is only partially solvent-exposed and may play a local structural role rather than making a significant contact with ligand. The binding site extends onto the concave side of the intermodule angle involving Glu²⁴ and Gln¹¹⁰, and potentially the more distant residue Val²⁰⁷. The binding region extends from Asp⁵⁸ onto a convex surface area formed by Lys⁴⁸, Lys⁴⁶, Gln³⁷, Asp⁴⁴, and Trp⁴⁵. Trp⁴⁵ and Asp⁴⁴ do not interact independently with FVIIa, indicating that the mutational effect at the Trp⁴⁵ position may reflect a structural importance of this side chain for the local packing of the adjacent Asp⁴⁴ and Gln³⁷ side chain. The interactive area further includes two surface-exposed aromatic residues, Phe⁷⁶ and Tyr⁷⁸, which form part of the hydrophobic cluster in the N-module.

The known physiologic substrates of tissue factor-FVIIa are FVII, FIX, and FX and certain proteinase-activated receptors. Mutational analysis has identified a number of residues that, when mutated, support full FVIIa amidolytic activity towards small peptidyl substrates but are deficient in their ability to support macromolecular substrate (i.e., FVII, FIX, and FX) activation (Ruf et al., J Biol Chem 267(31): 22206-22210, 1992; Ruf et al., J Biol Chem 267(9): 6375-6381, 1992; Huang et al., J Biol Chem 271(36): 21752-21757, 1996; Kirchhofer et al., Biochemistry 39(25): 7380-7387, 2000). The tissue factor loop region at residues 159-165, and residues in or adjacent to this flexible loop have been shown to be critical for the proteolytic activity of the tissue factor-FVIIa complex. This defines the proposed substrate-binding exosite region of tissue factor that is quite distant from the FVIIa active site. A substitution of the glycine residue by a marginally bulkier residue alanine, significantly impairs tissue factor-FVIIa proteolytic activity. This suggests that the flexibility afforded by glycine is critical for the loop of residues 159-165 for tissue factor macromolecular substrate recognition.

The residues Lys¹⁶⁵ and Lys¹⁶⁶ have also been demonstrated to be important for substrate recognition and binding. Mutation of either of these residues to alanine results in a significant decrease in the tissue factor co-factor function. Lys¹⁶⁵ and Lys¹⁶⁶ face away from each other, with Lys¹⁶⁵ pointing towards FVIIa in most tissue factor-FVIIa structures, and Lys¹⁶⁶ pointing into the substrate binding exosite region in the crystal structure. Putative salt bridge formation between Lys¹⁶⁵ of and Gla³⁵ of FVIIa would support the notion that tissue factor interaction with the GLA domain of FVIIa modulates substrate recognition. These results suggest that the C-terminal portion of the tissue factor ectodomain directly interacts with the GLA-domain, the possible adjacent EGF1 domains, of FIX and FX, and that the presence of the FVIIa GLA-domain may modulate these interactions either directly or indirectly.

### Soluble Tissue Factor Domain

In some embodiments of any of the polypeptides, compositions, or methods described herein, the soluble tissue factor domain can be a wildtype tissue factor polypeptide lacking the signal sequence, the transmembrane domain, and the intracellular domain. In some examples, the soluble tissue factor domain can be a tissue factor mutant, wherein a wildtype tissue factor polypeptide lacking the signal sequence, the transmembrane domain, and the intracellular domain, and has been further modified at selected amino acids. In some examples, the soluble tissue factor domain can be a soluble human tissue factor domain. In some examples, the soluble tissue factor domain can be a soluble mouse tissue factor domain. In some examples, the soluble tissue factor domain can be a soluble rat tissue factor domain. Non-limiting examples of soluble human tissue factor domains, a mouse soluble tissue factor domain, a rat soluble tissue factor domain, and mutant soluble tissue factor domains are shown below.

Exemplary Soluble Human Tissue Factor Domain
(SEQ ID NO: 1)
SGTTNTVAAYNLTWKSTNFKTILEWEPKPVNQVYTVQISTKSGDWKSKCF
YTTDTECDLTDEIVKDVKQTYLARVFSYPAGNVESTGSAGEPLYENSPEF
TPYLETNLGQPTIQSFEQVGTKVNVTVEDERTLVRRNNTFLSLRDVFGKD
LIYTLYYWKSSSSGKKTAKTNTNEFLIDVDKGENYCFSVQAVIPSRTVNR
KSTDSPVECMGQEKGEFRE
Exemplary Nucleic Acid Encoding Soluble Human
Tissue Factor Domain
(SEQ ID NO: 36)
AGCGGCACAACCAACACAGTCGCTGCCTATAACCTCACTTGGAAGAGCAC
CAACTTCAAAACCATCCTCGAATGGGAACCCAAACCCGTTAACCAAGTTT
ACACCGTGCAGATCAGCACCAAGTCCGGCGACTGGAAGTCCAAATGTTTC
TATACCACCGACACCGAGTGCGATCTCACCGATGAGATCGTGAAAGATGT
GAAACAGACCTACCTCGCCCGGGTGTTTAGCTACCCCGCCGGCAATGTGG
AGAGCACTGGTTCCGCTGGCGAGCCTTTATACGAGAACAGCCCCGAATTT
ACCCCTTACCTCGAGACCAATTTAGGACAGCCCACCATCCAAAGCTTTGA
GCAAGTTGGCACAAAGGTGAATGTGACAGTGGAGGACGAGCGGACTTTAG
TGCGGCGGAACAACACCTTTCTCAGCCTCCGGGATGTGTTCGGCAAAGAT
TTAATCTACACACTGTATTACTGGAAGTCCTCTTCCTCCGGCAAGAAGAC
AGCTAAAACCAACACAAACGAGTTTTTAATCGACGTGGATAAAGGCGAAA
ACTACTGTTTCAGCGTGCAAGCTGTGATCCCCTCCCGGACCGTGAATAGG
AAAAGCACCGATAGCCCCGTTGAGTGCATGGGCCAAGAAAAGGGCGAGTT
CCGGGAG
Exemplary Mutant Soluble Human Tissue Factor
Domain
(SEQ ID NO: 37)
SGTTNTVAAYNLTWKSTNFATALEWEPKPVNQVYTVQISTKSGDWKSKCF
YTTDTECALTDEIVKDVKQTYLARVFSYPAGNVESTGSAGEPLYENSPEF
TPYLETNLGQPTIQSFEQVGTKVNVTVEDERTLVARNNTALSLRDVFGKD
LIYTLYYWKSSSSGKKTAKTNTNEFLIDVDKGENYCFSVQAVIPSRTVNR
KSTDSPVECMGQEKGEFRE
Exemplary Mutant Soluble Human Tissue Factor
Domain
(SEQ ID NO: 38)
SGTTNTVAAYNLTWKSTNFATALEWEPKPVNQVYTVQISTKSGDAKSKCF
YTTDTECALTDEIVKDVKQTYLARVFSYPAGNVESTGSAGEPLAENSPEF
TPYLETNLGQPTIQSFEQVGTKVNVTVEDERTLVARNNTALSLRDVFGKD
LIYTLYYWKSSSSGKKTAKTNTNEFLIDVDKGENYCFSVQAVIPSRTVNR
KSTDSPVECMGQEKGEFRE
Exemplary Soluble Mouse Tissue Factor Domain
(SEQ ID NO: 39)
Agipekafnltwistdfktilewqpkptnytytvqisdrsrnwknkcfst
tdtecdltdeivkdvtwayeakvlsvprrnsvhgdgdqlvihgeeppftn
apkflpyrdtnlgqpviqqfeqdgrklnvvvkdsltlvrkngtfltlrqv
fgkdlgyiityrkgsstgkktnitntnefsidveegvsycffvqamifsr
ktnqnspgsstvcteqwksflge
Exemplary Soluble Rat Tissue Factor Domain
(SEQ ID NO: 40)
agtppgkafnltwistdfktilewqpkptnytytvqisdrsrnwkykctg
ttdtecdltdeivkdvnwtyearvlsvpwrnsthgketlfgthgeeppft
narkflpyrdtkigqpviqkyeqggtklkvtvkdsftlvrkngtfltlrq
vfgndlgyiltyrkdsstgrktntthtneflidvekgvsycffaqavifs
rktnhkspesitkcteqwksvlge

In some embodiments, a soluble tissue factor domain can include a sequence that is at least 70% identical, at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to SEQ ID NO: 1, 37, 38, 39, or 40. In some embodiments, a soluble tissue factor domain can include a sequence of SEQ ID NO: 1, 37, 38, 39, or 40, with one to twenty amino acids (e.g., 2, 3, 4, 5, 6, 7, 8, 9, 10, 11, 12, 13, 14, 15, 16, 17, 18, 19, or 20) amino acids removed from its N-terminus and/or one to twenty amino acids (e.g., 2, 3, 4, 5, 6, 7, 8, 9, 10, 11, 12, 13, 14, 15, 16, 17, 18, 19, or 20) amino acids removed from its C-terminus.

As can be appreciated in the art, one skilled in the art would understand that mutation of amino acids that are conserved between different mammalian species is more likely to decrease the activity and/or structural stability of the protein, while mutation of amino acids that are not conserved between different mammalian species is less likely to decrease the activity and/or structural stability of the protein.

In some examples of any of the multi-chain chimeric polypeptides described herein, the soluble tissue factor domain is not capable of binding to Factor VIIa. In some examples of any of the multi-chain chimeric polypeptides described herein, the soluble tissue factor domain does not convert inactive Factor X into Factor Xa. In some embodiments of any of the multi-chain chimeric polypeptides described herein, the multi-chain chimeric polypeptide does not stimulate blood coagulation in a mammal.

In some examples, the soluble tissue factor domain can be a soluble human tissue factor domain. In some embodiments, the soluble tissue factor domain can be a soluble mouse tissue factor domain. In some embodiments, the soluble tissue factor domain can be a soluble rat tissue factor domain.

In some examples, the soluble tissue factor domain does not include one or more (e.g., two, three, four, five, six, or seven) of: a lysine at an amino acid position that corresponds to amino acid position 20 of mature wildtype human tissue factor protein; an isoleucine at an amino acid position that corresponds to amino acid position 22 of mature wildtype human tissue factor protein; a tryptophan at an amino acid position that corresponds to amino acid position 45 of mature wildtype human tissue factor protein; an aspartic acid at an amino acid position that corresponds to amino acid position 58 of mature wildtype human tissue factor protein; a tyrosine at an amino acid position that corresponds to amino acid position 94 of mature wildtype human tissue factor protein; an arginine at an amino acid position that corresponds to amino acid position 135 of mature wildtype human tissue factor protein; and a phenylalanine at an amino acid position that corresponds to amino acid position 140 of mature wildtype human tissue factor protein. In some embodiments, the mutant soluble tissue factor possesses the amino acid sequence of SEQ ID NO: 37 or SEQ ID NO: 38.

In some examples, the soluble tissue factor domain can be encoded by a nucleic acid including a sequence that is at least 70% identical, at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to SEQ ID NO: 36.

In some embodiments, the soluble tissue factor domain can have a total length of about 20 amino acids to about 220 amino acids, about 20 amino acids to about 215 amino acids, about 20 amino acids to about 210 amino acids, about 20 amino acids to about 205 amino acids, about 20 amino acids to about 200 amino acids, about 20 amino acids to about 195 amino acids, about 20 amino acids to about 190 amino acids, about 20 amino acids to about 185 amino acids, about 20 amino acids to about 180 amino acids, about 20 amino acids to about 175 amino acids, about 20 amino acids to about 170 amino acids, about 20 amino acids to about 165 amino acids, about 20 amino acids to about 160 amino acids, about 20 amino acids to about 155 amino acids, about 20 amino acids to about 150 amino acids, about 20 amino acids to about 145 amino acids, about 20 amino acids to about 140 amino acids, about 20 amino acids to about 135 amino acids, about 20 amino acids to about 130 amino acids, about 20 amino acids to about 125 amino acids, about 20 amino acids to about 120 amino acids, about 20 amino acids to about 115 amino acids, about 20 amino acids to about 110 amino acids, about 20 amino acids to about 105 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 60 amino acids, about 20 amino acids to about 50 amino acids, about 20 amino acids to about 40 amino acids, about 20 amino acids to about 30 amino acids, about 30 amino acids to about 220 amino acids, about 30 amino acids to about 215 amino acids, about 30 amino acids to about 210 amino acids, about 30 amino acids to about 205 amino acids, about 30 amino acids to about 200 amino acids, about 30 amino acids to about 195 amino acids, about 30 amino acids to about 190 amino acids, about 30 amino acids to about 185 amino acids, about 30 amino acids to about 180 amino acids, about 30 amino acids to about 175 amino acids, about 30 amino acids to about 170 amino acids, about 30 amino acids to about 165 amino acids, about 30 amino acids to about 160 amino acids, about 30 amino acids to about 155 amino acids, about 30 amino acids to about 150 amino acids, about 30 amino acids to about 145 amino acids, about 30 amino acids to about 140 amino acids, about 30 amino acids to about 135 amino acids, about 30 amino acids to about 130 amino acids, about 30 amino acids to about 125 amino acids, about 30 amino acids to about 120 amino acids, about 30 amino acids to about 115 amino acids, about 30 amino acids to about 110 amino acids, about 30 amino acids to about 105 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 60 amino acids, about 30 amino acids to about 50 amino acids, about 30 amino acids to about 40 amino acids, about 40 amino acids to about 220 amino acids, about 40 amino acids to about 215 amino acids, about 40 amino acids to about 210 amino acids, about 40 amino acids to about 205 amino acids, about 40 amino acids to about 200 amino acids, about 40 amino acids to about 195 amino acids, about 40 amino acids to about 190 amino acids, about 40 amino acids to about 185 amino acids, about 40 amino acids to about 180 amino acids, about 40 amino acids to about 175 amino acids, about 40 amino acids to about 170 amino acids, about 40 amino acids to about 165 amino acids, about 40 amino acids to about 160 amino acids, about 40 amino acids to about 155 amino acids, about 40 amino acids to about 150 amino acids, about 40 amino acids to about 145 amino acids, about 40 amino acids to about 140 amino acids, about 40 amino acids to about 135 amino acids, about 40 amino acids to about 130 amino acids, about 40 amino acids to about 125 amino acids, about 40 amino acids to about 120 amino acids, about 40 amino acids to about 115 amino acids, about 40 amino acids to about 110 amino acids, about 40 amino acids to about 105 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 60 amino acids, about 40 amino acids to about 50 amino acids, about 50 amino acids to about 220 amino acids, about 50 amino acids to about 215 amino acids, about 50 amino acids to about 210 amino acids, about 50 amino acids to about 205 amino acids, about 50 amino acids to about 200 amino acids, about 50 amino acids to about 195 amino acids, about 50 amino acids to about 190 amino acids, about 50 amino acids to about 185 amino acids, about 50 amino acids to about 180 amino acids, about 50 amino acids to about 175 amino acids, about 50 amino acids to about 170 amino acids, about 50 amino acids to about 165 amino acids, about 50 amino acids to about 160 amino acids, about 50 amino acids to about 155 amino acids, about 50 amino acids to about 150 amino acids, about 50 amino acids to about 145 amino acids, about 50 amino acids to about 140 amino acids, about 50 amino acids to about 135 amino acids, about 50 amino acids to about 130 amino acids, about 50 amino acids to about 125 amino acids, about 50 amino acids to about 120 amino acids, about 50 amino acids to about 115 amino acids, about 50 amino acids to about 110 amino acids, about 50 amino acids to about 105 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 60 amino acids, about 60 amino acids to about 220 amino acids, about 60 amino acids to about 215 amino acids, about 60 amino acids to about 210 amino acids, about 60 amino acids to about 205 amino acids, about 60 amino acids to about 200 amino acids, about 60 amino acids to about 195 amino acids, about 60 amino acids to about 190 amino acids, about 60 amino acids to about 185 amino acids, about 60 amino acids to about 180 amino acids, about 60 amino acids to about 175 amino acids, about 60 amino acids to about 170 amino acids, about 60 amino acids to about 165 amino acids, about 60 amino acids to about 160 amino acids, about 60 amino acids to about 155 amino acids, about 60 amino acids to about 150 amino acids, about 60 amino acids to about 145 amino acids, about 60 amino acids to about 140 amino acids, about 60 amino acids to about 135 amino acids, about 60 amino acids to about 130 amino acids, about 60 amino acids to about 125 amino acids, about 60 amino acids to about 120 amino acids, about 60 amino acids to about 115 amino acids, about 60 amino acids to about 110 amino acids, about 60 amino acids to about 105 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 70 amino acids to about 220 amino acids, about 70 amino acids to about 215 amino acids, about 70 amino acids to about 210 amino acids, about 70 amino acids to about 205 amino acids, about 70 amino acids to about 200 amino acids, about 70 amino acids to about 195 amino acids, about 70 amino acids to about 190 amino acids, about 70 amino acids to about 185 amino acids, about 70 amino acids to about 180 amino acids, about 70 amino acids to about 175 amino acids, about 70 amino acids to about 170 amino acids, about 70 amino acids to about 165 amino acids, about 70 amino acids to about 160 amino acids, about 70 amino acids to about 155 amino acids, about 70 amino acids to about 150 amino acids, about 70 amino acids to about 145 amino acids, about 70 amino acids to about 140 amino acids, about 70 amino acids to about 135 amino acids, about 70 amino acids to about 130 amino acids, about 70 amino acids to about 125 amino acids, about 70 amino acids to about 120 amino acids, about 70 amino acids to about 115 amino acids, about 70 amino acids to about 110 amino acids, about 70 amino acids to about 105 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 80 amino acids to about 220 amino acids, about 80 amino acids to about 215 amino acids, about 80 amino acids to about 210 amino acids, about 80 amino acids to about 205 amino acids, about 80 amino acids to about 200 amino acids, about 80 amino acids to about 195 amino acids, about 80 amino acids to about 190 amino acids, about 80 amino acids to about 185 amino acids, about 80 amino acids to about 180 amino acids, about 80 amino acids to about 175 amino acids, about 80 amino acids to about 170 amino acids, about 80 amino acids to about 165 amino acids, about 80 amino acids to about 160 amino acids, about 80 amino acids to about 155 amino acids, about 80 amino acids to about 150 amino acids, about 80 amino acids to about 145 amino acids, about 80 amino acids to about 140 amino acids, about 80 amino acids to about 135 amino acids, about 80 amino acids to about 130 amino acids, about 80 amino acids to about 125 amino acids, about 80 amino acids to about 120 amino acids, about 80 amino acids to about 115 amino acids, about 80 amino acids to about 110 amino acids, about 80 amino acids to about 105 amino acids, about 80 amino acids to about 100 amino acids, about 80 amino acids to about 95 amino acids, about 80 amino acids to about 90 amino acids, about 90 amino acids to about 220 amino acids, about 90 amino acids to about 215 amino acids, about 90 amino acids to about 210 amino acids, about 90 amino acids to about 205 amino acids, about 90 amino acids to about 200 amino acids, about 90 amino acids to about 195 amino acids, about 90 amino acids to about 190 amino acids, about 90 amino acids to about 185 amino acids, about 90 amino acids to about 180 amino acids, about 90 amino acids to about 175 amino acids, about 90 amino acids to about 170 amino acids, about 90 amino acids to about 165 amino acids, about 90 amino acids to about 160 amino acids, about 90 amino acids to about 155 amino acids, about 90 amino acids to about 150 amino acids, about 90 amino acids to about 145 amino acids, about 90 amino acids to about 140 amino acids, about 90 amino acids to about 135 amino acids, about 90 amino acids to about 130 amino acids, about 90 amino acids to about 125 amino acids, about 90 amino acids to about 120 amino acids, about 90 amino acids to about 115 amino acids, about 90 amino acids to about 110 amino acids, about 90 amino acids to about 105 amino acids, about 90 amino acids to about 100 amino acids, about 100 amino acids to about 220 amino acids, about 100 amino acids to about 215 amino acids, about 100 amino acids to about 210 amino acids, about 100 amino acids to about 205 amino acids, about 100 amino acids to about 200 amino acids, about 100 amino acids to about 195 amino acids, about 100 amino acids to about 190 amino acids, about 100 amino acids to about 185 amino acids, about 100 amino acids to about 180 amino acids, about 100 amino acids to about 175 amino acids, about 100 amino acids to about 170 amino acids, about 100 amino acids to about 165 amino acids, about 100 amino acids to about 160 amino acids, about 100 amino acids to about 155 amino acids, about 100 amino acids to about 150 amino acids, about 100 amino acids to about 145 amino acids, about 100 amino acids to about 140 amino acids, about 100 amino acids to about 135 amino acids, about 100 amino acids to about 130 amino acids, about 100 amino acids to about 125 amino acids, about 100 amino acids to about 120 amino acids, about 100 amino acids to about 115 amino acids, about 100 amino acids to about 110 amino acids, about 110 amino acids to about 220 amino acids, about 110 amino acids to about 215 amino acids, about 110 amino acids to about 210 amino acids, about 110 amino acids to about 205 amino acids, about 110 amino acids to about 200 amino acids, about 110 amino acids to about 195 amino acids, about 110 amino acids to about 190 amino acids, about 110 amino acids to about 185 amino acids, about 110 amino acids to about 180 amino acids, about 110 amino acids to about 175 amino acids, about 110 amino acids to about 170 amino acids, about 110 amino acids to about 165 amino acids, about 110 amino acids to about 160 amino acids, about 110 amino acids to about 155 amino acids, about 110 amino acids to about 150 amino acids, about 110 amino acids to about 145 amino acids, about 110 amino acids to about 140 amino acids, about 110 amino acids to about 135 amino acids, about 110 amino acids to about 130 amino acids, about 110 amino acids to about 125 amino acids, about 110 amino acids to about 120 amino acids, about 110 amino acids to about 115 amino acids, about 115 amino acids to about 220 amino acids, about 115 amino acids to about 215 amino acids, about 115 amino acids to about 210 amino acids, about 115 amino acids to about 205 amino acids, about 115 amino acids to about 200 amino acids, about 115 amino acids to about 195 amino acids, about 115 amino acids to about 190 amino acids, about 115 amino acids to about 185 amino acids, about 115 amino acids to about 180 amino acids, about 115 amino acids to about 175 amino acids, about 115 amino acids to about 170 amino acids, about 115 amino acids to about 165 amino acids, about 115 amino acids to about 160 amino acids, about 115 amino acids to about 155 amino acids, about 115 amino acids to about 150 amino acids, about 115 amino acids to about 145 amino acids, about 115 amino acids to about 140 amino acids, about 115 amino acids to about 135 amino acids, about 115 amino acids to about 130 amino acids, about 115 amino acids to about 125 amino acids, about 115 amino acids to about 120 amino acids, about 120 amino acids to about 220 amino acids, about 120 amino acids to about 215 amino acids, about 120 amino acids to about 210 amino acids, about 120 amino acids to about 205 amino acids, about 120 amino acids to about 200 amino acids, about 120 amino acids to about 195 amino acids, about 120 amino acids to about 190 amino acids, about 120 amino acids to about 185 amino acids, about 120 amino acids to about 180 amino acids, about 120 amino acids to about 175 amino acids, about 120 amino acids to about 170 amino acids, about 120 amino acids to about 165 amino acids, about 120 amino acids to about 160 amino acids, about 120 amino acids to about 155 amino acids, about 120 amino acids to about 150 amino acids, about 120 amino acids to about 145 amino acids, about 120 amino acids to about 140 amino acids, about 120 amino acids to about 135 amino acids, about 120 amino acids to about 130 amino acids, about 120 amino acids to about 125 amino acids, about 125 amino acids to about 220 amino acids, about 125 amino acids to about 215 amino acids, about 125 amino acids to about 210 amino acids, about 125 amino acids to about 205 amino acids, about 125 amino acids to about 200 amino acids, about 125 amino acids to about 195 amino acids, about 125 amino acids to about 190 amino acids, about 125 amino acids to about 185 amino acids, about 125 amino acids to about 180 amino acids, about 125 amino acids to about 175 amino acids, about 125 amino acids to about 170 amino acids, about 125 amino acids to about 165 amino acids, about 125 amino acids to about 160 amino acids, about 125 amino acids to about 155 amino acids, about 125 amino acids to about 150 amino acids, about 125 amino acids to about 145 amino acids, about 125 amino acids to about 140 amino acids, about 125 amino acids to about 135 amino acids, about 125 amino acids to about 130 amino acids, about 130 amino acids to about 220 amino acids, about 130 amino acids to about 215 amino acids, about 130 amino acids to about 210 amino acids, about 130 amino acids to about 205 amino acids, about 130 amino acids to about 200 amino acids, about 130 amino acids to about 195 amino acids, about 130 amino acids to about 190 amino acids, about 130 amino acids to about 185 amino acids, about 130 amino acids to about 180 amino acids, about 130 amino acids to about 175 amino acids, about 130 amino acids to about 170 amino acids, about 130 amino acids to about 165 amino acids, about 130 amino acids to about 160 amino acids, about 130 amino acids to about 155 amino acids, about 130 amino acids to about 150 amino acids, about 130 amino acids to about 145 amino acids, about 130 amino acids to about 140 amino acids, about 130 amino acids to about 135 amino acids, about 135 amino acids to about 220 amino acids, about 135 amino acids to about 215 amino acids, about 135 amino acids to about 210 amino acids, about 135 amino acids to about 205 amino acids, about 135 amino acids to about 200 amino acids, about 135 amino acids to about 195 amino acids, about 135 amino acids to about 190 amino acids, about 135 amino acids to about 185 amino acids, about 135 amino acids to about 180 amino acids, about 135 amino acids to about 175 amino acids, about 135 amino acids to about 170 amino acids, about 135 amino acids to about 165 amino acids, about 135 amino acids to about 160 amino acids, about 135 amino acids to about 155 amino acids, about 135 amino acids to about 150 amino acids, about 135 amino acids to about 145 amino acids, about 135 amino acids to about 140 amino acids, about 140 amino acids to about 220 amino acids, about 140 amino acids to about 215 amino acids, about 140 amino acids to about 210 amino acids, about 140 amino acids to about 205 amino acids, about 140 amino acids to about 200 amino acids, about 140 amino acids to about 195 amino acids, about 140 amino acids to about 190 amino acids, about 140 amino acids to about 185 amino acids, about 140 amino acids to about 180 amino acids, about 140 amino acids to about 175 amino acids, about 140 amino acids to about 170 amino acids, about 140 amino acids to about 165 amino acids, about 140 amino acids to about 160 amino acids, about 140 amino acids to about 155 amino acids, about 140 amino acids to about 150 amino acids, about 140 amino acids to about 145 amino acids, about 145 amino acids to about 220 amino acids, about 145 amino acids to about 215 amino acids, about 145 amino acids to about 210 amino acids, about 145 amino acids to about 205 amino acids, about 145 amino acids to about 200 amino acids, about 145 amino acids to about 195 amino acids, about 145 amino acids to about 190 amino acids, about 145 amino acids to about 185 amino acids, about 145 amino acids to about 180 amino acids, about 145 amino acids to about 175 amino acids, about 145 amino acids to about 170 amino acids, about 145 amino acids to about 165 amino acids, about 145 amino acids to about 160 amino acids, about 145 amino acids to about 155 amino acids, about 145 amino acids to about 150 amino acids, about 150 amino acids to about 220 amino acids, about 150 amino acids to about 215 amino acids, about 150 amino acids to about 210 amino acids, about 150 amino acids to about 205 amino acids, about 150 amino acids to about 200 amino acids, about 150 amino acids to about 195 amino acids, about 150 amino acids to about 190 amino acids, about 150 amino acids to about 185 amino acids, about 150 amino acids to about 180 amino acids, about 150 amino acids to about 175 amino acids, about 150 amino acids to about 170 amino acids, about 150 amino acids to about 165 amino acids, about 150 amino acids to about 160 amino acids, about 150 amino acids to about 155 amino acids, about 155 amino acids to about 220 amino acids, about 155 amino acids to about 215 amino acids, about 155 amino acids to about 210 amino acids, about 155 amino acids to about 205 amino acids, about 155 amino acids to about 200 amino acids, about 155 amino acids to about 195 amino acids, about 155 amino acids to about 190 amino acids, about 155 amino acids to about 185 amino acids, about 155 amino acids to about 180 amino acids, about 155 amino acids to about 175 amino acids, about 155 amino acids to about 170 amino acids, about 155 amino acids to about 165 amino acids, about 155 amino acids to about 160 amino acids, about 160 amino acids to about 220 amino acids, about 160 amino acids to about 215 amino acids, about 160 amino acids to about 210 amino acids, about 160 amino acids to about 205 amino acids, about 160 amino acids to about 200 amino acids, about 160 amino acids to about 195 amino acids, about 160 amino acids to about 190 amino acids, about 160 amino acids to about 185 amino acids, about 160 amino acids to about 180 amino acids, about 160 amino acids to about 175 amino acids, about 160 amino acids to about 170 amino acids, about 160 amino acids to about 165 amino acids, about 165 amino acids to about 220 amino acids, about 165 amino acids to about 215 amino acids, about 165 amino acids to about 210 amino acids, about 165 amino acids to about 205 amino acids, about 165 amino acids to about 200 amino acids, about 165 amino acids to about 195 amino acids, about 165 amino acids to about 190 amino acids, about 165 amino acids to about 185 amino acids, about 165 amino acids to about 180 amino acids, about 165 amino acids to about 175 amino acids, about 165 amino acids to about 170 amino acids, about 170 amino acids to about 220 amino acids, about 170 amino acids to about 215 amino acids, about 170 amino acids to about 210 amino acids, about 170 amino acids to about 205 amino acids, about 170 amino acids to about 200 amino acids, about 170 amino acids to about 195 amino acids, about 170 amino acids to about 190 amino acids, about 170 amino acids to about 185 amino acids, about 170 amino acids to about 180 amino acids, about 170 amino acids to about 175 amino acids, about 175 amino acids to about 220 amino acids, about 175 amino acids to about 215 amino acids, about 175 amino acids to about 210 amino acids, about 175 amino acids to about 205 amino acids, about 175 amino acids to about 200 amino acids, about 175 amino acids to about 195 amino acids, about 175 amino acids to about 190 amino acids, about 175 amino acids to about 185 amino acids, about 175 amino acids to about 180 amino acids, about 180 amino acids to about 220 amino acids, about 180 amino acids to about 215 amino acids, about 180 amino acids to about 210 amino acids, about 180 amino acids to about 205 amino acids, about 180 amino acids to about 200 amino acids, about 180 amino acids to about 195 amino acids, about 180 amino acids to about 190 amino acids, about 180 amino acids to about 185 amino acids, about 185 amino acids to about 220 amino acids, about 185 amino acids to about 215 amino acids, about 185 amino acids to about 210 amino acids, about 185 amino acids to about 205 amino acids, about 185 amino acids to about 200 amino acids, about 185 amino acids to about 195 amino acids, about 185 amino acids to about 190 amino acids, about 190 amino acids to about 220 amino acids, about 190 amino acids to about 215 amino acids, about 190 amino acids to about 210 amino acids, about 190 amino acids to about 205 amino acids, about 190 amino acids to about 200 amino acids, about 190 amino acids to about 195 amino acids, about 195 amino acids to about 220 amino acids, about 195 amino acids to about 215 amino acids, about 195 amino acids to about 210 amino acids, about 195 amino acids to about 205 amino acids, about 195 amino acids to about 200 amino acids, about 200 amino acids to about 220 amino acids, about 200 amino acids to about 215 amino acids, about 200 amino acids to about 210 amino acids, about 200 amino acids to about 205 amino acids, about 205 amino acids to about 220 amino acids, about 205 amino acids to about 215 amino acids, about 205 amino acids to about 210 amino acids, about 210 amino acids to about 220 amino acids, about 210 amino acids to about 215 amino acids, or about 215 amino acids to about 220 amino acids.

### Linker Sequences

In some embodiments, the linker sequence can be a flexible linker sequence. Non-limiting examples of linker sequences that can be used are described in Klein et al., Protein Engineering, Design & Selection 27(10):325-330, 2014; Priyanka et al., Protein Sci. 22(2):153-167, 2013. In some examples, the linker sequence is a synthetic linker sequence.

In some embodiments of any of the multi-chain chimeric polypeptides described herein, the first chimeric polypeptide can include one, two, three, four, five, six, seven, eight, nine, or ten linker sequence(s) (e.g., the same or different linker sequences, e.g., any of the exemplary linker sequences described herein or known in the art). In some embodiments of any of the multi-chain chimeric polypeptides described herein, the second chimeric polypeptide can include one, two, three, four, five, six, seven, eight, nine, or ten linker sequence(s) (e.g., the same or different linker sequences, e.g., any of the exemplary linker sequences described herein or known in the art).

In some embodiments, a linker sequence can have a total length of 1 amino acid to about 100 amino acids, 1 amino acid to about 90 amino acids, 1 amino acid to about 80 amino acids, 1 amino acid to about 70 amino acids, 1 amino acid to about 60 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 24 amino acids, 1 amino acid to about 22 amino acids, 1 amino acid to about 20 amino acids, 1 amino acid to about 18 amino acids, 1 amino acid to about 16 amino acids, 1 amino acid to about 14 amino acids, 1 amino acid to about 12 amino acids, 1 amino acid to about 10 amino acids, 1 amino acid to about 8 amino acids, 1 amino acid to about 6 amino acids, 1 amino acid to about 4 amino acids, about 2 amino acids to about 100 amino acids, about 2 amino acids to about 90 amino acids, about 2 amino acids to about 80 amino acids, about 2 amino acids to about 70 amino acids, about 2 amino acids to about 60 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 4 amino acids, about 4 amino acids to about 100 amino acids, about 4 amino acids to about 90 amino acids, about 4 amino acids to about 80 amino acids, about 4 amino acids to about 70 amino acids, about 4 amino acids to about 60 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 6 amino acids to about 100 amino acids, about 6 amino acids to about 90 amino acids, about 6 amino acids to about 80 amino acids, about 6 amino acids to about 70 amino acids, about 6 amino acids to about 60 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 14 amino acids, about 6 amino acids to about 12 amino acids, about 6 amino acids to about 10 amino acids, about 6 amino acids to about 8 amino acids, about 8 amino acids to about 100 amino acids, about 8 amino acids to about 90 amino acids, about 8 amino acids to about 80 amino acids, about 8 amino acids to about 70 amino acids, about 8 amino acids to about 60 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 14 amino acids, about 8 amino acids to about 12 amino acids, about 8 amino acids to about 10 amino acids, about 10 amino acids to about 100 amino acids, about 10 amino acids to about 90 amino acids, about 10 amino acids to about 80 amino acids, about 10 amino acids to about 70 amino acids, about 10 amino acids to about 60 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 14 amino acids, about 10 amino acids to about 12 amino acids, about 12 amino acids to about 100 amino acids, about 12 amino acids to about 90 amino acids, about 12 amino acids to about 80 amino acids, about 12 amino acids to about 70 amino acids, about 12 amino acids to about 60 amino acids, about 12 amino acids to about 50 amino acids, about 12 amino acids to about 45 amino acids, about 12 amino acids to about 40 amino acids, about 12 amino acids to about 35 amino acids, about 12 amino acids to about 30 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 14 amino acids, about 14 amino acids to about 100 amino acids, about 14 amino acids to about 90 amino acids, about 14 amino acids to about 80 amino acids, about 14 amino acids to about 70 amino acids, about 14 amino acids to about 60 amino acids, about 14 amino acids to about 50 amino acids, about 14 amino acids to about 45 amino acids, about 14 amino acids to about 40 amino acids, about 14 amino acids to about 35 amino acids, about 14 amino acids to about 30 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 16 amino acids to about 100 amino acids, about 16 amino acids to about 90 amino acids, about 16 amino acids to about 80 amino acids, about 16 amino acids to about 70 amino acids, about 16 amino acids to about 60 amino acids, about 16 amino acids to about 50 amino acids, about 16 amino acids to about 45 amino acids, about 16 amino acids to about 40 amino acids, about 16 amino acids to about 35 amino acids, about 16 amino acids to about 30 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 100 amino acids, about 18 amino acids to about 90 amino acids, about 18 amino acids to about 80 amino acids, about 18 amino acids to about 70 amino acids, about 18 amino acids to about 60 amino acids, about 18 amino acids to about 50 amino acids, about 18 amino acids to about 45 amino acids, about 18 amino acids to about 40 amino acids, about 18 amino acids to about 35 amino acids, about 18 amino acids to about 30 amino acids, about 18 amino acids to about 25 amino acids, about 18 amino acids to about 24 amino acids, about 18 amino acids to about 22 amino acids, about 18 amino acids to about 20 amino acids, about 20 amino acids to about 100 amino acids, about 20 amino acids to about 90 amino acids, about 20 amino acids to about 80 amino acids, about 20 amino acids to about 70 amino acids, about 20 amino acids to about 60 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 20 amino acids to about 24 amino acids, about 20 amino acids to about 22 amino acids, about 22 amino acids to about 100 amino acids, about 22 amino acids to about 90 amino acids, about 22 amino acids to about 80 amino acids, about 22 amino acids to about 70 amino acids, about 22 amino acids to about 60 amino acids, about 22 amino acids to about 50 amino acids, about 22 amino acids to about 45 amino acids, about 22 amino acids to about 40 amino acids, about 22 amino acids to about 35 amino acids, about 22 amino acids to about 30 amino acids, about 22 amino acids to about 25 amino acids, about 22 amino acids to about 24 amino acids, about 25 amino acids to about 100 amino acids, about 25 amino acids to about 90 amino acids, about 25 amino acids to about 80 amino acids, about 25 amino acids to about 70 amino acids, about 25 amino acids to about 60 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 100 amino acids, about 30 amino acids to about 90 amino acids, about 30 amino acids to about 80 amino acids, about 30 amino acids to about 70 amino acids, about 30 amino acids to about 60 amino acids, about 30 amino acids to about 50 amino acids, about 30 amino acids to about 45 amino acids, about 30 amino acids to about 40 amino acids, about 30 amino acids to about 35 amino acids, about 35 amino acids to about 100 amino acids, about 35 amino acids to about 90 amino acids, about 35 amino acids to about 80 amino acids, about 35 amino acids to about 70 amino acids, about 35 amino acids to about 60 amino acids, about 35 amino acids to about 50 amino acids, about 35 amino acids to about 45 amino acids, about 35 amino acids to about 40 amino acids, about 40 amino acids to about 100 amino acids, about 40 amino acids to about 90 amino acids, about 40 amino acids to about 80 amino acids, about 40 amino acids to about 70 amino acids, about 40 amino acids to about 60 amino acids, about 40 amino acids to about 50 amino acids, about 40 amino acids to about 45 amino acids, about 45 amino acids to about 100 amino acids, about 45 amino acids to about 90 amino acids, about 45 amino acids to about 80 amino acids, about 45 amino acids to about 70 amino acids, about 45 amino acids to about 60 amino acids, about 45 amino acids to about 50 amino acids, about 50 amino acids to about 100 amino acids, about 50 amino acids to about 90 amino acids, about 50 amino acids to about 80 amino acids, about 50 amino acids to about 70 amino acids, about 50 amino acids to about 60 amino acids, about 60 amino acids to about 100 amino acids, about 60 amino acids to about 90 amino acids, about 60 amino acids to about 80 amino acids, about 60 amino acids to about 70 amino acids, about 70 amino acids to about 100 amino acids, about 70 amino acids to about 90 amino acids, about 70 amino acids to about 80 amino acids, about 80 amino acids to about 100 amino acids, about 80 amino acids to about 90 amino acids, or about 90 amino acids to about 100 amino acids.

In some embodiments, the linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS), e.g., 1, 2, 3, 4, 5, 6, 7, 8, 9, or 10 or more GS pairs. In some embodiments, the linker has one or more Gly-Gly-Gly-Ser (GGGS) sequences, e.g., 1, 2, 3, 4, 5, 6, 7, 8, 9, or 10 or more GGGS sequences. In some embodiments, the linker has one or more Gly-Gly-Gly-Gly-Ser (GGGGS) sequences, e.g., 1, 2, 3, 4, 5, 6, 7, 8, 9, or 10 or more GGGGS sequences. In some embodiments, the linker has one or more Gly-Gly-Ser-Gly (GGSG) sequences, e.g., 1, 2, 3, 4, 5, 6, 7, 8, 9, or 10 or more GGSG sequences.

In some embodiments, the linker sequence can comprise or consist of GGGGSGGGGSGGGGS (SEQ ID NO: 5). In some embodiments, the linker sequence can be encoded by a nucleic acid comprising or consisting of:

(SEQ ID NO: 41)
GGCGGTGGAGGATCCGGAGGAGGTGGCTCCGGCGGCGGAGGATCT.

In some embodiments, the linker sequence can comprise or consist of: GGGSGGGS.

### Exemplary First and Second Target-Binding Domains

In some embodiments of any of the multi-chain chimeric polypeptides described herein, the first target-binding domain and the second target-binding domain bind specifically to the same antigen. In some embodiments of these multi-chain chimeric polypeptides, the first target-binding domain and the second target-binding domain bind specifically to the same epitope. In some embodiments of these multi-chain chimeric polypeptides, the first target-binding domain and the second target-binding domain include the same amino acid sequence.

In some embodiments of any of the multi-chain chimeric polypeptides described herein, the first target-binding domain and the second target-binding domain bind specifically to different antigens.

In some embodiments of any of the multi-chain chimeric polypeptides described herein, one or both of the first target-binding domain and the second target-binding domain is an antigen-binding domain. In some embodiments of any of the multi-chain chimeric polypeptides described herein, the first target-binding domain and the second target-binding domain are each antigen-binding domains.

In some embodiments of any of the multi-chain chimeric polypeptides described herein, the antigen-binding domain includes or is a scFv or a single domain antibody (e.g., a VHH or a VNAR domain).

In some examples, an antigen-binding domain (e.g., any of the antigen-binding domains described herein) can bind specifically to a ligand of TGF-βRII (see, e.g., antigen-binding domains that can bind specifically to TGF-β described in US 2021/0061897, US 2020/0399358, US 2020/0392221, US 2019/0315850, US 2019/0092846, US 2021/0403545, US 2021/0355204, and US 2019/0177406, each of which is herein incorporated by reference).

The antigen-binding domains present in any of the multi-chain chimeric polypeptides described herein are each independently selected from the group consisting of: a VHH domain, a VNAR domain, and a scFv. In some embodiments, any of the antigen-binding domains described herein is a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv. Additional examples of antigen-binding domains that can be used in any of the multi-chain chimeric polypeptide are known in the art.

A VHH domain is a single monomeric variable antibody domain that can be found in camelids. A VNAR domain is a single monomeric variable antibody domain that can be found in cartilaginous fish. Non-limiting aspects of VHH domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments, the antigen-binding domains in the multi-chain chimeric polypeptides described herein are both VHH domains, or at least one antigen-binding domain is a VHH domain. In some embodiments, the antigen-binding domains in the multi-chain chimeric polypeptides described herein are both VNAR domains, or at least one antigen-binding domain is a VNAR domain. In some embodiments, the antigen-binding domains in the multi-chain chimeric polypeptides described herein are both scFv domains, or at least one antigen-binding domain is a scFv domain.

In some embodiments, two or more of polypeptides present in the multi-chain chimeric polypeptide can assemble (e.g., non-covalently assemble) to form any of the antigen-binding domains described herein, e.g., an antigen-binding fragment of an antibody (e.g., any of the antigen-binding fragments of an antibody described herein), a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a Kk-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv₂, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, and a scFv1-PEG-scFv2. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

An “Tv” fragment includes a non-covalently-linked dimer of one heavy chain variable domain and one light chain variable domain.

A “Fab” fragment includes the constant domain of the light chain and the first constant domain (C_{H1}) of the heavy chain, in addition to the heavy and light chain variable domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

In some embodiments of any of the antigen-binding domains described herein can bind to an antigen selected from the group consisting of: a protein, a carbohydrate, a lipid, and a combination thereof.

Additional examples and aspects of antigen-binding domains are known in the art.

In some embodiments of any of the multi-chain chimeric polypeptides described herein, one or both of the first target-binding domain and the second target-binding domain is a soluble interleukin receptor, a soluble cytokine receptor, or a ligand receptor. In some embodiments, the soluble receptor is a soluble TGF-β receptor II (TGF-β RII) (see, e.g., those described in Yung et al., Am. J. Resp. Crit. Care Med. 194(9):1140-1151, 2016) or a soluble TGF-βRIII (see, e.g., those described in Heng et al., Placenta 57:320, 2017). In some embodiments, one or both of the first target-binding domain and the second target-binding domain is a soluble TGF-β receptor II (TGF-βRII). In some embodiments, the first target-binding domain and the second target-binding domain are a soluble TGF-βRII.

In some embodiments, the first target-binding domain comprises a first sequence that is at least 80%, at least 85%, at least 90%, at least 92%, at least 94%, at least 95%, at least 96%, at least 98%, or at least 99% identical to SEQ ID NO: 2 and a second sequence that is at least 80%, at least 85%, at least 90%, at least 92%, at least 94%, at least 95%, at least 96%, at least 98%, or at least 99% identical to SEQ ID NO: 2, wherein the first and second sequence are separated by a linker (e.g., any of the exemplary linkers described herein, e.g., SEQ ID NO: 5). In some embodiments, the first target-binding domain comprises a first sequence of SEQ ID NO: 2 and a second sequence of SEQ ID NO: 2.

In some embodiments, the first target-binding domain comprises a sequence that is at least 80%, at least 85%, at least 90%, at least 92%, at least 94%, at least 95%, at least 96%, at least 98%, or at least 99% identical to SEQ ID NO: 6. In some embodiments, the first target-binding domain comprises a sequence of SEQ ID NO: 6.

In some embodiments, the second target-binding domain comprises a first sequence that is at least 80%, at least 85%, at least 90%, at least 92%, at least 94%, at least 95%, at least 96%, at least 98%, or at least 99% identical to SEQ ID NO: 2 and a second sequence that is at least 80%, at least 85%, at least 90%, at least 92%, at least 94%, at least 95%, at least 96%, at least 98%, or at least 99% identical to SEQ ID NO: 2, wherein the first and second sequence are separated by a linker (e.g., any of the exemplary linkers described herein, e.g., SEQ ID NO: 5). In some embodiments, the second target-binding domain comprises a first sequence of SEQ ID NO: 2 and a second sequence of SEQ ID NO: 2.

In some embodiments, the second target-binding domain comprises a sequence that is at least 80%, at least 85%, at least 90%, at least 92%, at least 94%, at least 95%, at least 96%, at least 98%, or at least 99% identical to SEQ ID NO: 6. In some embodiments, the second target-binding domain comprises a sequence of SEQ ID NO: 6.

Additional examples of soluble interleukin receptors and soluble cytokine receptors are known in the art.

### Exemplary First and Second Target-Binding Domains

In some embodiments, the first sequence of the first target-binding domain comprises an asparagine, a glutamine, a cysteine, or a tyrosine at amino acid position 32 in SEQ ID NO: 20. In some embodiments, the first sequence of the first target-binding domain comprises an asparagine at amino acid position 32 in SEQ ID NO: 20.

In some embodiments, the first sequence of the first target-binding domain comprises an alanine, a glycine, or a valine at amino acid position 119 in SEQ ID NO: 20. In some embodiments, the first sequence of the first target-binding domain comprises an alanine at amino acid position 119 in SEQ ID NO: 20.

In some embodiments, the first sequence of the first target-binding domain comprises an asparagine, a glutamine, a cysteine, or a tyrosine at amino acid position 32 and an alanine, a glycine, or a valine at amino acid position 119 in SEQ ID NO: 20. In some embodiments, the first sequence of the first target-binding domain comprises an asparagine at amino acid position 32 and an alanine at amino acid position 119 in SEQ ID NO: 20.

In some embodiments, the second sequence of the first target-binding domain comprises an asparagine, a glutamine, a cysteine, or a tyrosine at amino acid position 32 in SEQ ID NO: 20.

In some embodiments, the second sequence of the first target-binding domain comprises an asparagine at amino acid position 32 in SEQ ID NO: 20.

In some embodiments, the second sequence of the first target-binding domain comprises an alanine, a glycine, or a valine at amino acid position 119 in SEQ ID NO: 20. In some embodiments, the second sequence of the first target-binding domain comprises an alanine at amino acid position 119 in SEQ ID NO: 20.

In some embodiments, the second sequence of the first target-binding domain comprises an asparagine, a glutamine, a cysteine, or a tyrosine at amino acid position 32 and an alanine, a glycine, or a valine at amino acid position 119 in SEQ ID NO: 20. In some embodiments, the second sequence of the first target-binding domain comprises an asparagine at amino acid position 32 and an alanine at amino acid position 119 in SEQ ID NO: 20.

In some embodiments, the first and second sequence of the first target-binding domain are separated by a linker (e.g., any of the exemplary linkers described herein, e.g., SEQ ID NO: 5).

In some embodiments, the first sequence of the second target-binding domain comprises an asparagine, a glutamine, a cysteine, or a tyrosine at amino acid position 32 in SEQ ID NO: 20. In some embodiments, the first sequence of the second target-binding domain comprises an asparagine at amino acid position 32 in SEQ ID NO: 20.

In some embodiments, the first sequence of the second target-binding domain comprises an alanine, a glycine, or a valine at amino acid position 119 in SEQ ID NO: 20. In some embodiments, the first sequence of the second target-binding domain comprises an alanine at amino acid position 119 in SEQ ID NO: 20.

In some embodiments, the first sequence of the second target-binding domain comprises an asparagine, a glutamine, a cysteine, or a tyrosine at amino acid position 32 and an alanine, a glycine, or a valine at amino acid position 119 in SEQ ID NO: 20. In some embodiments, the first sequence of the second target-binding domain comprises an asparagine at amino acid position 32 and an alanine at amino acid position 119 in SEQ ID NO: 20.

In some embodiments, the second sequence of the second target-binding domain comprises an asparagine, a glutamine, a cysteine, or a tyrosine at amino acid position 32 in SEQ ID NO: 20.

In some embodiments, the second sequence of the second target-binding domain comprises an asparagine at amino acid position 32 in SEQ ID NO: 20.

In some embodiments, the second sequence of the second target-binding domain comprises an alanine, a glycine, or a valine at amino acid position 119 in SEQ ID NO: 20. In some embodiments, the second sequence of the second target-binding domain comprises an alanine at amino acid position 119 in SEQ ID NO: 20.

In some embodiments, the second sequence of the second target-binding domain comprises an asparagine, a glutamine, a cysteine, or a tyrosine at amino acid position 32 and an alanine, a glycine, or a valine at amino acid position 119 in SEQ ID NO: 20. In some embodiments, the second sequence of the second target-binding domain comprises an asparagine at amino acid position 32 and an alanine at amino acid position 119 in SEQ ID NO: 20.

In some embodiments, the first and second sequence of the first target-binding domain are separated by a linker (e.g., any of the exemplary linkers described herein, e.g., SEQ ID NO: 5).

Exemplary First or Second Sequence
(SEQ ID NO: 20)
IPPHVQKSVNNDMIVTDNNGAVKFPQLCKFCNVRFSTCDNQKSCMSNCSI
TSICEKPQEVCVAVWRKNDENITLETVCHDPKLPYHDFILEDAASPKCIM
KEKKKPGETFFMCSCSSDACNDNIIFSEEYNTSNPD

In some embodiments, the first and/or second sequence of the first and/or second target-binding domain comprises a sequence at least 80% identical, least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical to SEQ ID NO: 20.

In some embodiments, the first and/or second sequence of the first and/or second target-binding domain is encoded by a nucleic acid comprising a sequence at least 80% identical, at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical or 100% identical to any one of SEQ ID NOs: 21, 22, 23, and 24.

Exemplary Nucleic Acid Encoding an Exemplary
First or Second Sequence
(SEQ ID NO: 21)
ATCCCCCCCCATGTGCAAAAGAGCGTGAACAACGATATGATCGTGACCGA
CAACAACGGCGCCGTGAAGTTTCCCCAGCTCTGCAAGTTCTGCAACGTCA
GGTTCAGCACCTGCGATAATCAGAAGTCCTGCATGTCCAACTGCAGCATC
ACCTCCATCTGCGAGAAGCCCCAAGAAGTGTGCGTGGCCGTGTGGCGGAA
AAATGACGAGAACATCACCCTGGAGACCGTGTGTCACGACCCCAAGCTCC
CTTATCACGACTTCATTCTGGAGGACGCTGCCTCCCCCAAATGCATCATG
AAGGAGAAGAAGAAGCCCGGAGAGACCTTCTTTATGTGTTCCTGTAGCAG
CGACGCCTGTAACGACAACATCATCTTCAGCGAAGAGTACAACACCAGCA
ACCCTGAT
Exemplary Nucleic Acid Encoding an Exemplary
First or Second Sequence
(SEQ ID NO: 22)
ATTCCTCCCCACGTGCAGAAGAGCGTGAATAATGACATGATCGTGACCGA
TAACAATGGCGCCGTGAAATTTCCCCAGCTGTGCAAATTCTGCAATGTGA
GGTTTTCCACCTGCGACAACCAGAAGTCCTGTATGAGCAACTGCTCCATC
ACCTCCATCTGTGAGAAGCCTCAGGAGGTGTGCGTGGCTGTCTGGCGGAA
GAATGACGAGAATATCACCCTGGAAACCGTCTGCCACGATCCCAAGCTGC
CCTACCACGATTTCATCCTGGAAGACGCCGCCAGCCCTAAGTGCATCATG
AAAGAGAAAAAGAAGCCTGGCGAGACCTTTTTCATGTGCTCCTGCAGCAG
CGACGCTTGCAACGACAATATCATCTTTAGCGAGGAATACAATACCAGCA
ACCCCGAC
Exemplary Nucleic Acid Encoding an Exemplary
First or Second Sequence
(SEQ ID NO: 23)
ATCCCACCTCACGTGCAGAAAAGCGTCAATAATGACATGATCGTGACTGA
CAATAACGGCGCCGTCAAGTTTCCACAGCTGTGTAAGTTCTGCAACGTGA
GATTTTCCACATGCGACAACCAGAAGAGCTGTATGAGCAACTGCAGCATC
ACAAGCATCTGCGAGAAGCCACAAGAGGTCTGCGTGGCCGTCTGGAGAAA
GAACGACGAGAACATCACTCTCGAGACTGTGTGTCACGACCCTAAGCTCC
CATACCATGACTTCATCCTCGAGGATGCTGCCTCCCCTAAATGCATTATG
AAGGAGAAAAAAAAGCCCGGCGAGACATTCTTTATGTGCAGCTGCTCCTC
CGACGCCTGCAACGACAACATCATCTTTAGCGAAGAATACAACACTAGCA
ACCCAGAT
Exemplary Nucleic Acid Encoding an Exemplary
First or Second Sequence
(SEQ ID NO: 24
ATCCCACCACACGTGCAGAAGTCCGTCAACAACGACATGATTGTGACTGA
CAACAACGGCGCCGTGAAGTTCCCACAACTCTGCAAGTTTTGCAATGTGA
GGTTCTCCACATGTGACAACCAGAAAAGCTGCATGTCCAACTGCTCCATC
ACTAGCATCTGTGAGAAACCTCAAGAGGTCTGTGTGGCTGTGTGGAGGAA
GAACGATGAGAACATCACACTCGAGACAGTCTGCCACGACCCAAAGCTGC
CATATCACGACTTCATTCTCGAGGACGCCGCCAGCCCTAAGTGCATCATG
AAGGAGAAGAAGAAGCCCGGCGAGACATTTTTCATGTGTAGCTGCAGCTC
CGATGCCTGTAACGACAATATTATCTTTAGCGAGGAGTATAACACATCCA
ATCCAGAC

In some embodiments, the first and/or second target-binding domain comprises a sequence that is at least 80% identical, at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical or 100% identical to SEQ ID NO: 25.

Exemplary First and/or Sequence Target-Binding
Domain
(SEQ ID NO: 25)
IPPHVQKSVNNDMIVTDNNGAVKFPQLCKFCNVRFSTCDNQKSCMSNCSI
TSICEKPQEVCVAVWRKNDENITLETVCHDPKLPYHDFILEDAASPKCIM
KEKKKPGETFFMCSCSSDACNDNIIFSEEYNTSNPDGGGGSGGGGSGGGG
SIPPHVQKSVNNDMIVTDNNGAVKFPQLCKFCNVRFSTCDNQKSCMSNCS
ITSICEKPQEVCVAVWRKNDENITLETVCHDPKLPYHDFILEDAASPKCI
MKEKKKPGETFFMCSCSSDACNDNIIFSEEYNTSNPD

In some embodiments, the first and/or second target-binding domain are encoded by nucleic acid that is at least 80% identical, at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical or 100% identical to SEQ ID NO: 26 or SEQ ID NO: 27.

Exemplary Sequence Encoding Exemplary First
and/or Second Target-Binding Domain
(SEQ ID NO: 26)
ATCCCCCCCCATGTGCAAAAGAGCGTGAACAACGATATGATCGTGACCGA
CAACAACGGCGCCGTGAAGTTTCCCCAGCTCTGCAAGTTCTGCAACGTCA
GGTTCAGCACCTGCGATAATCAGAAGTCCTGCATGTCCAACTGCAGCATC
ACCTCCATCTGCGAGAAGCCCCAAGAAGTGTGCGTGGCCGTGTGGCGGAA
AAATGACGAGAACATCACCCTGGAGACCGTGTGTCACGACCCCAAGCTCC
CTTATCACGACTTCATTCTGGAGGACGCTGCCTCCCCCAAATGCATCATG
AAGGAGAAGAAGAAGCCCGGAGAGACCTTCTTTATGTGTTCCTGTAGCAG
CGACGCCTGTAACGACAACATCATCTTCAGCGAAGAGTACAACACCAGCA
ACCCTGATGGAGGTGGCGGATCCGGAGGTGGAGGTTCTGGTGGAGGTGGG
AGTATTCCTCCCCACGTGCAGAAGAGCGTGAATAATGACATGATCGTGAC
CGATAACAATGGCGCCGTGAAATTTCCCCAGCTGTGCAAATTCTGCAATG
TGAGGTTTTCCACCTGCGACAACCAGAAGTCCTGTATGAGCAACTGCTCC
ATCACCTCCATCTGTGAGAAGCCTCAGGAGGTGTGCGTGGCTGTCTGGCG
GAAGAATGACGAGAATATCACCCTGGAAACCGTCTGCCACGATCCCAAGC
TGCCCTACCACGATTTCATCCTGGAAGACGCCGCCAGCCCTAAGTGCATC
ATGAAAGAGAAAAAGAAGCCTGGCGAGACCTTTTTCATGTGCTCCTGCAG
CAGCGACGCTTGCAACGACAATATCATCTTTAGCGAGGAATACAATACCA
GCAACCCCGAC
Exemplary Sequence Encoding Exemplary First and/or
Second Target-Binding Domain
(SEQ ID NO: 27)
ATCCCACCTCACGTGCAGAAAAGCGTCAATAATGACATGATCGTGACTGA
CAATAACGGCGCCGTCAAGTTTCCACAGCTGTGTAAGTTCTGCAACGTGA
GATTTTCCACATGCGACAACCAGAAGAGCTGTATGAGCAACTGCAGCATC
ACAAGCATCTGCGAGAAGCCACAAGAGGTCTGCGTGGCCGTCTGGAGAAA
GAACGACGAGAACATCACTCTCGAGACTGTGTGTCACGACCCTAAGCTCC
CATACCATGACTTCATCCTCGAGGATGCTGCCTCCCCTAAATGCATTATG
AAGGAGAAAAAAAAGCCCGGCGAGACATTCTTTATGTGCAGCTGCTCCTC
CGACGCCTGCAACGACAACATCATCTTTAGCGAAGAATACAACACTAGCA
ACCCAGATGGCGGCGGAGGATCCGGAGGAGGAGGCTCCGGAGGCGGAGGC
AGCATCCCACCACACGTGCAGAAGTCCGTCAACAACGACATGATTGTGAC
TGACAACAACGGCGCCGTGAAGTTCCCACAACTCTGCAAGTTTTGCAATG
TGAGGTTCTCCACATGTGACAACCAGAAAAGCTGCATGTCCAACTGCTCC
ATCACTAGCATCTGTGAGAAACCTCAAGAGGTCTGTGTGGCTGTGTGGAG
GAAGAACGATGAGAACATCACACTCGAGACAGTCTGCCACGACCCAAAGC
TGCCATATCACGACTTCATTCTCGAGGACGCCGCCAGCCCTAAGTGCATC
ATGAAGGAGAAGAAGAAGCCCGGCGAGACATTTTTCATGTGTAGCTGCAG
CTCCGATGCCTGTAACGACAATATTATCTTTAGCGAGGAGTATAACACAT
CCAATCCAGAC

### Additional Target-Binding Domains

In some embodiments of any of the multi-chain chimeric polypeptides, the first chimeric polypeptide further includes one or more (e.g., two, three, four, five, six, seven, eight, nine, or ten) additional target-binding domain(s) (e.g., any of the exemplary target-binding domains described herein or known in the art), where at least one of the one or more additional antigen-binding domain(s) is positioned between the soluble tissue factor domain (e.g., any of the exemplary soluble tissue factor domains described herein or known in the art) and the first domain of the pair of affinity domains (e.g., any of the exemplary first domains of any of the exemplary pairs of affinity domains described herein). In some embodiments, the first chimeric polypeptide can further include a linker sequence (e.g., any of the exemplary linker sequences described herein or known in the art) between the soluble tissue factor domain (e.g., any of the exemplary soluble tissue factor domains described herein) and the at least one of the one or more additional target-binding domain(s) (e.g., any of the exemplary target-binding domains described herein or known in the art), and/or a linker sequence (e.g., any of the exemplary linker sequences described herein or known in the art) between the at least one of the one or more additional target-binding domain(s) (e.g., any of the exemplary target-binding domains described herein or known in the art) and the first domain of the pair of affinity domains (e.g., any of the exemplary first domains described herein of any of the exemplary pairs of affinity domains described herein).

In some embodiments of any of the multi-chain chimeric polypeptides described herein, the first chimeric polypeptide further includes one or more (e.g., two, three, four, five, six, seven, eight, nine, or ten) additional target-binding domains at the N-terminal and/or C-terminal end of the first chimeric polypeptide. In some embodiments, at least one of the one or more additional target-binding domains (e.g., any of the exemplary target-binding domains described herein or known in the art) directly abuts the first domain of the pair of affinity domains (e.g., any of the exemplary first domains described herein of any of the exemplary pairs of affinity domains described herein) in the first chimeric polypeptide. In some embodiments, the first chimeric polypeptide further includes a linker sequence (e.g., any of the exemplary linker sequences described herein or known in the art) between the at least one of the one or more additional target-binding domains (e.g., any of the exemplary target-binding domains described herein or known in the art) and the first domain of the pair of affinity domains (e.g., any of the exemplary first domains described herein of any of the exemplary pairs of affinity domains described herein). In some embodiments, the at least one of the one or more additional target-binding domains (e.g., any of the exemplary target-binding domains described herein or known in the art) directly abuts the first target-binding domain (e.g., any of the exemplary target-binding domains described herein or known in the art) in the first chimeric polypeptide. In some embodiments, the first chimeric polypeptide further comprises a linker sequence (e.g., any of the exemplary linker sequences described herein or known in the art) between the at least one of the one or more additional target-binding domains (e.g., any of the exemplary target-binding domains described herein or known in the art) and the first target-binding domain (e.g., any of the exemplary target-binding domains described herein or known in the art).

In some embodiments of any of the multi-chain chimeric polypeptides described herein, at least one of the one or more additional target-binding domains (e.g., any of the exemplary target-binding domains described herein or known in the art) is disposed at the N- and/or C-terminus of the first chimeric polypeptide, and at least one of the one or more additional target-binding domains (e.g., any of the exemplary target-binding domains described herein or known in the art) is positioned between the soluble tissue factor domain (e.g., any of the exemplary soluble tissue factor domains described herein or known in the art) and the first domain of the pair of affinity domains (e.g., any of the exemplary first domains of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the N-terminus directly abuts the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the first chimeric polypeptide further comprises a linker sequence (e.g., any of the linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the C-terminus directly abuts the first target-binding domain (e.g., any of the exemplary target-binding domains described herein or known in the art) or the first domain of the pair of affinity domains (e.g., any of the exemplary first domains of any of the exemplary pairs of affinity domains described herein) in the first chimeric polypeptide. In some embodiments, the first chimeric polypeptide further includes a linker sequence (e.g., any of the exemplary linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one of the one or more additional target-binding domains (e.g., any of the exemplary target-binding domains described herein or known in the art) positioned between the soluble tissue factor domain (e.g., any of the exemplary soluble tissue factor domains described herein) and the first domain of the pair of affinity domains (e.g., any of the first domains described herein or any of the exemplary pairs of affinity domains described herein), directly abuts the soluble tissue factor domain and/or the first domain of the pair of affinity domains. In some embodiments, the first chimeric polypeptide further comprises a linker sequence (e.g., any of the exemplary linker sequences described herein or known in the art) disposed (i) between the soluble tissue factor domain (e.g., any of the exemplary soluble tissue factor domains described herein) and the at least one of the one or more additional target-binding domains (e.g., any of the exemplary target-binding domains described herein or known in the art) positioned between the soluble tissue factor domain (e.g., any of the exemplary soluble tissue factor domains described herein) and the first domain of the pair of affinity domains (e.g., any of the exemplary first domains of any of the exemplary pairs of affinity domains described herein), and/or (ii) between the first domain of the pair of affinity domains and the at least one of the one or more additional target-binding domains positioned between the soluble tissue factor domain and the first domain of the pair of affinity domains.

In some embodiments of any of the multi-chain chimeric polypeptides described herein, the second chimeric polypeptide further includes one or more (e.g., two, three, four, five, six, seven, eight, nine, or ten) additional target-binding domains (e.g., any of the exemplary target-binding domains described herein or known in the art) at the N-terminal end and/or the C-terminal end of the second chimeric polypeptide. In some embodiments, at least one of the one or more additional target-binding domains (e.g., any of the exemplary target-binding domains described herein or known in the art) directly abuts the second domain of the pair of affinity domains (e.g., any of the exemplary second domains of any of the exemplary pairs of affinity domains described herein) in the second chimeric polypeptide.

In some embodiments, the second chimeric polypeptide further includes a linker sequence (e.g., any of the exemplary linker sequences described herein or known in the art) between at least one of the one or more additional target-binding domains (e.g., any of the exemplary target-binding domains described herein or known in the art) and the second domain of the pair of affinity domains (e.g., any of the second domains described herein of any of the exemplary pairs of affinity domains described herein) in the second chimeric polypeptide. In some embodiments, at least one of the one or more additional target-binding domains (e.g., any of the exemplary target-binding domains described herein or known in the art) directly abuts the second target-binding domain (e.g., any of the target-binding domains described herein or known in the art) in the second chimeric polypeptide. In some embodiments, the second chimeric polypeptide further includes a linker sequence (e.g., any of the exemplary linker sequences described herein or known in the art) between at least one of the one or more additional target-binding domains (e.g., any of the exemplary target binding domains described herein or known in the art) and the second target-binding domain (e.g., any of the exemplary target binding domains described herein or known in the art) in the second chimeric polypeptide.

In some embodiments of any of the multi-chain chimeric polypeptides described herein, two or more (e.g., three or more, four or more, five or more, six or more, seven or more, eight or more, nine or more, or ten or more) of the first target-binding domain, the second target-binding domain, and the one or more additional target-binding domains bind specifically to the same antigen. In some embodiments, two or more (e.g., three or more, four or more, five or more, six or more, seven or more, eight or more, nine or more, or ten or more) of the first target-binding domain, the second target-binding domain, and the one or more additional target-binding domains bind specifically to the same epitope. In some embodiments, two or more (e.g., three or more, four or more, five or more, six or more, seven or more, eight or more, nine or more, or ten or more) of the first target-binding domain, the second target-binding domain, and the one or more additional target-binding domains include the same amino acid sequence. In some embodiments, the first target-binding domain, the second target-binding domain, and the one or more additional target-binding domains each bind specifically to the same antigen. In some embodiments, the first target-binding domain, the second target-binding domain, and the one or more additional target-binding domains each bind specifically to the same epitope. In some embodiments, the first target-binding domain, the second target-binding domain, and the one or more additional target-binding domains each include the same amino acid sequence.

In some embodiments of any of the multi-chain chimeric polypeptides described herein, the first target-binding domain, the second target-binding domain, and the one or more additional target-binding domains bind specifically to different antigens. In some embodiments of any of the multi-chain chimeric polypeptides described herein, one or more (e.g., two or more, three or more, four or more, five or more, six or more, seven or more, eight or more, nine or more, or ten or more) of the first target-binding domain, the second target-binding domain, and the one or more target-binding domains is an antigen-binding domain. In some embodiments, the first target-binding domain, the second target-binding domain, and the one or more additional target-binding domains are each an antigen-binding domain (e.g., a scFv or a single-domain antibody).

### Pairs of Affinity Domains

In some embodiments, a multi-chain chimeric polypeptide includes: 1) a first chimeric polypeptide that includes a first domain of a pair of affinity domains, and 2) a second chimeric polypeptide that includes a second domain of a pair of affinity domains such that the first chimeric polypeptide and the second chimeric polypeptide associate through the binding of the first domain and the second domain of the pair of affinity domains. In some embodiments, the pair of affinity domains is a sushi domain from an alpha chain of human IL-15 receptor (IL15Rα) and a soluble IL-15. A sushi domain, also known as a short consensus repeat or type 1 glycoprotein motif, is a common motif in protein-protein interaction. Sushi domains have been identified on a number of protein-binding molecules, including complement components Cr, C1s, factor H, and C2m, as well as the nonimmunologic molecules factor XIII and β2-glycoprotein. A typical Sushi domain has approximately 60 amino acid residues and contains four cysteines (Ranganathan, Pac. Symp Biocomput. 2000:155-67). The first cysteine can form a disulfide bond with the third cysteine, and the second cysteine can form a disulfide bridge with the fourth cysteine. In some embodiments in which one member of the pair of affinity domains is a soluble IL-15, the soluble IL15 has a D8N or D8A amino acid substitution. In some embodiments in which one member of the pair of affinity domains is an alpha chain of human IL-15 receptor (IL11Rα), the human IL15Rα is a mature full-length IL15Rα. In some embodiments, the pair of affinity domains is barnase and barnstar. In some embodiments, the pair of affinity domains is a PKA and an AKAP. In some embodiments, the pair of affinity domains is an adapter/docking tag module based on mutated RNase I fragments (Rossi, Proc Natl Acad Sci USA. 103:6841-6846, 2006; Sharkey et al., Cancer Res. 68:5282-5290, 2008; Rossi et al., Trends Pharmacol Sci. 33:474-481, 2012) or SNARE modules based on interactions of the proteins syntaxin, synaptotagmin, synaptobrevin, and SNAP25 (Deyev et al., Nat Biotechnol. 1486-1492, 2003).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains and the second domain of the pair of affinity domains bind to each other with a dissociation equilibrium constant (K_{D}) of less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the first domain of the pair of affinity domains and the second domain of the pair of affinity domains bind to each other with a K_{D} of about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰ M, about 1×10⁻⁹ M to about 1×10⁻¹¹ M, about 1×10⁻¹⁰ M to about 1×10⁻¹² M, about 1×10⁻¹¹ M to about 1×10⁻¹³ M, about 1×10⁻⁴ M to about 1×10⁻⁵ M, about 1×10⁻⁵ M to about 1×10⁻⁶ M, about 1×10⁻⁶ M to about 1×10⁻⁷ M, about 1×10⁻⁷ M to about 1×10⁻⁸ M, about 1×10⁻⁸ M to about 1×10⁻⁹ M, about 1×10⁻⁹ M to about 1×10⁻¹⁰ M, about 1×10⁻¹⁰ M to about 1×10⁻¹¹ M, about 1×10⁻¹¹ M to about 1×10⁻¹² M, or about 1×10⁻¹² M to about 1×10⁻¹³ M (inclusive). Any of a variety of different methods known in the art can be used to determine the K_{D} value of the binding of the first domain of the pair of affinity domains and the second domain of the pair of affinity domains (e.g., an electrophoretic mobility shift assay, a filter binding assay, surface plasmon resonance, and a biomolecular binding kinetics assay, etc.).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains, the second domain of the pair of affinity domains, or both is about 10 to 100 amino acids in length. For example, a first domain of a pair of affinity domains, a second domain of a pair of affinity domains, or both can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a first domain of a pair of affinity domains, a second domain of a pair of affinity domains, or both is about 10, 15, 20, 25, 30, 35, 40, 45, 50, 55, 60, 65, 70, 75, 80, 85, 90, 95, or 100 amino acids in length.

In some embodiments, any of the first and/or second domains of a pair of affinity domains disclosed herein can include one or more additional amino acids (e.g., 1, 2, 3, 5, 6, 7, 8, 9, 10, or more amino acids) at its N-terminus and/or C-terminus, so long as the function of the first and/or second domains of a pair of affinity domains remains intact. For example, a sushi domain from an alpha chain of human IL-15 receptor (IL15Rα) can include one or more additional amino acids at the N-terminus and/or the C-terminus, while still retaining the ability to bind to a soluble IL-15. Additionally or alternatively, a soluble IL-15 can include one or more additional amino acids at the N-terminus and/or the C-terminus, while still retaining the ability to bind to a sushi domain from an alpha chain of human IL-15 receptor (IL15Rα).

A non-limiting example of a sushi domain from an alpha chain of IL-15 receptor alpha (IL15Rα) can include a sequence that is at least 70% identical, at least 75% identical, at least 80% identical, at least 85% identical, at least 90% identical, at least 95% identical, at least 99% identical, or 100% identical to ITCPPPMSVEHADIWVKSYSLYSRERYICNSGFKRKAGTSSLTECVLNKATNVAHWTTPS LKCIR (SEQ ID NO: 42). In some embodiments, a sushi domain from an alpha chain of IL15Rα can be encoded by a nucleic acid including

(SEQ ID NO: 43)
ATTACATGCCCCCCTCCCATGAGCGTGGAGCACGCCGACATCTGGGTGAA
GAGCTATAGCCTCTACAGCCGGGAGAGGTATATCTGTAACAGCGGCTTCA
AGAGGAAGGCCGGCACCAGCAGCCTCACCGAGTGCGTGCTGAATAAGGCT
ACCAACGTGGCTCACTGGACAACACCCTCTTTAAAGTGCATCCGG.

In some embodiments, a soluble IL-15 can include a sequence that is at least 70% identical, at least 75% identical, at least 80% identical, at least 85% identical, at least 90% identical, at least 95% identical, at least 99% identical, or 100% identical to NWVNVISDLKKIEDLIQSMHIDATLYTESDVHPSCKVTAMKCFLLELQVISLESGDASIHD TVENLIILANNSLSSNGNVTESGCKECEELEEKNIKEFLQSFVHIVQMFINTS (SEQ ID NO: 44). In some embodiments, a soluble IL-15 can be encoded by a nucleic acid including the sequence of

(SEQ ID NO: 45)
AACTGGGTGAACGTCATCAGCGATTTAAAGAAGATCGAAGATTTAATTCA
GTCCATGCATATCGACGCCACTTTATACACAGAATCCGACGTGCACCCCT
CTTGTAAGGTGACCGCCATGAAATGTTTTTTACTGGAGCTGCAAGTTATC
TCTTTAGAGAGCGGAGACGCTAGCATCCACGACACCGTGGAGAATTTAAT
CATTTTAGCCAATAACTCTTTATCCAGCAACGGCAACGTGACAGAGTCCG
GCTGCAAGGAGTGCGAAGAGCTGGAGGAGAAGAACATCAAGGAGTTTCTG
CAATCCTTTGTGCACATTGTCCAGATGTTCATCAATACCTCC.

In some embodiments, a soluble IL-15 can include a D8N amino acid substitution. In some embodiments, the soluble IL-15 with D8N mutant (IL15D8N) can include a sequence that is at least 70% identical, at least 75% identical, at least 80% identical, at least 85% identical, at least 90% identical, at least 92% identical, at least 94% identical, at least 95% identical, at least 96% identical, at least 98% identical, at least 99% identical, or 100% identical to NWVNVISNLKKIEDLIQ SMHIDATLYTESDVHPSCKVTAMKCFLLELQVISLESGDASIIID TVENLIILANNSLSSNGNVTESGCKECEELEEKNIKEFLQSFVHIVQMFINTS (SEQ ID NO: 46). In some embodiments, the soluble IL-15 with D8N mutant (IL15D8N) can be encoded by a nucleic acid including the sequence of:

(SEQ ID NO: 47)
AACTGGGTGAATGTAATAAGTAATTTGAAAAAAATTGAAGATCTTATTC
AATCTATGCATATTGATGCTACTTTATATACGGAAAGTGATGTTCACCC
CAGTTGCAAAGTAACAGCAATGAAGTGCTTTCTCTTGGAGTTACAAGTT
ATTTCACTTGAGTCCGGAGATGCAAGTATTCATGATACAGTAGAAAATC
TGATCATCCTAGCAAACAACAGTTTGTCTTCTAATGGGAATGTAACAGA
ATCTGGATGCAAAGAATGTGAGGAACTGGAGGAAAAAAATATTAAAGAA
TTTTTGCAGAGTTTTGTACATATTGTCCAAATGTTCATCAACACTTCT.

### Signal Sequence

In some embodiments, a multi-chain chimeric polypeptide includes a first chimeric polypeptide that includes a signal sequence at its N-terminal end. In some embodiments, a multi-chain chimeric polypeptide includes a second chimeric polypeptide that includes a signal sequence at its N-terminal end. In some embodiments, both the first chimeric polypeptide of a multi-chain chimeric polypeptide and a second chimeric polypeptide of the multi-chain chimeric polypeptide include a signal sequence. As will be understood by those of ordinary skill in the art, a signal sequence is an amino acid sequence that is present at the N-terminus of a number of endogenously produced proteins that directs the protein to the secretory pathway (e.g., the protein is directed to reside in certain intracellular organelles, to reside in the cell membrane, or to be secreted from the cell). Signal sequences are heterogeneous and differ greatly in their primary amino acid sequences. However, signal sequences are typically 16 to 30 amino acids in length and include a hydrophilic, usually positively charged N-terminal region, a central hydrophobic domain, and a C-terminal region that contains the cleavage site for signal peptidase.

In some embodiments, a first chimeric polypeptide of a multi-chain chimeric polypeptide, a second chimeric polypeptide of the multi-chain chimeric polypeptide, or both includes a signal sequence having an amino acid sequence MKWVTFISLLFLFSSAYS (SEQ ID NO: 48). In some embodiments, a first chimeric polypeptide of a multi-chain chimeric polypeptide, a second chimeric polypeptide of the multi-chain chimeric polypeptide, or both includes a signal sequence encoded by the nucleic acid sequence:

(SEQ ID NO: 49)
ATGAAATGGGTGACCTTTATTTCTTTACTGTTCCTCTTTAGCAGCGCCT
ACTCC,
(SEQ ID NO: 50)
ATGAAGTGGGTCACATTTATCTCTTTACTGTTCCTCTTCTCCAGCGCCT
ACAGC,
or
(SEQ ID NO: 51)
ATGAAATGGGTGACCTTTATTTCTTTACTGTTCCTCTTTAGCAGCGCCT
ACTCC.

In some embodiments, a first chimeric polypeptide of a multi-chain chimeric polypeptide, a second chimeric polypeptide of the multi-chain chimeric polypeptide, or both includes a signal sequence having an amino acid sequence MKCLLYLAFLFLGVNC (SEQ ID NO: 52). In some embodiments, a first chimeric polypeptide of a multi-chain chimeric polypeptide, a second chimeric polypeptide of the multi-chain chimeric polypeptide, or both includes a signal sequence having an amino acid sequence MGQIVTMFEALPHIIDEVINIVIIVLIIITSIKAVYNFATCGILALVSFLFLAGRSCG (SEQ ID NO: 53). In some embodiments, a first chimeric polypeptide of a multi-chain chimeric polypeptide, a second chimeric polypeptide of the multi-chain chimeric polypeptide, or both includes a signal sequence having an amino acid sequence MPNHQSGSPTGSSDLLLSGKKQRPHLALRRKRRREMRKINRKVRRMNLAPIKEKTAWQ HLQALISEAEEVLKTSQTPQNSLTLFLALLSVLGPPVTG (SEQ ID NO: 54). In some embodiments, a first chimeric polypeptide of a multi-chain chimeric polypeptide, a second chimeric polypeptide of the multi-chain chimeric polypeptide, or both includes a signal sequence having an amino acid sequence MDSKGSSQKGSRLLLLLVVSNLLLCQGVVS (SEQ ID NO: 55). Those of ordinary skill in the art will be aware of other appropriate signal sequences for use in a first chimeric polypeptide and/or a second chimeric polypeptide of multi-chain chimeric polypeptides described herein.

In some embodiments, a first chimeric polypeptide of a multi-chain chimeric polypeptide, a second chimeric polypeptide of the multi-chain chimeric polypeptide, or both includes a signal sequence that is about 10 to 100 amino acids in length. For example, a signal sequence can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a signal sequence is about 10, 15, 20, 25, 30, 35, 40, 45, 50, 55, 60, 65, 70, 75, 80, 85, 90, 95, or 100 amino acids in length.

In some embodiments, any of the signal sequences disclosed herein can include one or more additional amino acids (e.g., 1, 2, 3, 5, 6, 7, 8, 9, 10, or more amino acids) at its N-terminus and/or C-terminus, so long as the function of the signal sequence remains intact. For example, a signal sequence having the amino acid sequence MKCLLYLAFLFLGVNC (SEQ ID NO: 56) can include one or more additional amino acids at the N-terminus or C-terminus, while still retaining the ability to direct a first chimeric polypeptide of a multi-chain chimeric polypeptide, a second chimeric polypeptide of the multi-chain chimeric polypeptide, or both to the secretory pathway.

In some embodiments, a first chimeric polypeptide of a multi-chain chimeric polypeptide, a second chimeric polypeptide of the multi-chain chimeric polypeptide, or both includes a signal sequence that directs the multi-chain chimeric polypeptide into the extracellular space. Such embodiments are useful in producing multi-chain chimeric polypeptides that are relatively easy to be isolated and/or purified.

### Exemplary First and Second Chimeric Polypeptides

In some embodiments of any of the multi-chain chimeric polypeptides described herein, the first target-binding domain and the second targeting-binding domain each independently bind specifically to TGF-β. In some examples of these multi-chain chimeric polypeptides, the first target-binding domain and the soluble tissue factor domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble tissue factor domain in the first chimeric polypeptide.

In some embodiments of these multi-chain chimeric polypeptides, the soluble tissue factor domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble tissue factor domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble tissue factor domain can be any of the exemplary soluble tissue factor domains described herein. In some embodiments of these multi-chain chimeric polypeptides, the pair of affinity domains can be any of the exemplary pairs of affinity domains described herein.

In some embodiments of these multi-chain chimeric polypeptides, the first target-binding domain and the second target-binding domain each independently bind specifically to TGF-β. In some embodiments of these multi-chain chimeric polypeptides, the first target-binding domain and the second target-binding domain bind specifically to the same epitope. In some embodiments of these multi-chain chimeric polypeptides, the first target-binding domain and the second target-binding domain include the same amino acid sequence.

In some embodiments of these multi-chain chimeric polypeptides, the first target-binding domain and the second target-binding domain is a soluble TGF-β receptor (e.g., a soluble TGFβRII receptor, e.g., a soluble human TGFβRII). In some embodiments of these multi-chain chimeric polypeptides, the soluble human TGFRβRII includes a first sequence of soluble human TGFRβRII and a second sequence of soluble human TGFRβRII. In some embodiments of these multi-chain chimeric polypeptides, the soluble human TGFRβRII includes a linker disposed between the first sequence of soluble human TGFRβRII and the second sequence of soluble human TGFRβRII. In some examples of these multi-chain chimeric polypeptides, the linker includes the sequence GGGGSGGGGSGGGGS (SEQ ID NO: 5).

In some embodiments of these multi-chain chimeric polypeptides, the first sequence of soluble human TGFRβRII receptor comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 2)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD.

In some embodiments of these multi-chain chimeric polypeptides, the second sequence of soluble human TGFRβRII receptor comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 2)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD.

In some embodiments of these multi-chain chimeric polypeptides, the first sequence of soluble human TGFRβRII receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 3)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGAT.

In some embodiments of these multi-chain chimeric polypeptides, the second sequence of soluble human TGFRβRII receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 4)
ATTCCTCCCCACGTGCAGAAGAGCGTGAATAATGACATGATCGTGACCG
ATAACAATGGCGCCGTGAAATTTCCCCAGCTGTGCAAATTCTGCGATGT
GAGGTTTTCCACCTGCGACAACCAGAAGTCCTGTATGAGCAACTGCTCC
ATCACCTCCATCTGTGAGAAGCCTCAGGAGGTGTGCGTGGCTGTCTGGC
GGAAGAATGACGAGAATATCACCCTGGAAACCGTCTGCCACGATCCCAA
GCTGCCCTACCACGATTTCATCCTGGAAGACGCCGCCAGCCCTAAGTGC
ATCATGAAAGAGAAAAAGAAGCCTGGCGAGACCTTTTTCATGTGCTCCT
GCAGCAGCGACGAATGCAACGACAATATCATCTTTAGCGAGGAATACAA
TACCAGCAACCCCGAC.

In some embodiments of these multi-chain chimeric polypeptides, the soluble TGF-β receptor includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 6)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD.

In some embodiments of these multi-chain chimeric polypeptides, the soluble TGF-β receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 7)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCTCCATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGAC.

In some embodiments, the first chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 8)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPDSGTTNTV
AAYNLTWKSTNFKTILEWEPKPVNQVYTVQISTKSGDWKSKCFYTTDTE
CDLTDEIVKDVKQTYLARVFSYPAGNVESTGSAGEPLYENSPEFTPYLE
TNLGQPTIQSFEQVGTKVNVTVEDERTLVRRNNTFLSLRDVFGKDLIYT
LYYWKSSSSGKKTAKTNTNEFLIDVDKGENYCFSVQAVIPSRTVNRKST
DSPVECMGQEKGEFRENWVNVISDLKKIEDLIQSMHIDATLYTESDVHP
SCKVTAMKCFLLELQVISLESGDASIHDTVENLIILANNSLSSNGNVTE
SGCKECEELEEKNIKEFLQSFVHIVQMFINTS.

In some embodiments, a first chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical at least 98% identical at least 99% identical or 100% identical to:

(SEQ ID NO: 9)
ATCCCCCCCCATGTGCAAAAGAGCGTGAACAACGATATGATCGTGACCGACAACAA
CGGCGCCGTGAAGTTTCCCCAGCTCTGCAAGTTCTGCGATGTCAGGTTCAGCACCTG
CGATAATCAGAAGTCCTGCATGTCCAACTGCAGCATCACCTCCATCTGCGAGAAGCC
CCAAGAAGTGTGCGTGGCCGTGTGGCGGAAAAATGACGAGAACATCACCCTGGAGA
CCGTGTGTCACGACCCCAAGCTCCCTTATCACGACTTCATTCTGGAGGACGCTGCCT
CCCCCAAATGCATCATGAAGGAGAAGAAGAAGCCCGGAGAGACCTTCTTTATGTGT
TCCTGTAGCAGCGACGAGTGTAACGACAACATCATCTTCAGCGAAGAGTACAACAC
CAGCAACCCTGATGGAGGTGGCGGATCCGGAGGTGGAGGTTCTGGTGGAGGTGGGA
GTATTCCTCCCCACGTGCAGAAGAGCGTGAATAATGACATGATCGTGACCGATAAC
AATGGCGCCGTGAAATTTCCCCAGCTGTGCAAATTCTGCGATGTGAGGTTTTCCACC
TGCGACAACCAGAAGTCCTGTATGAGCAACTGCTCCATCACCTCCATCTGTGAGAAG
CCTCAGGAGGTGTGCGTGGCTGTCTGGCGGAAGAATGACGAGAATATCACCCTGGA
AACCGTCTGCCACGATCCCAAGCTGCCCTACCACGATTTCATCCTGGAAGACGCCGC
CAGCCCTAAGTGCATCATGAAAGAGAAAAAGAAGCCTGGCGAGACCTTTTTCATGT
GCTCCTGCAGCAGCGACGAATGCAACGACAATATCATCTTTAGCGAGGAATACAAT
ACCAGCAACCCCGACAGCGGCACAACCAACACAGTCGCTGCCTATAACCTCACTTG
GAAGAGCACCAACTTCAAAACCATCCTCGAATGGGAACCCAAACCCGTTAACCAAG
TTTACACCGTGCAGATCAGCACCAAGTCCGGCGACTGGAAGTCCAAATGTTTCTATA
CCACCGACACCGAGTGCGATCTCACCGATGAGATCGTGAAAGATGTGAAACAGACC
TACCTCGCCCGGGTGTTTAGCTACCCCGCCGGCAATGTGGAGAGCACTGGTTCCGCT
GGCGAGCCTTTATACGAGAACAGCCCCGAATTTACCCCTTACCTCGAGACCAATTTA
GGACAGCCCACCATCCAAAGCTTTGAGCAAGTTGGCACAAAGGTGAATGTGACAGT
GGAGGACGAGCGGACTTTAGTGCGGCGGAACAACACCTTTCTCAGCCTCCGGGATG
TGTTCGGCAAAGATTTAATCTACACACTGTATTACTGGAAGTCCTCTTCCTCCGGCA
AGAAGACAGCTAAAACCAACACAAACGAGTTTTTAATCGACGTGGATAAAGGCGAA
AACTACTGTTTCAGCGTGCAAGCTGTGATCCCCTCCCGGACCGTGAATAGGAAAAGC
ACCGATAGCCCCGTTGAGTGCATGGGCCAAGAAAAGGGCGAGTTCCGGGAGAACTG
GGTGAACGTCATCAGCGATTTAAAGAAGATCGAAGATTTAATTCAGTCCATGCATAT
CGACGCCACTTTATACACAGAATCCGACGTGCACCCCTCTTGTAAGGTGACCGCCAT
GAAATGTTTTTTACTGGAGCTGCAAGTTATCTCTTTAGAGAGCGGAGACGCTAGCAT
CCACGACACCGTGGAGAATTTAATCATTTTAGCCAATAACTCTTTATCCAGCAACGG
CAACGTGACAGAGTCCGGCTGCAAGGAGTGCGAAGAGCTGGAGGAGAAGAACATC
AAGGAGTTTCTGCAATCCTTTGTGCACATTGTCCAGATGTTCATCAATACCTCC.

In some embodiments, a first chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 10)
MKWVTFISLLFLFSSAYSIPPHVQKSVNNDMIVTDNNGAVKFPQLCKFCDVRFSTCDNQ
KSCMSNCSITSICEKPQEVCVAVWRKNDENITLETVCHDPKLPYHDFILEDAASPKCIMK
EKKKPGETFFMCSCSSDECNDNIIFSEEYNTSNPDGGGGSGGGGSGGGGSIPPHVQKSVN
NDMIVTDNNGAVKFPQLCKFCDVRFSTCDNQKSCMSNCSITSICEKPQEVCVAVWRKN
DENITLETVCHDPKLPYHDFILEDAASPKCIMKEKKKPGETFFMCSCSSDECNDNIIFSEE
YNTSNPDSGTTNTVAAYNLTWKSTNFKTILEWEPKPVNQVYTVQISTKSGDWKSKCFY
TTDTECDLTDEIVKDVKQTYLARVFSYPAGNVESTGSAGEPLYENSPEFTPYLETNLGQP
TIQSFEQVGTKVNVTVEDERTLVRRNNTFLSLRDVFGKDLIYTLYYWKSSSSGKKTAKT
NTNEFLIDVDKGENYCFSVQAVIPSRTVNRKSTDSPVECMGQEKGEFRENWVNVISDLK
KIEDLIQSMHIDATLYTESDVHPSCKVTAMKCFLLELQVISLESGDASIHDTVENLIILAN
NSLSSNGNVTESGCKECEELEEKNIKEFLQSFVHIVQMFINTS.

In some embodiments, a first chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 11)
ATGAAGTGGGTGACCTTCATCAGCCTGCTGTTCCTGTTCTCCAGCGCCTACTCCATCC
CCCCCCATGTGCAAAAGAGCGTGAACAACGATATGATCGTGACCGACAACAACGGC
GCCGTGAAGTTTCCCCAGCTCTGCAAGTTCTGCGATGTCAGGTTCAGCACCTGCGAT
AATCAGAAGTCCTGCATGTCCAACTGCAGCATCACCTCCATCTGCGAGAAGCCCCAA
GAAGTGTGCGTGGCCGTGTGGCGGAAAAATGACGAGAACATCACCCTGGAGACCGT
GTGTCACGACCCCAAGCTCCCTTATCACGACTTCATTCTGGAGGACGCTGCCTCCCC
CAAATGCATCATGAAGGAGAAGAAGAAGCCCGGAGAGACCTTCTTTATGTGTTCCT
GTAGCAGCGACGAGTGTAACGACAACATCATCTTCAGCGAAGAGTACAACACCAGC
AACCCTGATGGAGGTGGCGGATCCGGAGGTGGAGGTTCTGGTGGAGGTGGGAGTAT
TCCTCCCCACGTGCAGAAGAGCGTGAATAATGACATGATCGTGACCGATAACAATG
GCGCCGTGAAATTTCCCCAGCTGTGCAAATTCTGCGATGTGAGGTTTTCCACCTGCG
ACAACCAGAAGTCCTGTATGAGCAACTGCTCCATCACCTCCATCTGTGAGAAGCCTC
AGGAGGTGTGCGTGGCTGTCTGGCGGAAGAATGACGAGAATATCACCCTGGAAACC
GTCTGCCACGATCCCAAGCTGCCCTACCACGATTTCATCCTGGAAGACGCCGCCAGC
CCTAAGTGCATCATGAAAGAGAAAAAGAAGCCTGGCGAGACCTTTTTCATGTGCTCC
TGCAGCAGCGACGAATGCAACGACAATATCATCTTTAGCGAGGAATACAATACCAG
CAACCCCGACAGCGGCACAACCAACACAGTCGCTGCCTATAACCTCACTTGGAAGA
GCACCAACTTCAAAACCATCCTCGAATGGGAACCCAAACCCGTTAACCAAGTTTAC
ACCGTGCAGATCAGCACCAAGTCCGGCGACTGGAAGTCCAAATGTTTCTATACCACC
GACACCGAGTGCGATCTCACCGATGAGATCGTGAAAGATGTGAAACAGACCTACCT
CGCCCGGGTGTTTAGCTACCCCGCCGGCAATGTGGAGAGCACTGGTTCCGCTGGCGA
GCCTTTATACGAGAACAGCCCCGAATTTACCCCTTACCTCGAGACCAATTTAGGACA
GCCCACCATCCAAAGCTTTGAGCAAGTTGGCACAAAGGTGAATGTGACAGTGGAGG
ACGAGCGGACTTTAGTGCGGCGGAACAACACCTTTCTCAGCCTCCGGGATGTGTTCG
GCAAAGATTTAATCTACACACTGTATTACTGGAAGTCCTCTTCCTCCGGCAAGAAGA
CAGCTAAAACCAACACAAACGAGTTTTTAATCGACGTGGATAAAGGCGAAAACTAC
TGTTTCAGCGTGCAAGCTGTGATCCCCTCCCGGACCGTGAATAGGAAAAGCACCGAT
AGCCCCGTTGAGTGCATGGGCCAAGAAAAGGGCGAGTTCCGGGAGAACTGGGTGAA
CGTCATCAGCGATTTAAAGAAGATCGAAGATTTAATTCAGTCCATGCATATCGACGC
CACTTTATACACAGAATCCGACGTGCACCCCTCTTGTAAGGTGACCGCCATGAAATG
TTTTTTACTGGAGCTGCAAGTTATCTCTTTAGAGAGCGGAGACGCTAGCATCCACGA
CACCGTGGAGAATTTAATCATTTTAGCCAATAACTCTTTATCCAGCAACGGCAACGT
GACAGAGTCCGGCTGCAAGGAGTGCGAAGAGCTGGAGGAGAAGAACATCAAGGAG
TTTCTGCAATCCTTTGTGCACATTGTCCAGATGTTCATCAATACCTCC.

In some embodiments, the second chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 16)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPDITCPPPM
SVEHADIWVKSYSLYSRERYICNSGFKRKAGTSSLTECVLNKATNVAHW
TTPSLKCIR.

In some embodiments, a second chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 17)
ATCCCCCCCCATGTGCAAAAGAGCGTGAACAACGATATGATCGTGACCGACAACAA
CGGCGCCGTGAAGTTTCCCCAGCTCTGCAAGTTCTGCGATGTCAGGTTCAGCACCTG
CGATAATCAGAAGTCCTGCATGTCCAACTGCAGCATCACCTCCATCTGCGAGAAGCC
CCAAGAAGTGTGCGTGGCCGTGTGGCGGAAAAATGACGAGAACATCACCCTGGAGA
CCGTGTGTCACGACCCCAAGCTCCCTTATCACGACTTCATTCTGGAGGACGCTGCCT
CCCCCAAATGCATCATGAAGGAGAAGAAGAAGCCCGGAGAGACCTTCTTTATGTGT
TCCTGTAGCAGCGACGAGTGTAACGACAACATCATCTTCAGCGAAGAGTACAACAC
CAGCAACCCTGATGGAGGTGGCGGATCCGGAGGTGGAGGTTCTGGTGGAGGTGGGA
GTATTCCTCCCCACGTGCAGAAGAGCGTGAATAATGACATGATCGTGACCGATAAC
AATGGCGCCGTGAAATTTCCCCAGCTGTGCAAATTCTGCGATGTGAGGTTTTCCACC
TGCGACAACCAGAAGTCCTGTATGAGCAACTGCTCCATCACCTCCATCTGTGAGAAG
CCTCAGGAGGTGTGCGTGGCTGTCTGGCGGAAGAATGACGAGAATATCACCCTGGA
AACCGTCTGCCACGATCCCAAGCTGCCCTACCACGATTTCATCCTGGAAGACGCCGC
CAGCCCTAAGTGCATCATGAAAGAGAAAAAGAAGCCTGGCGAGACCTTTTTCATGT
GCTCCTGCAGCAGCGACGAATGCAACGACAATATCATCTTTAGCGAGGAATACAAT
ACCAGCAACCCCGACATTACATGCCCCCCTCCCATGAGCGTGGAGCACGCCGACAT
CTGGGTGAAGAGCTATAGCCTCTACAGCCGGGAGAGGTATATCTGTAACAGCGGCT
TCAAGAGGAAGGCCGGCACCAGCAGCCTCACCGAGTGCGTGCTGAATAAGGCTACC
AACGTGGCTCACTGGACAACACCCTCTTTAAAGTGCATCCGG.

In some embodiments, a second chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical at least 98% identical, at least 99% identical, or 100% identical) to

(SEQ ID NO: 18)
MKWVTFISLLFLFSSAYSIPPHVQKSVNNDMIVTDNNGAVKFPQLCKFCDVRFSTCDNQ
KSCMSNCSITSICEKPQEVCVAVWRKNDENITLETVCHDPKLPYHDFILEDAASPKCIMK
EKKKPGETFFMCSCSSDECNDNIIFSEEYNTSNPDGGGGSGGGGSGGGGSIPPHVQKSVN
NDMIVTDNNGAVKFPQLCKFCDVRFSTCDNQKSCMSNCSITSICEKPQEVCVAVWRKN
DENITLETVCHDPKLPYHDFILEDAASPKCIMKEKKKPGETFFMCSCSSDECNDNIIFSEE
YNTSNPDITCPPPMSVEHADIWVKSYSLYSRERYICNSGFKRKAGTSSLTECVLNKATNV
AHWTTPSLKCIR.

In some embodiments, a second chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 19)
ATGAAGTGGGTGACCTTCATCAGCCTGCTGTTCCTGTTCTCCAGCGCCTACTCCATCC
CCCCCCATGTGCAAAAGAGCGTGAACAACGATATGATCGTGACCGACAACAACGGC
GCCGTGAAGTTTCCCCAGCTCTGCAAGTTCTGCGATGTCAGGTTCAGCACCTGCGAT
AATCAGAAGTCCTGCATGTCCAACTGCAGCATCACCTCCATCTGCGAGAAGCCCCAA
GAAGTGTGCGTGGCCGTGTGGCGGAAAAATGACGAGAACATCACCCTGGAGACCGT
GTGTCACGACCCCAAGCTCCCTTATCACGACTTCATTCTGGAGGACGCTGCCTCCCC
CAAATGCATCATGAAGGAGAAGAAGAAGCCCGGAGAGACCTTCTTTATGTGTTCCT
GTAGCAGCGACGAGTGTAACGACAACATCATCTTCAGCGAAGAGTACAACACCAGC
AACCCTGATGGAGGTGGCGGATCCGGAGGTGGAGGTTCTGGTGGAGGTGGGAGTAT
TCCTCCCCACGTGCAGAAGAGCGTGAATAATGACATGATCGTGACCGATAACAATG
GCGCCGTGAAATTTCCCCAGCTGTGCAAATTCTGCGATGTGAGGTTTTCCACCTGCG
ACAACCAGAAGTCCTGTATGAGCAACTGCTCCATCACCTCCATCTGTGAGAAGCCTC
AGGAGGTGTGCGTGGCTGTCTGGCGGAAGAATGACGAGAATATCACCCTGGAAACC
GTCTGCCACGATCCCAAGCTGCCCTACCACGATTTCATCCTGGAAGACGCCGCCAGC
CCTAAGTGCATCATGAAAGAGAAAAAGAAGCCTGGCGAGACCTTTTTCATGTGCTCC
TGCAGCAGCGACGAATGCAACGACAATATCATCTTTAGCGAGGAATACAATACCAG
CAACCCCGACATTACATGCCCCCCTCCCATGAGCGTGGAGCACGCCGACATCTGGGT
GAAGAGCTATAGCCTCTACAGCCGGGAGAGGTATATCTGTAACAGCGGCTTCAAGA
GGAAGGCCGGCACCAGCAGCCTCACCGAGTGCGTGCTGAATAAGGCTACCAACGTG
GCTCACTGGACAACACCCTCTTTAAAGTGCATCCGG.

In some embodiments, a first chimeric polypeptide can include a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical or 100% identical to:

(SEQ ID NO: 12)
IPPHVQKSVNNDMIVTDNNGAVKFPQLCKFCDVRFSTCDNQKSCMSNCSITSICEKPQEV
CVAVWRKNDENITLETVCHDPKLPYHDFILEDAASPKCIMKEKKKPGETFFMCSCSSDE
CNDNIIFSEEYNTSNPDGGGGSGGGGSGGGGSIPPHVQKSVNNDMIVTDNNGAVKFPQL
CKFCDVRFSTCDNQKSCMSNCSITSICEKPQEVCVAVWRKNDENITLETVCHDPKLPYH
DFILEDAASPKCIMKEKKKPGETFFMCSCSSDECNDNIIFSEEYNTSNPDSGTTNTVAAYN
LTWKSTNFKTILEWEPKPVNQVYTVQISTKSGDWKSKCFYTTDTECDLTDEIVKDVKQT
YLARVFSYPAGNVESTGSAGEPLYENSPEFTPYLETNLGQPTIQSFEQVGTKVNVTVEDE
RTLVRRNNTFLSLRDVFGKDLIYTLYYWKSSSSGKKTAKTNTNEFLIDVDKGENYCFSV
QAVIPSRTVNRKSTDSPVECMGQEKGEFRENWVNVISNLKKIEDLIQSMHIDATLYTESD
VHPSCKVTAMKCFLLELQVISLESGDASIHDTVENLIILANNSLSSNGNVTESGCKECEEL
EEKNIKEFLQSFVHIVQMFINTS.

In some embodiments, a first chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 13)
ATCCCACCGCACGTTCAGAAGTCGGTGAATAACGACATGATAGTCACTGACAACAA
CGGTGCAGTCAAGTTTCCACAACTGTGTAAATTTTGTGATGTGAGATTTTCCACCTGT
GACAACCAGAAATCCTGCATGAGCAACTGCAGCATCACCTCCATCTGTGAGAAGCC
ACAGGAAGTCTGTGTGGCTGTATGGAGAAAGAATGACGAGAACATAACACTAGAGA
CAGTTTGCCATGACCCCAAGCTCCCCTACCATGACTTTATTCTGGAAGATGCTGCTTC
TCCAAAGTGCATTATGAAGGAAAAAAAAAAGCCTGGTGAGACTTTCTTCATGTGTTC
CTGTAGCTCTGATGAGTGCAATGACAACATCATCTTCTCAGAAGAATATAACACCAG
CAATCCTGACGGAGGTGGCGGATCCGGAGGTGGAGGTTCTGGTGGAGGTGGGAGTA
TTCCTCCCCACGTGCAGAAGAGCGTGAATAATGACATGATCGTGACCGATAACAAT
GGCGCCGTGAAATTTCCCCAGCTGTGCAAATTCTGCGATGTGAGGTTTTCCACCTGC
GACAACCAGAAGTCCTGTATGAGCAACTGCTCCATCACCTCCATCTGTGAGAAGCCT
CAGGAGGTGTGCGTGGCTGTCTGGCGGAAGAATGACGAGAATATCACCCTGGAAAC
CGTCTGCCACGATCCCAAGCTGCCCTACCACGATTTCATCCTGGAAGACGCGCCAG
CCCTAAGTGCATCATGAAAGAGAAAAAGAAGCCTGGCGAGACCTTTTTCATGTGCTC
CTGCAGCAGCGACGAATGCAACGACAATATCATCTTTAGCGAGGAATACAATACCA
GCAACCCCGACTCAGGCACTACAAATACTGTGGCAGCATATAATTTAACTTGGAAAT
CAACTAATTTCAAGACAATTTTGGAGTGGGAACCCAAACCCGTCAATCAAGTCTACA
CTGTTCAAATAAGCACTAAGTCAGGAGATTGGAAAAGCAAATGCTTTTACACAACA
GACACAGAGTGTGACCTCACCGACGAGATTGTGAAGGATGTGAAGCAGACGTACTT
GGCACGGGTCTTCTCCTACCCGGCAGGGAATGTGGAGAGCACCGGTTCTGCTGGGG
AGCCTCTGTATGAGAACTCCCCAGAGTTCACACCTTACCTGGAGACAAACCTCGGAC
AGCCAACAATTCAGAGTTTTGAACAGGTGGGAACAAAAGTGAATGTGACCGTAGAA
GATGAACGGACTTTAGTCAGAAGGAACAACACTTTCCTAAGCCTCCGGGATGTTTTT
GGCAAGGACTTAATTTATACACTTTATTATTGGAAATCTTCAAGTTCAGGAAAGAAA
ACAGCCAAAACAAACACTAATGAGTTTTTGATTGATGTGGATAAAGGAGAAAACTA
CTGTTTCAGTGTTCAAGCAGTGATTCCCTCCCGAACAGTTAACCGGAAGAGTACAGA
CAGCCCGGTAGAGTGTATGGGCCAGGAGAAAGGGGAATTCAGAGAAAACTGGGTG
AATGTAATAAGTAATTTGAAAAAAATTGAAGATCTTATTCAATCTATGCATATTGAT
GCTACTTTATATACGGAAAGTGATGTTCACCCCAGTTGCAAAGTAACAGCAATGAAG
TGCTTTCTCTTGGAGTTACAAGTTATTTCACTTGAGTCCGGAGATGCAAGTATTCATG
ATACAGTAGAAAATCTGATCATCCTAGCAAACAACAGTTTGTCTTCTAATGGGAATG
TAACAGAATCTGGATGCAAAGAATGTGAGGAACTGGAGGAAAAAAATATTAAAGA
ATTTTTGCAGAGTTTTGTACATATTGTCCAAATGTTCATCAACACTTCT.

In some embodiments, a first chimeric polypeptide can include a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 14)
MGVKVLFALICIAVAEAIPPHVQKSVNNDMIVTDNNGAVKFPQLCKFCDVRFSTCDNQK
SCMSNCSITSICEKPQEVCVAVWRKNDENITLETVCHDPKLPYHDFILEDAASPKCIMKE
KKKPGETFFMCSCSSDECNDNIIFSEEYNTSNPDGGGGSGGGGSGGGGSIPPHVQKSVNN
DMIVTDNNGAVKFPQLCKFCDVRFSTCDNQKSCMSNCSITSICEKPQEVCVAVWRKND
ENITLETVCHDPKLPYHDFILEDAASPKCIMKEKKKPGETFFMCSCSSDECNDNIIFSEEY
NTSNPDSGTTNTVAAYNLTWKSTNFKTILEWEPKPVNQVYTVQISTKSGDWKSKCFYTT
DTECDLTDEIVKDVKQTYLARVFSYPAGNVESTGSAGEPLYENSPEFTPYLETNLGQPTI
QSFEQVGTKVNVTVEDERTLVRRNNTFLSLRDVFGKDLIYTLYYWKSSSSGKKTAKTNT
NEFLIDVDKGENYCFSVQAVIPSRTVNRKSTDSPVECMGQEKGEFRENWVNVISNLKKI
EDLIQSMHIDATLYTESDVHPSCKVTAMKCFLLELQVISLESGDASIHDTVENLIILANNS
LSSNGNVTESGCKECEELEEKNIKEFLQSFVHIVQMFINTS.

In some embodiments, a first chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 15)
ATGGGAGTGAAAGTTCTTTTTGCCCTTATTTGTATTGCTGTGGCCGAGGCC
ATCCCACCGCACGTTCAGAAGTCGGTGAATAACGACATGATAGTCACTGACAACAA
CGGTGCAGTCAAGTTTCCACAACTGTGTAAATTTTGTGATGTGAGATTTTCCACCTGT
GACAACCAGAAATCCTGCATGAGCAACTGCAGCATCACCTCCATCTGTGAGAAGCC
ACAGGAAGTCTGTGTGGCTGTATGGAGAAAGAATGACGAGAACATAACACTAGAGA
CAGTTTGCCATGACCCCAAGCTCCCCTACCATGACTTTATTCTGGAAGATGCTGCTTC
TCCAAAGTGCATTATGAAGGAAAAAAAAAAGCCTGGTGAGACTTTCTTCATGTGTTC
CTGTAGCTCTGATGAGTGCAATGACAACATCATCTTCTCAGAAGAATATAACACCAG
CAATCCTGACGGAGGTGGCGGATCCGGAGGTGGAGGTTCTGGTGGAGGTGGGAGTA
TTCCTCCCCACGTGCAGAAGAGCGTGAATAATGACATGATCGTGACCGATAACAAT
GGCGCCGTGAAATTTCCCCAGCTGTGCAAATTCTGCGATGTGAGGTTTTCCACCTGC
GACAACCAGAAGTCCTGTATGAGCAACTGCTCCATCACCTCCATCTGTGAGAAGCCT
CAGGAGGTGTGCGTGGCTGTCTGGCGGAAGAATGACGAGAATATCACCCTGGAAAC
CGTCTGCCACGATCCCAAGCTGCCCTACCACGATTTCATCCTGGAAGACGCCGCCAG
CCCTAAGTGCATCATGAAAGAGAAAAAGAAGCCTGGCGAGACCTTTTTCATGTGCTC
CTGCAGCAGCGACGAATGCAACGACAATATCATCTTTAGCGAGGAATACAATACCA
GCAACCCCGACTCAGGCACTACAAATACTGTGGCAGCATATAATTTAACTTGGAAAT
CAACTAATTTCAAGACAATTTTGGAGTGGGAACCCAAACCCGTCAATCAAGTCTACA
CTGTTCAAATAAGCACTAAGTCAGGAGATTGGAAAAGCAAATGCTTTTACACAACA
GACACAGAGTGTGACCTCACCGACGAGATTGTGAAGGATGTGAAGCAGACGTACTT
GGCACGGGTCTTCTCCTACCCGGCAGGGAATGTGGAGAGCACCGGTTCTGCTGGGG
AGCCTCTGTATGAGAACTCCCCAGAGTTCACACCTTACCTGGAGACAAACCTCGGAC
AGCCAACAATTCAGAGTTTTGAACAGGTGGGAACAAAAGTGAATGTGACCGTAGAA
GATGAACGGACTTTAGTCAGAAGGAACAACACTTTCCTAAGCCTCCGGGATGTTTTT
GGCAAGGACTTAATTTATACACTTTATTATTGGAAATCTTCAAGTTCAGGAAAGAAA
ACAGCCAAAACAAACACTAATGAGTTTTTGATTGATGTGGATAAAGGAGAAAACTA
CTGTTTCAGTGTTCAAGCAGTGATTCCCTCCCGAACAGTTAACCGGAAGAGTACAGA
CAGCCCGGTAGAGTGTATGGGCCAGGAGAAAGGGGAATTCAGAGAAAACTGGGTG
AATGTAATAAGTAATTTGAAAAAAATTGAAGATCTTATTCAATCTATGCATATTGAT
GCTACTTTATATACGGAAAGTGATGTTCACCCCAGTTGCAAAGTAACAGCAATGAAG
TGCTTTCTCTTGGAGTTACAAGTTATTTCACTTGAGTCCGGAGATGCAAGTATTCATG
ATACAGTAGAAAATCTGATCATCCTAGCAAACAACAGTTTGTCTTCTAATGGGAATG
TAACAGAATCTGGATGCAAAGAATGTGAGGAACTGGAGGAAAAAAATATTAAAGA
ATTTTTGCAGAGTTTTGTACATATTGTCCAAATGTTCATCAACACTTCT.

### Exemplary First and Second Chimeric Polypeptides

Exemplary multi-chain chimeric polypeptides provided herein include: (a) a first chimeric polypeptide comprising: (i) a first target-binding domain comprising: a first sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; and a second sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; (ii) a soluble tissue factor domain; and (iii) a first domain of a pair of affinity domains; (b) a second chimeric polypeptide comprising: (i) a second domain of a pair of affinity domains; and (ii) a second target-binding domain comprising: a first sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; and a second sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

In some embodiments, the first sequence of the first target-binding domain comprises an asparagine at amino acid position 32 in SEQ ID NO: 20. In some embodiments, the first sequence of the first target-binding domain comprises an alanine at amino acid position 119 in SEQ ID NO: 20. In some embodiments, the first sequence of the first target-binding domain comprises an asparagine at amino acid position 32 and an alanine at amino acid position 119 in SEQ ID NO: 20.

In some embodiments, the second sequence of the first target-binding domain comprises an asparagine at amino acid position 32 in SEQ ID NO: 20. In some embodiments, the second sequence of the first target-binding domain comprises an alanine at amino acid position 119 in SEQ ID NO: 20. In some embodiments, the second sequence of the first target-binding domain comprises an asparagine at amino acid position 32 and an alanine at amino acid position 119 in SEQ ID NO: 20.

In some embodiments, the first sequence of the second target-binding domain comprises an asparagine at amino acid position 32 in SEQ ID NO: 20. In some embodiments, the first sequence of the second target-binding domain comprises an alanine at amino acid position 119 in SEQ ID NO: 20. In some embodiments, the first sequence of the second target-binding domain comprises an asparagine at amino acid position 32 and an alanine at amino acid position 119 in SEQ ID NO: 20.

In some embodiments, the second sequence of the second target-binding domain comprises an asparagine at amino acid position 32 in SEQ ID NO: 20. In some embodiments, the second sequence of the second target-binding domain comprises an alanine at amino acid position 119 in SEQ ID NO: 20. In some embodiments, the second sequence of the second target-binding domain comprises an asparagine at amino acid position 32 and an alanine at amino acid position 119 in SEQ ID NO: 20.

In some embodiments, the first target-binding domain and the soluble tissue factor domain directly abut each other in the first chimeric polypeptide. In some embodiments, the first chimeric polypeptide further comprises a linker sequence between the first target-binding domain and the soluble tissue factor domain in the first chimeric polypeptide.

In some embodiments, the soluble tissue factor domain and the first domain of the pair of affinity domains directly abut each other in the first chimeric polypeptide. In some embodiments, the first chimeric polypeptide further comprises a linker sequence between the soluble tissue factor domain and the first domain of the pair of affinity domains in the first chimeric polypeptide.

In some embodiments, the second domain of the pair of affinity domains and the second target-binding domain directly abut each other in the second chimeric polypeptide. In some embodiments, the second chimeric polypeptide further comprises a linker sequence between the second domain of the pair of affinity domains and the second target-binding domain in the second chimeric polypeptide.

In some embodiments, the first chimeric polypeptide comprises a sequence that is at least 80% identical, at least 82% identical, at least 84% identical, at least 85%, identical, at least 86% identical, at least 88% identical, at least 90% identical, at least 92% identical, at least 94% identical, at least 95% identical, at least 96% identical, at least 98% identical, at least 99% identical, or 100% identical to SEQ ID NO: 28 or 30.

In some embodiments, the second chimeric polypeptide comprises a sequence that is at least 80% identical, at least 82% identical, at least 84% identical, at least 85%, identical, at least 86% identical, at least 88% identical, at least 90% identical, at least 92% identical, at least 94% identical, at least 95% identical, at least 96% identical, at least 98% identical, at least 99% identical, or 100% identical to SEQ ID NO: 32 or 34.

Exemplary First Chimeric Polypeptide
(SEQ ID NO: 28)
IPPHVQKSVNNDMIVTDNNGAVKFPQLCKFCNVRFSTCDNQKSCMSNCSITSICEKPQEV
CVAVWRKNDENITLETVCHDPKLPYHDFILEDAASPKCIMKEKKKPGETFFMCSCSSDA
CNDNIIFSEEYNTSNPDGGGGSGGGGSGGGGSIPPHVQKSVNNDMIVTDNNGAVKFPQL
CKFCNVRFSTCDNQKSCMSNCSITSICEKPQEVCVAVWRKNDENITLETVCHDPKLPYH
DFILEDAASPKCIMKEKKKPGETFFMCSCSSDACNDNIIFSEEYNTSNPDSGTTNTVAAY
NLTWKSTNFKTILEWEPKPVNQVYTVQISTKSGDWKSKCFYTTDTECDLTDEIVKDVKQ
TYLARVFSYPAGNVESTGSAGEPLYENSPEFTPYLETNLGQPTIQSFEQVGTKVNVTVED
ERTLVRRNNTFLSLRDVFGKDLIYTLYYWKSSSSGKKTAKTNTNEFLIDVDKGENYCFS
VQAVIPSRTVNRKSTDSPVECMGQEKGEFRENWVNVISDLKKIEDLIQSMHIDATLYTES
DVHPSCKVTAMKCFLLELQVISLESGDASIHDTVENLIILANNSLSSNGNVTESGCKECEE
LEEKNIKEFLQSFVHIVQMFINTS
Exemplary First Chimeric Polypeptide
(SEQ ID NO: 30)
MKWVTFISLLFLFSSAYSIPPHVQKSVNNDMIVTDNNGAVKFPQLCKFCNVRFSTCDNQ
KSCMSNCSITSICEKPQEVCVAVWRKNDENITLETVCHDPKLPYHDFILEDAASPKCIMK
EKKKPGETFFMCSCSSDACNDNIIFSEEYNTSNPDGGGGSGGGGSGGGGSIPPHVQKSVN
NDMIVTDNNGAVKFPQLCKFCNVRFSTCDNQKSCMSNCSITSICEKPQEVCVAVWRKN
DENITLETVCHDPKLPYHDFILEDAASPKCIMKEKKKPGETFFMCSCSSDACNDNIIFSEE
YNTSNPDSGTTNTVAAYNLTWKSTNFKTILEWEPKPVNQVYTVQISTKSGDWKSKCFY
TTDTECDLTDEIVKDVKQTYLARVFSYPAGNVESTGSAGEPLYENSPEFTPYLETNLGQP
TIQSFEQVGTKVNVTVEDERTLVRRNNTFLSLRDVFGKDLIYTLYYWKSSSSGKKTAKT
NTNEFLIDVDKGENYCFSVQAVIPSRTVNRKSTDSPVECMGQEKGEFRENWVNVISDLK
KIEDLIQSMHIDATLYTESDVHPSCKVTAMKCFLLELQVISLESGDASIHDTVENLIILAN
NSLSSNGNVTESGCKECEELEEKNIKEFLQSFVHIVQMFINTS
Exemplary Second Chimeric Polypeptide
(SEQ ID NO: 32)
IPPHVQKSVNNDMIVTDNNGAVKFPQLCKFCNVRFSTCDNQKSCMSNCSITSICEKPQEV
CVAVWRKNDENITLETVCHDPKLPYHDFILEDAASPKCIMKEKKKPGETFFMCSCSSDA
CNDNIIFSEEYNTSNPDGGGGSGGGGSGGGGSIPPHVQKSVNNDMIVTDNNGAVKFPQL
CKFCNVRFSTCDNQKSCMSNCSITSICEKPQEVCVAVWRKNDENITLETVCHDPKLPYH
DFILEDAASPKCIMKEKKKPGETFFMCSCSSDACNDNIIFSEEYNTSNPDITCPPPMSVEH
ADIWVKSYSLYSRERYICNSGFKRKAGTSSLTECVLNKATNVAHWTTPSLKCIR
Exemplary Second Chimeric Polypeptide
(SEQ ID NO: 34)
MKWVTFISLLFLFSSAYSIPPHVQKSVNNDMIVTDNNGAVKFPQLCKFCNVRFSTCDNQ
KSCMSNCSITSICEKPQEVCVAVWRKNDENITLETVCHDPKLPYHDFILEDAASPKCIMK
EKKKPGETFFMCSCSSDACNDNIIFSEEYNTSNPDGGGGSGGGGSGGGGSIPPHVQKSVN
NDMIVTDNNGAVKFPQLCKFCNVRFSTCDNQKSCMSNCSITSICEKPQEVCVAVWRKN
DENITLETVCHDPKLPYHDFILEDAASPKCIMKEKKKPGETFFMCSCSSDACNDNIIFSEE
YNTSNPDITCPPPMSVEHADIWVKSYSLYSRERYICNSGFKRKAGTSSLTECVLNKATNV
AHWTTPSLKCIR

In some embodiments, the first chimeric polypeptide is encoded by a nucleic acid comprising a sequence that is at least 80% identical, at least 82% identical, at least 84% identical, at least 85%, identical, at least 86% identical, at least 88% identical, at least 90% identical, at least 92% identical, at least 94% identical, at least 95% identical, at least 96% identical, at least 98% identical, at least 99% identical, or 100% identical to SEQ ID NO: 29 or 31.

In some embodiments, the second chimeric polypeptide is encoded by a nucleic acid comprising a sequence that is at least 80% identical, at least 82% identical, at least 84% identical, at least 85%, identical, at least 86% identical, at least 88% identical, at least 90% identical, at least 92% identical, at least 94% identical, at least 95% identical, at least 96% identical, at least 98% identical, at least 99% identical, or 100% identical to SEQ ID NO: 33 or 35.

Exemplary Nucleic Acid Encoding an Exemplary First Chimeric
Polypeptide
(SEQ ID NO: 29)
ATCCCCCCCCATGTGCAAAAGAGCGTGAACAACGATATGATCGTGACCGACAACAA
CGGCGCCGTGAAGTTTCCCCAGCTCTGCAAGTTCTGCAACGTCAGGTTCAGCACCTG
CGATAATCAGAAGTCCTGCATGTCCAACTGCAGCATCACCTCCATCTGCGAGAAGCC
CCAAGAAGTGTGCGTGGCCGTGTGGCGGAAAAATGACGAGAACATCACCCTGGAGA
CCGTGTGTCACGACCCCAAGCTCCCTTATCACGACTTCATTCTGGAGGACGCTGCCT
CCCCCAAATGCATCATGAAGGAGAAGAAGAAGCCCGGAGAGACCTTCTTTATGTGT
TCCTGTAGCAGCGACGCCTGTAACGACAACATCATCTTCAGCGAAGAGTACAACAC
CAGCAACCCTGATGGAGGTGGCGGATCCGGAGGTGGAGGTTCTGGTGGAGGTGGGA
GTATTCCTCCCCACGTGCAGAAGAGCGTGAATAATGACATGATCGTGACCGATAAC
AATGGCGCCGTGAAATTTCCCCAGCTGTGCAAATTCTGCAATGTGAGGTTTTCCACC
TGCGACAACCAGAAGTCCTGTATGAGCAACTGCTCCATCACCTCCATCTGTGAGAAG
CCTCAGGAGGTGTGCGTGGCTGTCTGGCGGAAGAATGACGAGAATATCACCCTGGA
AACCGTCTGCCACGATCCCAAGCTGCCCTACCACGATTTCATCCTGGAAGACGCCGC
CAGCCCTAAGTGCATCATGAAAGAGAAAAAGAAGCCTGGCGAGACCTTTTTCATGT
GCTCCTGCAGCAGCGACGCTTGCAACGACAATATCATCTTTAGCGAGGAATACAATA
CCAGCAACCCCGACAGCGGCACAACCAACACAGTCGCTGCCTATAACCTCACTTGG
AAGAGCACCAACTTCAAAACCATCCTCGAATGGGAACCCAAACCCGTTAACCAAGT
TTACACCGTGCAGATCAGCACCAAGTCCGGCGACTGGAAGTCCAAATGTTTCTATAC
CACCGACACCGAGTGCGATCTCACCGATGAGATCGTGAAAGATGTGAAACAGACCT
ACCTCGCCCGGGTGTTTAGCTACCCCGCCGGCAATGTGGAGAGCACTGGTTCCGCTG
GCGAGCCTTTATACGAGAACAGCCCCGAATTTACCCCTTACCTCGAGACCAATTTAG
GACAGCCCACCATCCAAAGCTTTGAGCAAGTTGGCACAAAGGTGAATGTGACAGTG
GAGGACGAGCGGACTTTAGTGCGGCGGAACAACACCTTTCTCAGCCTCCGGGATGT
GTTCGGCAAAGATTTAATCTACACACTGTATTACTGGAAGTCCTCTTCCTCCGGCAA
GAAGACAGCTAAAACCAACACAAACGAGTTTTTAATCGACGTGGATAAAGGCGAAA
ACTACTGTTTCAGCGTGCAAGCTGTGATCCCCTCCCGGACCGTGAATAGGAAAAGCA
CCGATAGCCCCGTTGAGTGCATGGGCCAAGAAAAGGGCGAGTTCCGGGAGAACTGG
GTGAACGTCATCAGCGATTTAAAGAAGATCGAAGATTTAATTCAGTCCATGCATATC
GACGCCACTTTATACACAGAATCCGACGTGCACCCCTCTTGTAAGGTGACCGCCATG
AAATGTTTTTTACTGGAGCTGCAAGTTATCTCTTTAGAGAGCGGAGACGCTAGCATC
CACGACACCGTGGAGAATTTAATCATTTTAGCCAATAACTCTTTATCCAGCAACGGC
AACGTGACAGAGTCCGGCTGCAAGGAGTGCGAAGAGCTGGAGGAGAAGAACATCA
AGGAGTTTCTGCAATCCTTTGTGCACATTGTCCAGATGTTCATCAATACCTCC
Exemplary Nucleic Acid Encoding an Exemplary First Chimeric
Polypeptide
(SEQ ID NO: 31)
ATGAAGTGGGTGACCTTCATCAGCCTGCTGTTCCTGTTCTCCAGCGCCTACTCCATCC
CCCCCCATGTGCAAAAGAGCGTGAACAACGATATGATCGTGACCGACAACAACGGC
GCCGTGAAGTTTCCCCAGCTCTGCAAGTTCTGCAACGTCAGGTTCAGCACCTGCGAT
AATCAGAAGTCCTGCATGTCCAACTGCAGCATCACCTCCATCTGCGAGAAGCCCCAA
GAAGTGTGCGTGGCCGTGTGGCGGAAAAATGACGAGAACATCACCCTGGAGACCGT
GTGTCACGACCCCAAGCTCCCTTATCACGACTTCATTCTGGAGGACGCTGCCTCCCC
CAAATGCATCATGAAGGAGAAGAAGAAGCCCGGAGAGACCTTCTTTATGTGTTCCT
GTAGCAGCGACGCCTGTAACGACAACATCATCTTCAGCGAAGAGTACAACACCAGC
AACCCTGATGGAGGTGGCGGATCCGGAGGTGGAGGTTCTGGTGGAGGTGGGAGTAT
TCCTCCCCACGTGCAGAAGAGCGTGAATAATGACATGATCGTGACCGATAACAATG
GCGCCGTGAAATTTCCCCAGCTGTGCAAATTCTGCAATGTGAGGTTTTCCACCTGCG
ACAACCAGAAGTCCTGTATGAGCAACTGCTCCATCACCTCCATCTGTGAGAAGCCTC
AGGAGGTGTGCGTGGCTGTCTGGCGGAAGAATGACGAGAATATCACCCTGGAAACC
GTCTGCCACGATCCCAAGCTGCCCTACCACGATTTCATCCTGGAAGACGCCGCCAGC
CCTAAGTGCATCATGAAAGAGAAAAAGAAGCCTGGCGAGACCTTTTTCATGTGCTCC
TGCAGCAGCGACGCTTGCAACGACAATATCATCTTTAGCGAGGAATACAATACCAG
CAACCCCGACAGCGGCACAACCAACACAGTCGCTGCCTATAACCTCACTTGGAAGA
GCACCAACTTCAAAACCATCCTCGAATGGGAACCCAAACCCGTTAACCAAGTTTAC
ACCGTGCAGATCAGCACCAAGTCCGGCGACTGGAAGTCCAAATGTTTCTATACCACC
GACACCGAGTGCGATCTCACCGATGAGATCGTGAAAGATGTGAAACAGACCTACCT
CGCCCGGGTGTTTAGCTACCCCGCCGGCAATGTGGAGAGCACTGGTTCCGCTGGCGA
GCCTTTATACGAGAACAGCCCCGAATTTACCCCTTACCTCGAGACCAATTTAGGACA
GCCCACCATCCAAAGCTTTGAGCAAGTTGGCACAAAGGTGAATGTGACAGTGGAGG
ACGAGCGGACTTTAGTGCGGCGGAACAACACCTTTCTCAGCCTCCGGGATGTGTTCG
GCAAAGATTTAATCTACACACTGTATTACTGGAAGTCCTCTTCCTCCGGCAAGAAGA
CAGCTAAAACCAACACAAACGAGTTTTTAATCGACGTGGATAAAGGCGAAAACTAC
TGTTTCAGCGTGCAAGCTGTGATCCCCTCCCGGACCGTGAATAGGAAAAGCACCGAT
AGCCCCGTTGAGTGCATGGGCCAAGAAAAGGGCGAGTTCCGGGAGAACTGGGTGAA
CGTCATCAGCGATTTAAAGAAGATCGAAGATTTAATTCAGTCCATGCATATCGACGC
CACTTTATACACAGAATCCGACGTGCACCCCTCTTGTAAGGTGACCGCCATGAAATG
TTTTTTACTGGAGCTGCAAGTTATCTCTTTAGAGAGCGGAGACGCTAGCATCCACGA
CACCGTGGAGAATTTAATCATTTTAGCCAATAACTCTTTATCCAGCAACGGCAACGT
GACAGAGTCCGGCTGCAAGGAGTGCGAAGAGCTGGAGGAGAAGAACATCAAGGAG
TTTCTGCAATCCTTTGTGCACATTGTCCAGATGTTCATCAATACCTCC
Exemplary Nucleic Acid Encoding an Exemplary Second Chimeric
Polypeptide
(SEQ ID NO: 33)
ATCCCACCTCACGTGCAGAAAAGCGTCAATAATGACATGATCGTGACTGACAATAA
CGGCGCCGTCAAGTTTCCACAGCTGTGTAAGTTCTGCAACGTGAGATTTTCCACATG
CGACAACCAGAAGAGCTGTATGAGCAACTGCAGCATCACAAGCATCTGCGAGAAGC
CACAAGAGGTCTGCGTGGCCGTCTGGAGAAAGAACGACGAGAACATCACTCTCGAG
ACTGTGTGTCACGACCCTAAGCTCCCATACCATGACTTCATCCTCGAGGATGCTGCC
TCCCCTAAATGCATTATGAAGGAGAAAAAAAAGCCCGGCGAGACATTCTTTATGTG
CAGCTGCTCCTCCGACGCCTGCAACGACAACATCATCTTTAGCGAAGAATACAACAC
TAGCAACCCAGATGGCGGCGGAGGATCCGGAGGAGGAGGCTCCGGAGGCGGAGGC
AGCATCCCACCACACGTGCAGAAGTCCGTCAACAACGACATGATTGTGACTGACAA
CAACGGCGCCGTGAAGTTCCCACAACTCTGCAAGTTTTGCAATGTGAGGTTCTCCAC
ATGTGACAACCAGAAAAGCTGCATGTCCAACTGCTCCATCACTAGCATCTGTGAGAA
ACCTCAAGAGGTCTGTGTGGCTGTGTGGAGGAAGAACGATGAGAACATCACACTCG
AGACAGTCTGCCACGACCCAAAGCTGCCATATCACGACTTCATTCTCGAGGACGCCG
CCAGCCCTAAGTGCATCATGAAGGAGAAGAAGAAGCCCGGCGAGACATTTTTCATG
TGTAGCTGCAGCTCCGATGCCTGTAACGACAATATTATCTTTAGCGAGGAGTATAAC
ACATCCAATCCAGACATTACATGCCCCCCTCCCATGAGCGTGGAGCACGCCGACATC
TGGGTGAAGAGCTATAGCCTCTACAGCCGGGAGAGGTATATCTGTAACAGCGGCTT
CAAGAGGAAGGCCGGCACCAGCAGCCTCACCGAGTGCGTGCTGAATAAGGCTACCA
ACGTGGCTCACTGGACAACACCCTCTTTAAAGTGCATCCGG
Exemplary Nucleic Acid Encoding an Exemplary Second Chimeric
Polypeptide
(SEQ ID NO: 35)
ATGAAGTGGGTGACCTTCATCAGCCTGCTGTTCCTGTTCTCCAGCGCCTACTCCATCC
CACCTCACGTGCAGAAAAGCGTCAATAATGACATGATCGTGACTGACAATAACGGC
GCCGTCAAGTTTCCACAGCTGTGTAAGTTCTGCAACGTGAGATTTTCCACATGCGAC
AACCAGAAGAGCTGTATGAGCAACTGCAGCATCACAAGCATCTGCGAGAAGCCACA
AGAGGTCTGCGTGGCCGTCTGGAGAAAGAACGACGAGAACATCACTCTCGAGACTG
TGTGTCACGACCCTAAGCTCCCATACCATGACTTCATCCTCGAGGATGCTGCCTCCC
CTAAATGCATTATGAAGGAGAAAAAAAAGCCCGGCGAGACATTCTTTATGTGCAGC
TGCTCCTCCGACGCCTGCAACGACAACATCATCTTTAGCGAAGAATACAACACTAG
CAACCCAGATGGCGGCGGAGGATCCGGAGGAGGAGGCTCCGGAGGCGGAGGCAGC
ATCCCACCACACGTGCAGAAGTCCGTCAACAACGACATGATTGTGACTGACAACAA
CGGCGCCGTGAAGTTCCCACAACTCTGCAAGTTTTGCAATGTGAGGTTCTCCACATG
TGACAACCAGAAAAGCTGCATGTCCAACTGCTCCATCACTAGCATCTGTGAGAAAC
CTCAAGAGGTCTGTGTGGCTGTGTGGAGGAAGAACGATGAGAACATCACACTCGAG
ACAGTCTGCCACGACCCAAAGCTGCCATATCACGACTTCATTCTCGAGGACGCCGCC
AGCCCTAAGTGCATCATGAAGGAGAAGAAGAAGCCCGGCGAGACATTTTTCATGTG
TAGCTGCAGCTCCGATGCCTGTAACGACAATATTATCTTTAGCGAGGAGTATAACAC
ATCCAATCCAGACATTACATGCCCCCCTCCCATGAGCGTGGAGCACGCCGACATCTG
GGTGAAGAGCTATAGCCTCTACAGCCGGGAGAGGTATATCTGTAACAGCGGCTTCA
AGAGGAAGGCCGGCACCAGCAGCCTCACCGAGTGCGTGCTGAATAAGGCTACCAAC
GTGGCTCACTGGACAACACCCTCTTTAAAGTGCATCCGG

### Compositions/Kits

Also provided herein are compositions (e.g., pharmaceutical compositions) that include at least one of any multi-chain chimeric polypeptides, any of the cells, or any of the nucleic acids described herein. In some embodiments, the compositions include at least one of any of the multi-chain chimeric polypeptides described herein. In some embodiments, the compositions include any of the immune cells (e.g., any of the immune cells described herein, e.g., any of the immune cells produced using any of the methods described herein).

In some embodiments, the pharmaceutical compositions are formulated for different routes of administration (e.g., intravenous, subcutaneous). In some embodiments, the pharmaceutical compositions can include a pharmaceutically acceptable carrier (e.g., phosphate buffered saline).

Single or multiple administrations of pharmaceutical compositions can be given to a subject in need thereof depending on for example: the dosage and frequency as required and tolerated by the subject. The formulation should provide a sufficient quantity of active agent to effectively treat, prevent or ameliorate conditions, diseases or symptoms.

Also provided herein are kits that include any of the multi-chain chimeric polypeptides, compositions, nucleic acids, or cells (e.g., immune cells) described herein. In some embodiments, the kits can include instructions for performing any of the methods described herein. In some embodiments, the kits can include at least one dose of any of the pharmaceutical compositions described herein.

### Nucleic Acids/Vectors

Also provided herein are nucleic acids that encode any of the multi-chain chimeric polypeptides described herein. In some embodiments, a first nucleic acid can encode the first chimeric polypeptide and a second nucleic acid can encode the second chimeric polypeptide. In some embodiments, a single nucleic acid can encode both the first chimeric polypeptide and the second chimeric polypeptide.

Also provided herein are vectors that include any of the nucleic acids encoding any of the multi-chain chimeric polypeptides described herein. In some embodiments, a first vector can include a nucleic acid encoding the first chimeric polypeptide and a second vector can include a nucleic acid encoding the second chimeric polypeptide. In some embodiments, a single vector can include a first nucleic acid encoding the first chimeric polypeptide and a second nucleic acid encoding the second chimeric polypeptide.

Any of the vectors described herein can be an expression vector. For example, an expression vector can include a promoter sequence operably linked to the sequence encoding the first chimeric polypeptide and the second chimeric polypeptide.

Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any GATEWAY® vectors. A vector can, e.g., include sufficient cis-acting elements for expression; other elements for expression can be supplied by the host mammalian cell or in an in vitro expression system. Skilled practitioners will be capable of selecting suitable vectors and mammalian cells for making any of the multi-chain chimeric polypeptides described herein.

### Cells

Also provided herein are cells (e.g., any of the exemplary cells described herein or known in the art) comprising any of the nucleic acids described herein that encode any of the multi-chain chimeric polypeptides described herein (e.g., encoding both the first and second chimeric polypeptides). Also provided herein are cells (e.g., any of the exemplary cells described herein or known in the art) comprising any of the nucleic acids described herein that encode any of the first chimeric polypeptides described herein. Also provided are cells (e.g., any of the exemplary cells described herein or known in the art) comprising any of the nucleic acids described herein that encode any of the second chimeric polypeptides described herein.

Also provided herein are cells (e.g., any of the exemplary cells described herein or known in the art) that include any of the vectors described herein that encode any of the multi-chain chimeric polypeptides described herein (e.g., encoding both the first and second chimeric polypeptides). Also provided herein are cells (e.g., any of the exemplary cells described herein or known in the art) that include any of the vectors described herein that encode any of the first chimeric polypeptides described herein. Also provided herein are cells (e.g., any of the exemplary cells described herein or known in the art) that include any of the vectors described herein that encode any of the second chimeric polypeptides described herein).

In some embodiments of any of the methods described herein, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary cells and human embryonic kidney cells (e.g., HEK293 cells).

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection.

### Methods of Producing Multi-Chain Chimeric Polypeptides

Also provided herein are methods of producing any of the multi-chain chimeric polypeptides described herein that include culturing any of the cells described herein in a culture medium under conditions sufficient to result in the production of the multi-chain chimeric polypeptide; and recovering the multi-chain chimeric polypeptide from the cell and/or the culture medium.

Also provided herein are method of producing any of the multi-chain chimeric polypeptides described herein that include: culturing any of cells described herein in a first culture medium under conditions sufficient to result in the production of the first chimeric polypeptide; recovering the first chimeric polypeptide from the cell and/or the first culture medium; culturing any of the cells described herein in a second culture medium under conditions sufficient to result in the production of the second chimeric polypeptide; recovering the second chimeric polypeptide from the cell and/or the second culture medium; and combining (e.g., mixing) the recovered first chimeric polypeptide and the recovered second chimeric polypeptide to form the multi-chain chimeric polypeptide (e.g., any of the multi-chain chimeric polypeptides described herein).

The recovery of the multi-chain chimeric polypeptide, the first chimeric polypeptide, or the second chimeric polypeptide from a cell (e.g., a eukaryotic cell) can be performed using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

Methods of culturing cells are well known in the art. Cells can be maintained in vitro under conditions that favor proliferation, differentiation and growth. Briefly, cells can be cultured by contacting a cell (e.g., any cell) with a cell culture medium that includes the necessary growth factors and supplements to support cell viability and growth.

Also provided herein are multi-chain chimeric polypeptides (e.g., any of the multi-chain chimeric polypeptides described herein), first chimeric polypeptides (e.g., any of the first chimeric polypeptides), or second chimeric polypeptides (e.g., any of the second chimeric polypeptides described herein) produced by any of the methods described herein.

### Methods of Treatment

Also provided herein are methods of treating bronchopulmonary dysplasia in a subject (e.g., any of the exemplary subjects described herein or known in the art) that include administering to the subject a therapeutically effective amount of any of the multi-chain chimeric polypeptides described herein or any of the compositions (e.g., pharmaceutical compositions) described herein.

In some examples of these methods, the subject has been diagnosed or identified as having bronchopulmonary dysplasia. Subjects can be identified as having bronchopulmonary dysplasia using chest X-ray, blood tests, and echocardiogram.

In some examples, the method results in an increase (e.g., at least a 1% increase, at least a 10% increase, at least a 15% increase, at least a 20% increase, at least a 25% increase, at least a 30% increase, at least a 35% increase, or at least a 40% increase) (e.g., about a 1% increase to about a 40% increase, about a 1% increase to about a 35% increase, about a 1% increase to about a 30% increase, about a 1% increase to about a 25% increase, about a 1% increase to about a 20% increase, about a 1% increase to about a 15% increase, about a 1% increase to about a 10% increase, about a 1% increase to about a 8% increase, about a 1% increase to about a 6% increase, about a 1% increase to about a 4% increase, about a 2% increase to about a 40% increase, about a 2% increase to about a 35% increase, about a 2% increase to about a 30% increase, about a 2% increase to about a 25% increase, about a 2% increase to about a 20% increase, about a 2% increase to about a 15% increase, about a 2% increase to about a 10% increase, about a 2% increase to about a 8% increase, about a 2% increase to about a 6% increase, about a 2% increase to about a 4% increase, about a 4% increase to about a 40% increase, about a 4% increase to about a 35% increase, about a 4% increase to about a 30% increase, about a 4% increase to about a 25% increase, about a 4% increase to about a 20% increase, about a 4% increase to about a 15% increase, about a 4% increase to about a 10% increase, about a 4% increase to about a 8% increase, about a 4% increase to about a 6% increase, about a 6% increase to about a 40% increase, about a 6% increase to about a 35% increase, about a 6% increase to about a 30% increase, about a 6% increase to about a 25% increase, about a 6% increase to about a 20% increase, about a 6% increase to about a 15% increase, about a 6% increase to about a 10% increase, about a 6% increase to about a 8% increase, about a 8% increase to about a 40% increase, about a 8% increase to about a 35% increase, about a 8% increase to about a 30% increase, about a 8% increase to about a 25% increase, about a 8% increase to about a 20% increase, about a 8% increase to about a 15% increase, about a 8% increase to about a 10% increase, about a 10% increase to about a 40% increase, about a 10% increase to about a 35% increase, about a 10% increase to about a 30% increase, about a 10% increase to about a 25% increase, about a 10% increase to about a 20% increase, about a 10% increase to about a 15% increase, about a 15% increase to about a 40% increase, about a 15% increase to about a 35% increase, about a 15% increase to about a 30% increase, about a 15% increase to about a 25% increase, about a 15% increase to about a 20% increase, about a 20% increase to about a 40% increase, about a 20% increase to about a 35% increase, about a 20% increase to about a 30% increase, about a 20% increase to about a 25% increase, about a 25% increase to about a 40% increase, about a 25% increase to about a 35% increase, about a 25% increase to about a 30% increase, about a 30% increase to about a 40% increase, about a 30% increase to about a 35% increase, or about a 35% increase to about a 40% increase) in the heart mass in the subject, e.g., as compared to the heart mass of the subject prior to treatment.

In some examples, the method results in an increase (e.g., at least a 1% increase, at least a 10% increase, at least a 15% increase, at least a 20% increase, at least a 25% increase, at least a 30% increase, at least a 35% increase, or at least a 40% increase) (e.g., about a 1% increase to about a 40% increase, about a 1% increase to about a 35% increase, about a 1% increase to about a 30% increase, about a 1% increase to about a 25% increase, about a 1% increase to about a 20% increase, about a 1% increase to about a 15% increase, about a 1% increase to about a 10% increase, about a 1% increase to about a 8% increase, about a 1% increase to about a 6% increase, about a 1% increase to about a 4% increase, about a 2% increase to about a 40% increase, about a 2% increase to about a 35% increase, about a 2% increase to about a 30% increase, about a 2% increase to about a 25% increase, about a 2% increase to about a 20% increase, about a 2% increase to about a 15% increase, about a 2% increase to about a 10% increase, about a 2% increase to about a 8% increase, about a 2% increase to about a 6% increase, about a 2% increase to about a 4% increase, about a 4% increase to about a 40% increase, about a 4% increase to about a 35% increase, about a 4% increase to about a 30% increase, about a 4% increase to about a 25% increase, about a 4% increase to about a 20% increase, about a 4% increase to about a 15% increase, about a 4% increase to about a 10% increase, about a 4% increase to about a 8% increase, about a 4% increase to about a 6% increase, about a 6% increase to about a 40% increase, about a 6% increase to about a 35% increase, about a 6% increase to about a 30% increase, about a 6% increase to about a 25% increase, about a 6% increase to about a 20% increase, about a 6% increase to about a 15% increase, about a 6% increase to about a 10% increase, about a 6% increase to about a 8% increase, about a 8% increase to about a 40% increase, about a 8% increase to about a 35% increase, about a 8% increase to about a 30% increase, about a 8% increase to about a 25% increase, about a 8% increase to about a 20% increase, about a 8% increase to about a 15% increase, about a 8% increase to about a 10% increase, about a 10% increase to about a 40% increase, about a 10% increase to about a 35% increase, about a 10% increase to about a 30% increase, about a 10% increase to about a 25% increase, about a 10% increase to about a 20% increase, about a 10% increase to about a 15% increase, about a 15% increase to about a 40% increase, about a 15% increase to about a 35% increase, about a 15% increase to about a 30% increase, about a 15% increase to about a 25% increase, about a 15% increase to about a 20% increase, about a 20% increase to about a 40% increase, about a 20% increase to about a 35% increase, about a 20% increase to about a 30% increase, about a 20% increase to about a 25% increase, about a 25% increase to about a 40% increase, about a 25% increase to about a 35% increase, about a 25% increase to about a 30% increase, about a 30% increase to about a 40% increase, about a 30% increase to about a 35% increase, or about a 35% increase to about a 40% increase) in the ratio of heart mass to body mass in a subject, e.g., as compared to the ratio of heart mass to body mass in the subject prior to treatment.

In some examples, the method results in an increase (e.g., at least a 1% increase, at least a 10% increase, at least a 15% increase, at least a 20% increase, at least a 25% increase, at least a 30% increase, at least a 35% increase, or at least a 40% increase) (e.g., about a 1% increase to about a 40% increase, about a 1% increase to about a 35% increase, about a 1% increase to about a 30% increase, about a 1% increase to about a 25% increase, about a 1% increase to about a 20% increase, about a 1% increase to about a 15% increase, about a 1% increase to about a 10% increase, about a 1% increase to about a 8% increase, about a 1% increase to about a 6% increase, about a 1% increase to about a 4% increase, about a 2% increase to about a 40% increase, about a 2% increase to about a 35% increase, about a 2% increase to about a 30% increase, about a 2% increase to about a 25% increase, about a 2% increase to about a 20% increase, about a 2% increase to about a 15% increase, about a 2% increase to about a 10% increase, about a 2% increase to about a 8% increase, about a 2% increase to about a 6% increase, about a 2% increase to about a 4% increase, about a 4% increase to about a 40% increase, about a 4% increase to about a 35% increase, about a 4% increase to about a 30% increase, about a 4% increase to about a 25% increase, about a 4% increase to about a 20% increase, about a 4% increase to about a 15% increase, about a 4% increase to about a 10% increase, about a 4% increase to about a 8% increase, about a 4% increase to about a 6% increase, about a 6% increase to about a 40% increase, about a 6% increase to about a 35% increase, about a 6% increase to about a 30% increase, about a 6% increase to about a 25% increase, about a 6% increase to about a 20% increase, about a 6% increase to about a 15% increase, about a 6% increase to about a 10% increase, about a 6% increase to about a 8% increase, about a 8% increase to about a 40% increase, about a 8% increase to about a 35% increase, about a 8% increase to about a 30% increase, about a 8% increase to about a 25% increase, about a 8% increase to about a 20% increase, about a 8% increase to about a 15% increase, about a 8% increase to about a 10% increase, about a 10% increase to about a 40% increase, about a 10% increase to about a 35% increase, about a 10% increase to about a 30% increase, about a 10% increase to about a 25% increase, about a 10% increase to about a 20% increase, about a 10% increase to about a 15% increase, about a 15% increase to about a 40% increase, about a 15% increase to about a 35% increase, about a 15% increase to about a 30% increase, about a 15% increase to about a 25% increase, about a 15% increase to about a 20% increase, about a 20% increase to about a 40% increase, about a 20% increase to about a 35% increase, about a 20% increase to about a 30% increase, about a 20% increase to about a 25% increase, about a 25% increase to about a 40% increase, about a 25% increase to about a 35% increase, about a 25% increase to about a 30% increase, about a 30% increase to about a 40% increase, about a 30% increase to about a 35% increase, or about a 35% increase to about a 40% increase) in body mass of the subject, e.g., as compared to a similar subject having bronchopulmonary dysplasia not receiving the treatment.

In some examples, the method results in an increase (e.g., at least a 1% increase, at least a 10% increase, at least a 15% increase, at least a 20% increase, at least a 25% increase, at least a 30% increase, at least a 35% increase, at least a 40% increase, at least a 45% increase, or at least a 50% increase) (e.g., about a 1% increase to about a 50% increase, about a 1% increase to about a 45% increase, about a 1% increase to about a 40% increase, about a 1% increase to about a 35% increase, about a 1% increase to about a 30% increase, about a 1% increase to about a 25% increase, about a 1% increase to about a 20% increase, about a 1% increase to about a 15% increase, about a 1% increase to about a 10% increase, about a 1% increase to about a 8% increase, about a 1% increase to about a 6% increase, about a 1% increase to about a 4% increase, about a 2% increase to about a 50% increase, about a 2% increase to about a 45% increase, about a 2% increase to about a 40% increase, about a 2% increase to about a 35% increase, about a 2% increase to about a 30% increase, about a 2% increase to about a 25% increase, about a 2% increase to about a 20% increase, about a 2% increase to about a 15% increase, about a 2% increase to about a 10% increase, about a 2% increase to about a 8% increase, about a 2% increase to about a 6% increase, about a 2% increase to about a 4% increase, about a 4% increase to about a 50% increase, about a 4% increase to about a 45% increase, about a 4% increase to about a 40% increase, about a 4% increase to about a 35% increase, about a 4% increase to about a 30% increase, about a 4% increase to about a 25% increase, about a 4% increase to about a 20% increase, about a 4% increase to about a 15% increase, about a 4% increase to about a 10% increase, about a 4% increase to about a 8% increase, about a 4% increase to about a 6% increase, about a 6% increase to about a 50% increase, about a 6% increase to about a 45% increase, about a 6% increase to about a 40% increase, about a 6% increase to about a 35% increase, about a 6% increase to about a 30% increase, about a 6% increase to about a 25% increase, about a 6% increase to about a 20% increase, about a 6% increase to about a 15% increase, about a 6% increase to about a 10% increase, about a 6% increase to about a 8% increase, about a 8% increase to about a 50% increase, about a 8% increase to about a 45% increase, about a 8% increase to about a 40% increase, about a 8% increase to about a 35% increase, about a 8% increase to about a 30% increase, about a 8% increase to about a 25% increase, about a 8% increase to about a 20% increase, about a 8% increase to about a 15% increase, about a 8% increase to about a 10% increase, about a 10% increase to about a 50% increase, about a 10% increase to about a 45% increase, about a 10% increase to about a 40% increase, about a 10% increase to about a 35% increase, about a 10% increase to about a 30% increase, about a 10% increase to about a 25% increase, about a 10% increase to about a 20% increase, about a 10% increase to about a 15% increase, about a 15% increase to about a 50% increase, about a 15% increase to about a 45% increase, about a 15% increase to about a 40% increase, about a 15% increase to about a 35% increase, about a 15% increase to about a 30% increase, about a 15% increase to about a 25% increase, about a 15% increase to about a 20% increase, about a 20% increase to about a 50% increase, about a 20% increase to about a 45% increase, about a 20% increase to about a 40% increase, about a 20% increase to about a 35% increase, about a 20% increase to about a 30% increase, about a 20% increase to about a 25% increase, about a 25% increase to about a 50% increase, about a 25% increase to about a 45% increase, about a 25% increase to about a 40% increase, about a 25% increase to about a 35% increase, about a 25% increase to about a 30% increase, about a 30% increase to about a 50% increase, about a 30% increase to about a 45% increase, about a 30% increase to about a 40% increase, about a 30% increase to about a 35% increase, about a 35% increase to about a 50% increase, about a 35% increase to about a 45% increase, about a 35% increase to about a 40% increase, about a 40% increase to about a 50% increase, about a 40% increase to about a 45% increase, or about a 45% increase to about a 50% increase) in the oxygen levels in the blood of the subject, e.g., as compared to the oxygen levels in the subject prior to treatment.

The term “subject” refers to any mammal. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine or primate animals) may be employed.

In some embodiments, the subject is 1 week to 10 years old (e.g., about 1 week to about 9 years old, about 1 week to about 8 years old, about 1 week to about 7 years old, about 1 week to about 6 years old, about 1 week to about 5 years old, about 1 week to about 4 years old, about 1 week to about 3 years old, about 1 week to about 2 years old, about 1 week to about 1 year old, about 1 week to about 10 months old, about 1 week to about 8 months old, about 1 week to about 6 months old, about 1 week to about 4 months old, about 1 week to about 2 months old, about 1 week to about 6 weeks old, about 1 week to about 1 month old, about 1 week to about 2 weeks old, about 2 weeks to about 10 years old, about 2 weeks to about 9 years old, about 2 weeks to about 8 years old, about 2 weeks to about 7 years old, about 2 weeks to about 6 years old, about 2 weeks to about 5 years old, about 2 weeks to about 4 years old, about 2 weeks to about 3 years old, about 2 weeks to about 2 years old, about 2 weeks to about 1 year old, about 2 weeks to about 10 months old, about 2 weeks to about 8 months old, about 2 weeks to about 6 months old, about 2 weeks to about 4 months old, about 2 weeks to about 2 months old, about 2 weeks to about 6 weeks old, about 2 weeks to about 1 month old, about 1 month to about 10 years old, about 1 month to about 9 years old, about 1 month to about 8 years old, about 1 month to about 7 years old, about 1 month to about 6 years old, about 1 month to about 5 years old, about 1 month to about 4 years old, about 1 month to about 3 years old, about 1 month to about 2 years old, about 1 month to about 1 year old, about 1 month to about 10 months old, about 1 month to about 8 months old, about 1 month to about 6 months old, about 1 month to about 4 months old, about 1 month to about 2 months old, about 1 month to about 6 weeks old, about 6 weeks to about 10 years old, about 6 weeks to about 9 years old, about 6 weeks to about 8 years old, about 6 weeks to about 7 years old, about 6 weeks to about 6 years old, about 6 weeks to about 5 years old, about 6 weeks to about 4 years old, about 6 weeks to about 3 years old, about 6 weeks to about 2 years old, about 6 weeks to about 1 year old, about 6 weeks to about 10 months old, about 6 weeks to about 8 months old, about 6 weeks to about 6 months old, about 6 weeks to about 4 months old, about 6 weeks to about 2 months old, about 2 months to about 10 years old, about 2 months to about 9 years old, about 2 months to about 8 years old, about 2 months to about 7 years old, about 2 months to about 6 years old, about 2 months to about 5 years old, about 2 months to about 4 years old, about 2 months to about 3 years old, about 2 months to about 2 years old, about 2 months to about 1 year old, about 2 months to about 10 months old, about 2 months to about 8 months old, about 2 months to about 6 months old, about 2 months to about 4 months old, about 4 months to about 10 years old, about 4 months to about 9 years old, about 4 months to about 8 years old, about 4 months to about 7 years old, about 4 months to about 6 years old, about 4 months to about 5 years old, about 4 months to about 4 years old, about 4 months to about 3 years old, about 4 months to about 2 years old, about 4 months to about 1 year old, about 4 months to about 10 months old, about 4 months to about 8 months old, about 4 months to about 6 months old, about 6 months to about 10 years old, about 6 months to about 9 years old, about 6 months to about 8 years old, about 6 months to about 7 years old, about 6 months to about 6 years old, about 6 months to about 5 years old, about 6 months to about 4 years old, about 6 months to about 3 years old, about 6 months to about 2 years old, about 6 months to about 1 year old, about 6 months to about 10 months old, about 6 months to about 8 months old, about 8 months to about 10 years old, about 8 months to about 9 years old, about 8 months to about 8 years old, about 8 months to about 7 years old, about 8 months to about 6 years old, about 8 months to about 5 years old, about 8 months to about 4 years old, about 8 months to about 3 years old, about 8 months to about 2 years old, about 8 months to about 1 year old, about 8 months to about 10 months old, about 10 months to about 10 years old, about 10 months to about 9 years old, about 10 months to about 8 years old, about 10 months to about 7 years old, about 10 months to about 6 years old, about 10 months to about 5 years old, about 10 months to about 4 years old, about 10 months to about 3 years old, about 10 months to about 2 years old, about 10 months to about 1 year old, about 1 year to about 10 years old, about 1 year to about 9 years old, about 1 year to about 8 years old, about 1 year to about 7 years old, about 1 year to about 6 years old, about 1 year to about 5 years old, about 1 year to about 4 years old, about 1 year to about 3 years old, about 1 year to about 2 years old, about 2 years to about 10 years old, about 2 years to about 9 years old, about 2 years to about 8 years old, about 2 years to about 7 years old, about 2 years to about 6 years old, about 2 years to about 5 years old, about 2 years to about 4 years old, about 2 years to about 3 years old, about 3 years to about 10 years old, about 3 years to about 9 years old, about 3 years to about 8 years old, about 3 years to about 7 years old, about 3 years to about 6 years old, about 3 years to about 5 years old, about 3 years to about 4 years old, about 4 years to about 10 years old, about 4 years to about 9 years old, about 4 years to about 8 years old, about 4 years to about 7 years old, about 4 years to about 6 years old, about 4 years to about 5 years old, about 5 years to about 10 years old, about 5 years to about 9 years old, about 5 years to about 8 years old, about 5 years to about 7 years old, about 5 years to about 6 years old, about 6 years to about 10 years old, about 6 years to about 9 years old, about 6 years to about 8 years old, about 6 years to about 7 years old, about 7 years to about 10 years old, about 7 years to about 9 years old, about 7 years to about 8 years old, about 8 years to about 10 years old, about 8 years to about 9 years old, or about 9 years to about 10 years old).

### EXAMPLES

The invention is further described in the following examples, which do not limit the scope of the invention described in the claims.

### Example 1: TGFRt15-TGFRs Fusion Protein Generation and Characterization

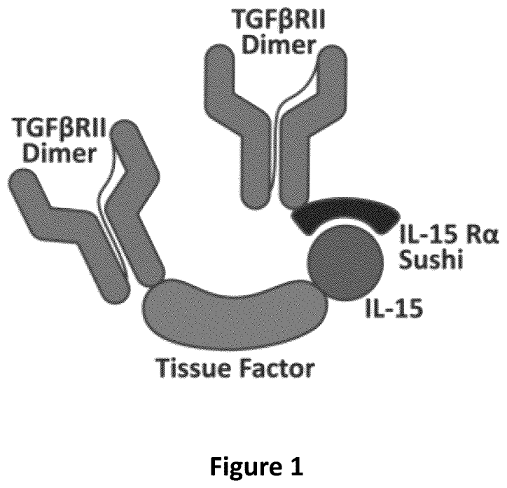

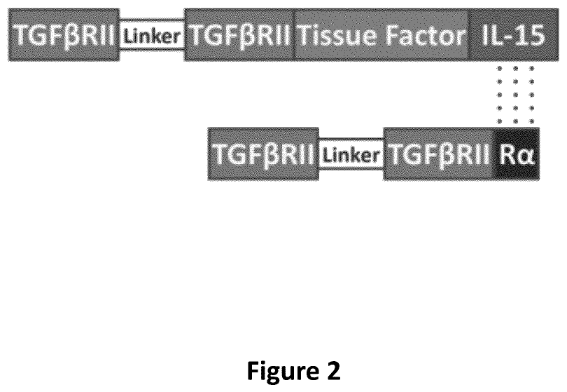

A fusion protein complex was generated comprising of TGFβ Receptor II/IL-15RαSu and TGFβ Receptor II/TF/IL-15 fusion proteins (FIG. 1 and FIG. 2). The human TGFβ Receptor II (Ile24-Asp159), tissue factor 219, and IL-15 sequences were obtained from the UniProt website and DNA for these sequences was synthesized by Genewiz. Specifically, a construct was made linking two TGFβ Receptor II sequences with a G4S(3) linker to generate a single chain version of TGFβ Receptor II and then directly linking to the N-terminus coding region of tissue factor 219 followed by the N-terminus coding region of IL-15.

The nucleic acid and protein sequences of a construct comprising two TGFβ Receptor II linked to the N-terminus of tissue factor 219 following with the N-terminus of IL-15 are shown below.

The nucleic acid sequence of the two TGFβ Receptor II/TF/IL-15 construct (including signal peptide sequence) is as follows:

(Signal peptide)
ATGAAGTGGGTGACCTTCATCAGCCTGCTGTTCCTGTTCTCCAGCGCCTACTC
C
(Two Human TGFβ Receptor II fragments)
ATCCCCCCCCATGTGCAAAAGAGCGTGAACAACGATATGATCGTGACCGACA
ACAACGGCGCCGTGAAGTTTCCCCAGCTCTGCAAGTTCTGCGATGTCAGGTTCAGCA
CCTGCGATAATCAGAAGTCCTGCATGTCCAACTGCAGCATCACCTCCATCTGCGAGA
AGCCCCAAGAAGTGTGCGTGGCCGTGTGGCGGAAAAATGACGAGAACATCACCCTG
GAGACCGTGTGTCACGACCCCAAGCTCCCTTATCACGACTTCATTCTGGAGGACGCT
GCCTCCCCCAAATGCATCATGAAGGAGAAGAAGAAGCCCGGAGAGACCTTCTTTAT
GTGTTCCTGTAGCAGCGACGAGTGTAACGACAACATCATCTTCAGCGAAGAGTACA
ACACCAGCAACCCTGATGGAGGTGGCGGATCCGGAGGTGGAGGTTCTGGTGGAGGT
GGGAGTATTCCTCCCCACGTGCAGAAGAGCGTGAATAATGACATGATCGTGACCGA
TAACAATGGCGCCGTGAAATTTCCCCAGCTGTGCAAATTCTGCGATGTGAGGTTTTC
CACCTGCGACAACCAGAAGTCCTGTATGAGCAACTGCTCCATCACCTCCATCTGTGA
GAAGCCTCAGGAGGTGTGCGTGGCTGTCTGGCGGAAGAATGACGAGAATATCACCC
TGGAAACCGTCTGCCACGATCCCAAGCTGCCCTACCACGATTTCATCCTGGAAGACG
CCGCCAGCCCTAAGTGCATCATGAAAGAGAAAAAGAAGCCTGGCGAGACCTTTTTC
ATGTGCTCCTGCAGCAGCGACGAATGCAACGACAATATCATCTTTAGCGAGGAATA
CAATACCAGCAACCCCGAC
(Human Tissue Factor 219)
AGCGGCACAACCAACACAGTCGCTGCCTATAACCTCACTTGGAAGAGCACCA
ACTTCAAAACCATCCTCGAATGGGAACCCAAACCCGTTAACCAAGTTTACACCGTGC
AGATCAGCACCAAGTCCGGCGACTGGAAGTCCAAATGTTTCTATACCACCGACACC
GAGTGCGATCTCACCGATGAGATCGTGAAAGATGTGAAACAGACCTACCTCGCCCG
GGTGTTTAGCTACCCCGCCGGCAATGTGGAGAGCACTGGTTCCGCTGGCGAGCCTTT
ATACGAGAACAGCCCCGAATTTACCCCTTACCTCGAGACCAATTTAGGACAGCCCAC
CATCCAAAGCTTTGAGCAAGTTGGCACAAAGGTGAATGTGACAGTGGAGGACGAGC
GGACTTTAGTGCGGCGGAACAACACCTTTCTCAGCCTCCGGGATGTGTTCGGCAAAG
ATTTAATCTACACACTGTATTACTGGAAGTCCTCTTCCTCCGGCAAGAAGACAGCTA
AAACCAACACAAACGAGTTTTTAATCGACGTGGATAAAGGCGAAAACTACTGTTTC
AGCGTGCAAGCTGTGATCCCCTCCCGGACCGTGAATAGGAAAAGCACCGATAGCCC
CGTTGAGTGCATGGGCCAAGAAAAGGGCGAGTTCCGGGAG
(Human IL-15)
(SEQ ID NO: 11)
AACTGGGTGAACGTCATCAGCGATTTAAAGAAGATCGAAGATTTAATTCAGT
CCATGCATATCGACGCCACTTTATACACAGAATCCGACGTGCACCCCTCTTGTAAGG
TGACCGCCATGAAATGTTTTTTACTGGAGCTGCAAGTTATCTCTTTAGAGAGCGGAG
ACGCTAGCATCCACGACACCGTGGAGAATTTAATCATTTTAGCCAATAACTCTTTAT
CCAGCAACGGCAACGTGACAGAGTCCGGCTGCAAGGAGTGCGAAGAGCTGGAGGA
GAAGAACATCAAGGAGTTTCTGCAATCCTTTGTGCACATTGTCCAGATGTTCATCAA
TACCTCC.

The amino acid sequence of TGFβ Receptor II/TF/IL-15 fusion protein (including the leader sequence) is as follows:

(Signal peptide)
MKWVTFISLLFLFSSAYS
(Human TGFβ Receptor II)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD
(Human Tissue Factor 219)
SGTTNTVAAYNLTWKSTNFKTILEWEPKPVNQVYTVQISTKSGDWKSKC
FYTTDTECDLTDEIVKDVKQTYLARVFSYPAGNVESTGSAGEPLYENSP
EFTPYLETNLGQPTIQSFEQVGTKVNVTVEDERTLVRRNNTFLSLRDVF
GKDLIYTLYYWKSSSSGKKTAKTNTNEFLIDVDKGENYCFSVQAVIPSR
TVNRKSTDSPVECMGQEKGEFRE
(Human IL-15)
(SEQ ID NO: 10)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS.

Constructs were also made by attaching two TGFβ Receptor II directly to the IL-15RαSu chain which was synthesized by Genewiz. The nucleic acid and protein sequences of a construct comprising the TGFβ Receptor II linked to the N-terminus of IL-15RαSu are shown below.

The nucleic acid sequence of the TGFβ Receptor II/IL-15 RαSu construct (including signal peptide sequence) is as follows:

(Signal peptide)
ATGAAGTGGGTGACCTTCATCAGCCTGCTGTTCCTGTTCTCCAGCGCCT
ACTCC
(Two human TGFβ Receptor II fragments)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCTCCATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGAC
(Human IL-15Rα sushi domain)
(SEQ ID NO: 19)
ATTACATGCCCCCCTCCCATGAGCGTGGAGCACGCCGACATCTGGGTGA
AGAGCTATAGCCTCTACAGCCGGGAGAGGTATATCTGTAACAGCGGCTT
CAAGAGGAAGGCCGGCACCAGCAGCCTCACCGAGTGCGTGCTGAATAAG
GCTACCAACGTGGCTCACTGGACAACACCCTCTTTAAAGTGCATCCGG.

The amino acid sequence of the two TGFβ Receptor II/IL-15RαSu construct (including signal peptide sequence) is as follows:

(Signal peptide)
MKWVTFISLLFLFSSAYS
(Two human TGFβ Receptor II extra-cellular
domains)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD
(Human IL-15Rα sushi domain)
(SEQ ID NO: 18)
ITCPPPMSVEHADIWVKSYSLYSRERYICNSGFKRKAGTSSLTECVLNK
ATNVAHWTTPSLKCIR.

In some cases, the leader peptide is cleaved from the intact polypeptide to generate the mature form that may be soluble or secreted.

The TGFβR/IL-15RαSu and TGFβR/TF/IL-15 constructs were cloned into a modified retrovirus expression vectors as described previously (Hughes M S, Yu Y Y, Dudley M E, Zheng Z, Robbins P F, Li Y, et al. Transfer of a TCR gene derived from a patient with a marked antitumor response conveys highly active T-cell effector functions. Hum Gene Ther 2005; 16:457-72), and the expression vectors were transfected into CHO-K1 cells. Co-expression of the two constructs in CHO-K1 cells allowed for formation and secretion of the soluble TGFβR/TF/IL-15:TGFβR/IL-15RαSu protein complex (referred to as TGFRt15-TGFRs), which can be purified by anti-TF IgG1 affinity and other chromatography methods.

### Example 2: Generation of TGFRt15*-TGFRs

A fusion protein complex was generated comprising of TGFR/IL15RαSu and TGFR/TF/IL-15D8N fusion proteins. The human TGF-β receptor (TGFR), IL-15 alpha receptor sushi domain (IL15RαSu), tissue factor (TF) and IL-15 with D8N mutant (IL15D8N) sequences were obtained from the GenBank website and DNA fragments for these sequences were synthesized by Genewiz. Specifically, a construct was made linking the TGFR sequence to the N-terminus coding region of IL15RαSu and the TGFR sequence to the N-terminus of tissue factor 219 followed by the N-terminus coding region of IL-15D8N.

The nucleic acid sequence of the TGFR/IL15RαSu construct (including signal peptide sequence) is as follows:

(Signal peptide)
ATGAAGTGGGTGACCTTCATCAGCCTGCTGTTCCTGTTCTCCAGCGCCT
ACTCC
(Single chain Human TGF-beta Receptor II
homodimer)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCTCCATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGAC
(Sushi domain of IL15 receptor alpha chain)
(SEQ ID NO: 19)
ATTACATGCCCCCCTCCCATGAGCGTGGAGCACGCCGACATCTGGGTGA
AGAGCTATAGCCTCTACAGCCGGGAGAGGTATATCTGTAACAGCGGCTT
CAAGAGGAAGGCCGGCACCAGCAGCCTCACCGAGTGCGTGCTGAATAAG
GCTACCAACGTGGCTCACTGGACAACACCCTCTTTAAAGTGCATCCGG.

The nucleic acid sequence of the TGFR/TF/IL15D8N construct (including signal peptide sequence) is as follows:

(Signal peptide)
ATGGGAGTGAAAGTTCTTTTTGCCCTTATTTGTATTGCTGTGGCCGAGGCC
(Single chain Human TGF-beta Receptor II homodimer)
ATCCCACCGCACGTTCAGAAGTCGGTGAATAACGACATGATAGTCACTGACA
ACAACGGTGCAGTCAAGTTTCCACAACTGTGTAAATTTTGTGATGTGAGATTTTCCA
CCTGTGACAACCAGAAATCCTGCATGAGCAACTGCAGCATCACCTCCATCTGTGAGA
AGCCACAGGAAGTCTGTGTGGCTGTATGGAGAAAGAATGACGAGAACATAACACTA
GAGACAGTTTGCCATGACCCCAAGCTCCCCTACCATGACTTTATTCTGGAAGATGCT
GCTTCTCCAAAGTGCATTATGAAGGAAAAAAAAAAGCCTGGTGAGACTTTCTTCATG
TGTTCCTGTAGCTCTGATGAGTGCAATGACAACATCATCTTCTCAGAAGAATATAAC
ACCAGCAATCCTGACGGAGGTGGCGGATCCGGAGGTGGAGGTTCTGGTGGAGGTGG
GAGTATTCCTCCCCACGTGCAGAAGAGCGTGAATAATGACATGATCGTGACCGATA
ACAATGGCGCCGTGAAATTTCCCCAGCTGTGCAAATTCTGCGATGTGAGGTTTTCCA
CCTGCGACAACCAGAAGTCCTGTATGAGCAACTGCTCCATCACCTCCATCTGTGAGA
AGCCTCAGGAGGTGTGCGTGGCTGTCTGGCGGAAGAATGACGAGAATATCACCCTG
GAAACCGTCTGCCACGATCCCAAGCTGCCCTACCACGATTTCATCCTGGAAGACGCC
GCCAGCCCTAAGTGCATCATGAAAGAGAAAAAGAAGCCTGGCGAGACCTTTTTCAT
GTGCTCCTGCAGCAGCGACGAATGCAACGACAATATCATCTTTAGCGAGGAATACA
ATACCAGCAACCCCGAC
(Human Tissue Factor 219)
TCAGGCACTACAAATACTGTGGCAGCATATAATTTAACTTGGAAATCAACTA
ATTTCAAGACAATTTTGGAGTGGGAACCCAAACCCGTCAATCAAGTCTACACTGTTC
AAATAAGCACTAAGTCAGGAGATTGGAAAAGCAAATGCTTTTACACAACAGACACA
GAGTGTGACCTCACCGACGAGATTGTGAAGGATGTGAAGCAGACGTACTTGGCACG
GGTCTTCTCCTACCCGGCAGGGAATGTGGAGAGCACCGGTTCTGCTGGGGAGCCTCT
GTATGAGAACTCCCCAGAGTTCACACCTTACCTGGAGACAAACCTCGGACAGCCAA
CAATTCAGAGTTTTGAACAGGTGGGAACAAAAGTGAATGTGACCGTAGAAGATGAA
CGGACTTTAGTCAGAAGGAACAACACTTTCCTAAGCCTCCGGGATGTTTTTGGCAAG
GACTTAATTTATACACTTTATTATTGGAAATCTTCAAGTTCAGGAAAGAAAACAGCC
AAAACAAACACTAATGAGTTTTTGATTGATGTGGATAAAGGAGAAAACTACTGTTTC
AGTGTTCAAGCAGTGATTCCCTCCCGAACAGTTAACCGGAAGAGTACAGACAGCCC
GGTAGAGTGTATGGGCCAGGAGAAAGGGGAATTCAGAGAA
(Human IL-15D8N)
(SEQ ID NO: 15)
AACTGGGTGAATGTAATAAGTAATTTGAAAAAAATTGAAGATCTTATTCAAT
CTATGCATATTGATGCTACTTTATATACGGAAAGTGATGTTCACCCCAGTTGCAAAG
TAACAGCAATGAAGTGCTTTCTCTTGGAGTTACAAGTTATTTCACTTGAGTCCGGAG
ATGCAAGTATTCATGATACAGTAGAAAATCTGATCATCCTAGCAAACAACAGTTTGT
CTTCTAATGGGAATGTAACAGAATCTGGATGCAAAGAATGTGAGGAACTGGAGGAA
AAAAATATTAAAGAATTTTTGCAGAGTTTTGTACATATTGTCCAAATGTTCATCAAC
ACTTCT.

The amino acid sequence of TGFR/IL15RαSu fusion protein (including signal peptide sequence) is as follows:

(Signal peptide)
MKWVTFISLLFLFSSAYS
(Single chain Human TGF-beta Receptor II
homodimer)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD
(Human IL-15 receptor α sushi domain)
(SEQ ID NO: 18)
ITCPPPMSVEHADIWVKSYSLYSRERYICNSGFKRKAGTSSLTECVLNK
ATNVAHWTTPSLKCIR.

The amino acid sequence of TGFR/TF/IL15D8N fusion protein (including signal peptide sequence) is as follows:

(Signal peptide)
MGVKVLFALICIAVAEA
(Single chain Human TGF-beta Receptor II
homodimer)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD
(Tissue factor)
SGTTNTVAAYNLTWKSTNFKTILEWEPKPVNQVYTVQISTKSGDWKSKC
FYTTDTECDLTDEIVKDVKQTYLARVFSYPAGNVESTGSAGEPLYENSP
EFTPYLETNLGQPTIQSFEQVGTKVNVTVEDERTLVRRNNTFLSLRDVF
GKDLIYTLYYWKSSSSGKKTAKTNTNEFLIDVDKGENYCFSVQAVIPSR
TVNRKSTDSPVECMGQEKGEFRE
(IL-15D8N)
(SEQ ID NO: 14)
NWVNVISNLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS.

The TGFR/IL15RαSu and TGFR/TF/IL-15D8N constructs were cloned into a modified retrovirus expression vectors as described previously (Hughes M S, Yu Y Y, Dudley M E, Zheng Z, Robbins P F, Li Y, et al). The expression vectors were transfected into CHO-K1 cells. Co-expression of the two constructs in CHO-K1 cells allowed for formation and secretion of the soluble TGFR/IL15RαSu-TGFR/TF/IL-15D8N protein complex (referred to as TGFRt15*-TGFRs), which can be purified by anti-TF antibody affinity.

### Example 3: TGFRt15-TGFRs Treatment Improves Airway Patterning and Postnatal Development in a Mouse Model of Hyperoxia-Induced BPD

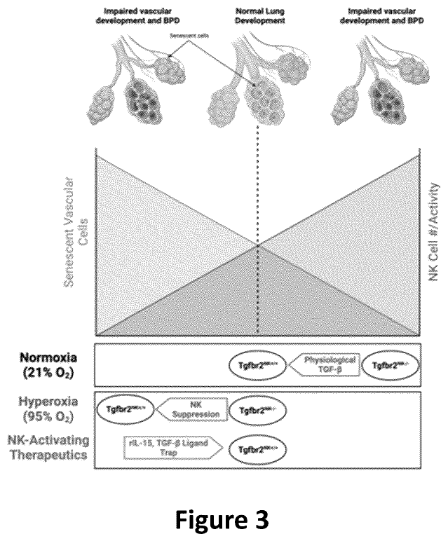

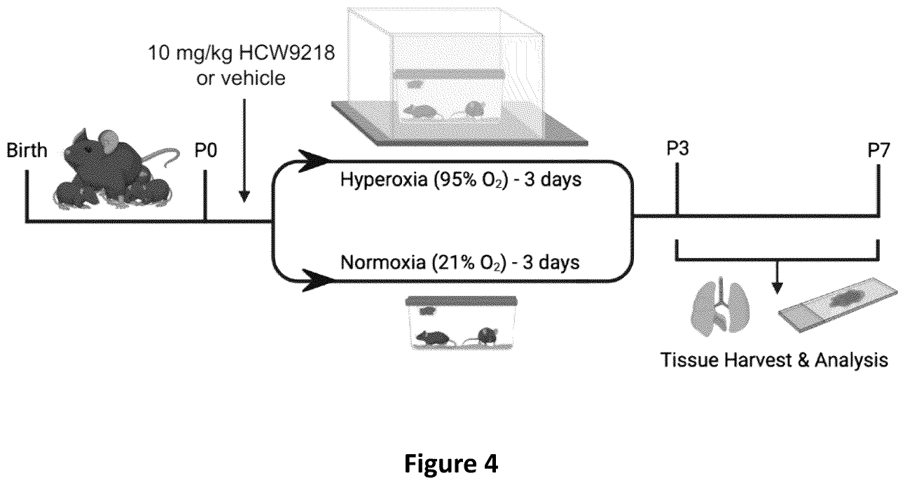

TGFRt15-TGFRs, an NK cell-targeted protein therapeutic, showed to improve airway patterning and postnatal development in a mouse model of hyperoxia-induced BPD (FIG. 3). A description of the structure of TGFRt15-TGFR (HCW9218) used in these experiments is described in Example 1. A mouse model that was used involves the impairment of pulmonary vascular and airway development through the exposure of neonatal mice to hyperoxia (95% oxygen) for 72 hours, from postnatal day 0 (P0) to P3. For this model, pulmonary vascular development, senescent lung cells and natural killer cell infiltration in the lung were assessed at P3 and P7, while impaired airway development is assessed on P7 (FIG. 4).

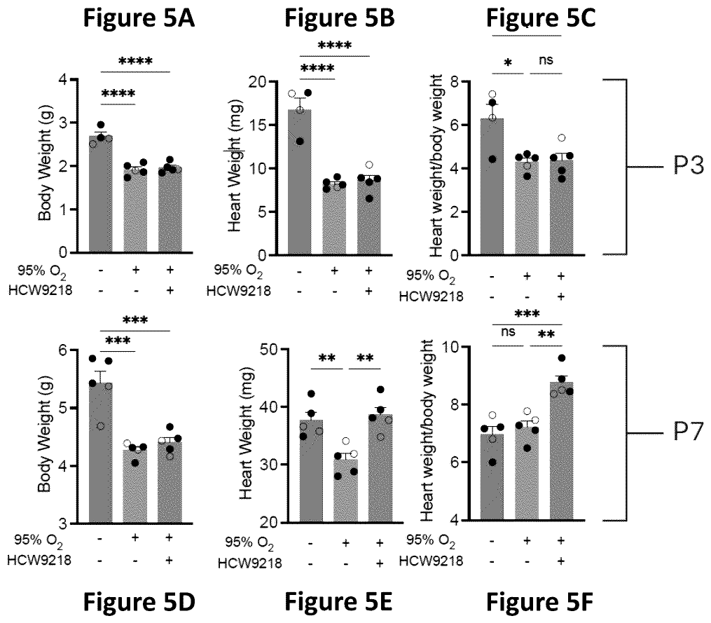

Experimental Methods: Pups born to timed pregnant dams (Charles River) were randomized into (i) Normoxia+Vehicle, (ii) Hyperoxia+Vehicle, or (iii) Hyperoxia+HCW9218 groups immediately after birth on postnatal day 0 (P0). Pups in groups (i) and (ii) received a 5 μL subcutaneous injection of PBS vehicle as a control, while pups in group (iii) received a 5 μL subcutaneous injection of 3.5 mg/mL HCW9218 in PBS (dosing of 10 mg/kg assuming a pup weight of 1.75 g) on P0. Pups and their dams were then placed immediately in hyperoxia (95% oxygen) or normoxia (21% oxygen) for 72 hours, from P0 to P3. Dams were switched between normoxia and hyperoxia every 24 hours to preserve maternal health. Cages were removed from the hyperoxic chamber on P3 and pups were sacrificed on P3 or P7. Total body weight and heart weight were quantified at the time of sacrifice (FIGS. 5A-5F).

For sacrifice on P3, the pulmonary circulation was flushed with PBS/Heparin via the right ventricle. Lungs were inflated with PBS and frozen in OCT for assessment of β-galactosidase activity, or fixed for 48 hours in 2% PFA prior to cryoprotection and embedding in OCT for the quantification of lung vascular density. For sacrifice on P7, the pulmonary circulation was flushed with PBS/Heparin via the right ventricle and lungs were inflated with 2% PFA prior to fixation for 48 hours in 2% PFA. After fixation, lungs were embedded in OCT for the quantification of lung vascular density, or embedded in paraffin for the assessment of airway structure.

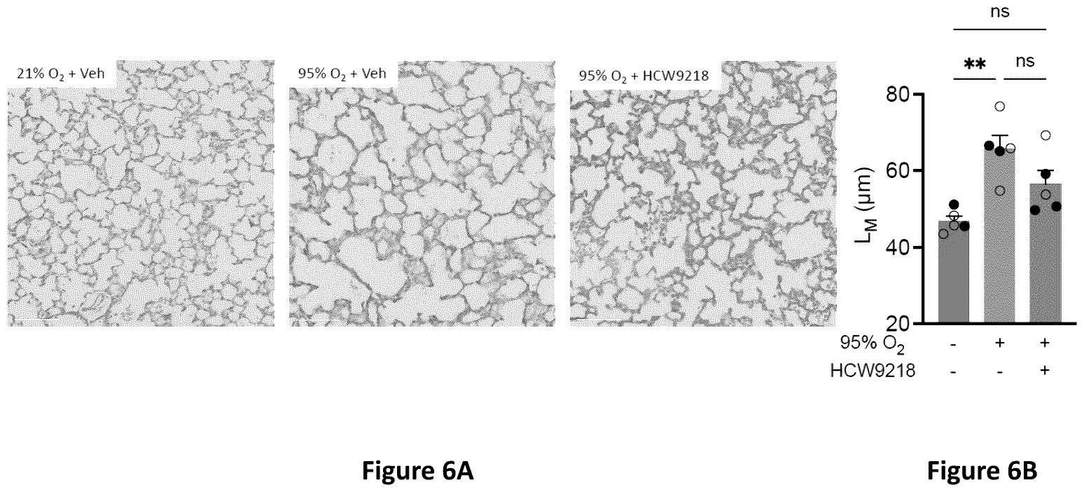

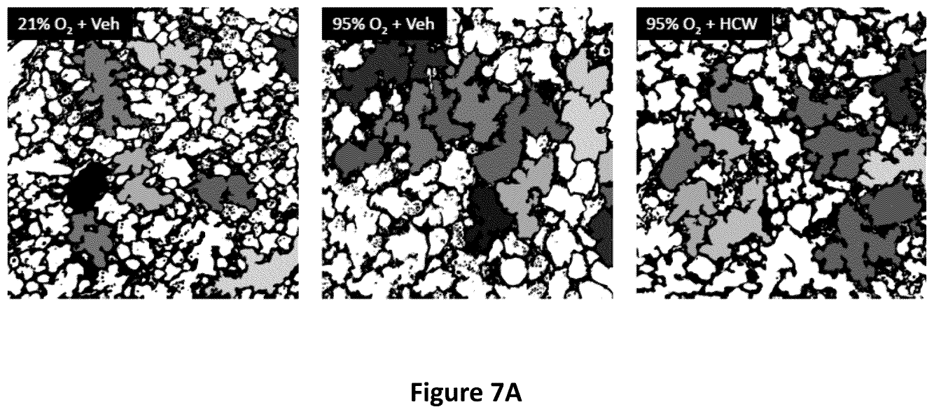

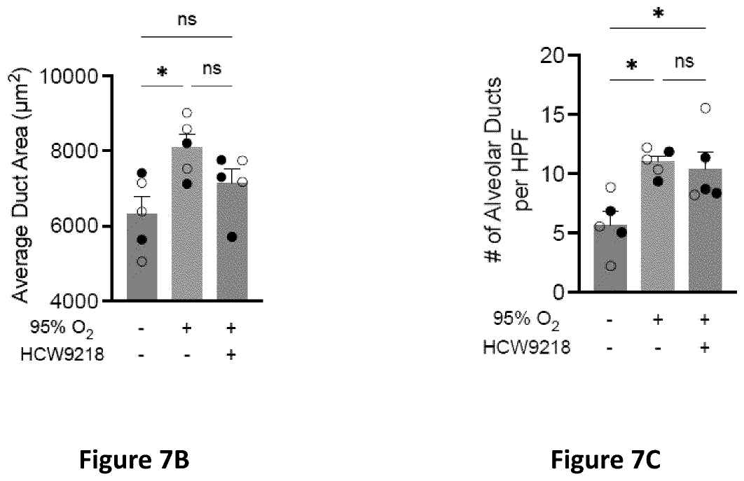

Assessment of airway structure: Alveolar density and alveolar ducts were assessed on H&E-stained lung sections (FIG. 6A). Alveolar density was assessed as a measure of mean linear intercept (LM) (FIG. 6B). Briefly, lines of known length (μm) were drawn on images of distal lung tissue, avoiding terminal respiratory bronchioles/major airways/vascular structures. MLI was calculated by dividing the line length by the number of times this line intercepted an alveolar space. Six high-powered 20× fields (HPF) were taken for each animal, and two non-overlapping measurements were taken per HPF. Alveolar ducts were assessed by the automated quantification of duct number and mean area using an ImageJ script. This script makes use of FIJI's particle analysis module, in tandem with basic preprocessing steps, to output a masked image for which large respiratory zone airways are quantified (FIGS. 7A-7C).

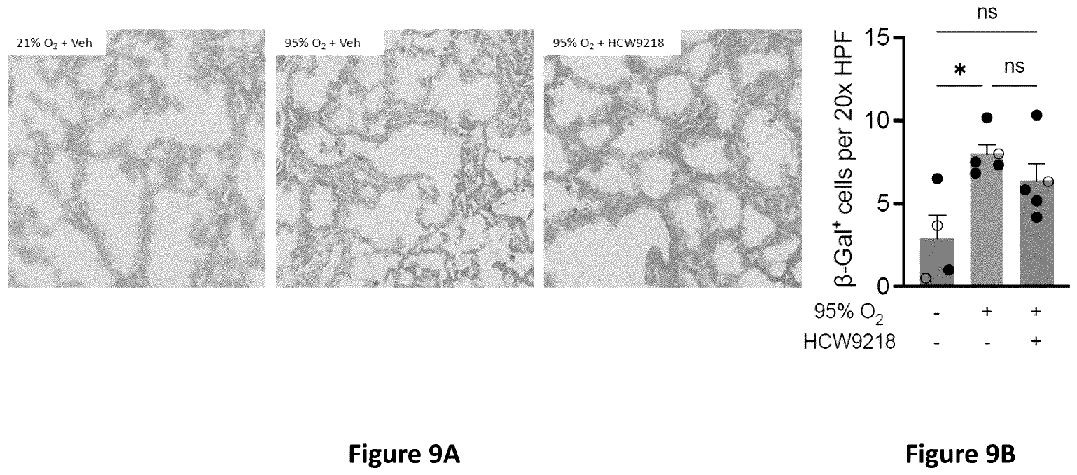

Senescence-associated β-Galactosidase staining: Briefly, fresh-frozen tissue blocks were sectioned at 10 μm. Sections were fixed with 0.2% glutaraldehyde in PBS for 10 minutes on ice and were incubated overnight in β-Galactosidase staining solution (5 mM Potassium Ferricyanide, 5 mM Potassium Ferrocyanide, 150 mM NaCl, 2 mM MgCl2 Hexahydrate, 1 mg/mL X-Gal, 1× Citric Acid/Sodium Phosphate (pH 6)) at 37° C. in darkness. A post-stain fix was performed (4% PFA, 10 minutes), and sections were counterstained with Nuclear Fast Red. Slides were dehydrated, mounted, and imaged via brightfield microscopy (FIGS. 9A-9B).

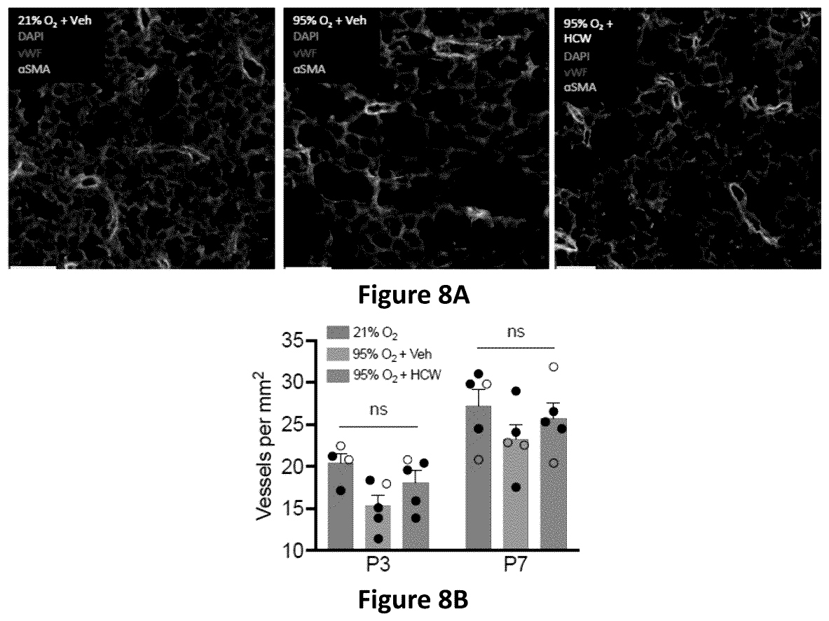

Assessment of lung vascular density: Fixed-frozen lung sections were stained with antibodies towards α-smooth muscle actin (α-SMA, clone 1A4, Invitrogen, Mouse IgG Monoclonal) and von Willebrand Factor (vWF, Cedarlane, Sheep IgG Polyclonal), followed by the appropriate fluorescently-conjugated secondary antibodies. Sections were mounted using ProLong Diamond Antifade Mountant with DAPI (ThermoFisher) to stain nuclei prior to imaging (FIG. 8A). Vascular density was taken as the average number of vessels per HPF, with 6 HPFs per animal. Measurements were scaled to vessels per mm² (FIG. 8B).

The data described above indicate that HCW9218 can be used to effectively treat bronchopulmonary dysplasia in a subject.

1. A method of treating bronchopulmonary dysplasia (BPD) in a subject, the method comprising administering to the subject a therapeutically effective amount of a multi-chain chimeric polypeptide, wherein the multi-chain chimeric polypeptide comprises:
(a) a first chimeric polypeptide comprising:
(i) a first target-binding domain;
(ii) a soluble tissue factor domain comprising a sequence that is at least 80% identical to SEQ ID NO: 1; and
(iii) a first domain of a pair of affinity domains comprising a sequence that is at least 80% identical to SEQ ID NO: 45;
(b) a second chimeric polypeptide comprising:
(i) a second domain of a pair of affinity domains comprising a sequence that is at least 80% identical to SEQ ID NO: 43; and
(ii) a second target-binding domain,
wherein:
the first chimeric polypeptide and the second chimeric polypeptide associate through the binding of the first domain and the second domain of the pair of affinity domains; and
the first target-binding domain and the second target-binding domain each comprise a soluble TGF-β receptor II (TGF-βRII) and each comprise a first sequence that is at least 80% identical to SEQ ID NO: 2 and a second sequence that is at least 80% identical to SEQ ID NO: 2.

2. The method of claim 1, wherein the first target-binding domain and the soluble tissue factor domain directly abut each other in the first chimeric polypeptide.

3. The method of claim 1, wherein the first chimeric polypeptide further comprises a linker sequence between the first target-binding domain and the soluble tissue factor domain in the first chimeric polypeptide.

4. The method of claim 1, wherein the soluble tissue factor domain and the first domain of the pair of affinity domains directly abut each other in the first chimeric polypeptide.

5. The method of claim 1, wherein the first chimeric polypeptide further comprises a linker sequence between the soluble tissue factor domain and the first domain of the pair of affinity domains in the first chimeric polypeptide.

6. The method of claim 1, wherein the second domain of the pair of affinity domains and the second target-binding domain directly abut each other in the second chimeric polypeptide.

7. The method of claim 1, wherein the second chimeric polypeptide further comprises a linker sequence between the second domain of the pair of affinity domains and the second target-binding domain in the second chimeric polypeptide.

8-12. (canceled)

13. The method of claim 1, wherein the first chimeric polypeptide further comprises one or more additional target-binding domain(s).

14. The method of claim 1, wherein the second chimeric polypeptide further comprises one or more additional target-binding domains.

15. The method of claim 1, wherein the soluble tissue factor domain is a soluble human tissue factor domain.

16-18. (canceled)

19. The method of claim 1, wherein the first chimeric polypeptide and/or the second chimeric polypeptide further comprises a signal sequence at its N-terminal end.

20-23. (canceled)

24. The method of claim 1, wherein:
the first target-binding domain comprises a sequence that is at least 80% identical to SEQ ID NO: 6; and
the second target-binding domain comprises a sequence that is at least 80% identical to SEQ ID NO: 6.

25. The method of claim 24, wherein:
the first target-binding domain comprises a sequence that is at least 90% identical to SEQ ID NO: 6;
the soluble tissue factor domain comprises a sequence that is at least 90% identical to SEQ ID NO: 1;
the first domain of the pair of affinity domains comprises a sequence that is at least 90% identical to SEQ ID NO: 45;
the second target-binding domain comprises a sequence that is at least 90% identical to SEQ ID NO: 6; and
the second domain of the pair of affinity domains comprises a sequence that is at least 90% identical to SEQ ID NO: 43.

26. The method of claim 25, wherein:
the first target-binding domain comprises a sequence of SEQ ID NO: 6,
the soluble tissue factor domain comprises a sequence of SEQ ID NO: 1;
the first domain of the pair of affinity domains comprises a sequence of SEQ ID NO: 45;
the second target-binding domain comprises a sequence of SEQ ID NO: 6; and
the second domain of the pair of affinity domains comprises a sequence of SEQ ID NO: 43.

27. The method of claim 1, wherein:
the first chimeric polypeptide comprises a sequence that is at least 80% identical to SEQ ID NO: 8; and
the second chimeric polypeptide comprises a sequence that is at least 80% identical to SEQ ID NO: 16.

28. The method of claim 27, wherein:
the first chimeric polypeptide comprises a sequence that is at least 90% identical to SEQ ID NO: 8; and
the second chimeric polypeptide comprises a sequence that is at least 90% identical to SEQ ID NO: 16.

29. The method of claim 28, wherein:
the first chimeric polypeptide comprises a sequence of SEQ ID NO: 8 and
the second chimeric polypeptide comprises a sequence of SEQ ID NO: 16.

30. (canceled)

31. The method of claim 28, wherein:
the first chimeric polypeptide comprises a sequence of SEQ ID NO: 12; and
the second chimeric polypeptide comprises a sequence of SEQ ID NO: 16.

32-43. (canceled)

44. The method of claim 1, wherein the subject has been identified or diagnosed as having bronchopulmonary dysplasia (BPD).

45. A method of treating bronchopulmonary dysplasia (BPD) in a subject, the method comprising administering to the subject a therapeutically effective amount of a multi-chain chimeric polypeptide, wherein the multi-chain chimeric polypeptide comprises:
(a) a first chimeric polypeptide comprising:
(i) a first target-binding domain comprising:
a first sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; and
a second sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine;
(ii) a soluble tissue factor domain comprises a sequence that is at least 80% identical to SEQ ID NO: 1; and
(iii) a first domain of a pair of affinity domains comprising a sequence that is at least 80% identical to SEQ ID NO: 45; and
(b) a second chimeric polypeptide comprising:
(i) a second domain of a pair of affinity domains comprising a sequence that is at least 80% identical to SEQ ID NO: 43; and
(ii) a second target-binding domain comprising:
a first sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine; and
a second sequence that is at least 80% identical to SEQ ID NO: 20, wherein one or both of (A) the amino acid at position 32 in SEQ ID NO: 20 is asparagine and (B) the amino acid at position 119 in SEQ ID NO: 20 is alanine,
wherein:
the first chimeric polypeptide and the second chimeric polypeptide associate through the binding of the first domain and the second domain of the pair of affinity domains; and
the first target-binding domain and the second target-binding domain each comprise a soluble TGF-β receptor II (TGF-αRII).

46-94. (canceled)


SEQUENCE LISTING (56 sequences)

SEQ ID NO: 1 - protein, 219 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 2 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 3 - DNA, 408 bp, Homo sapiens
  source 1..408: mol_type = genomic DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgat

SEQ ID NO: 4 - DNA, 408 bp, Homo sapiens
  source 1..408: mol_type = genomic DNA
attcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 5 - DNA, 15 bp, synthetic construct
  source 1..15: mol_type = other DNA
ggggsggggsggggs

SEQ ID NO: 6 - protein, 287 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 7 - DNA, 861 bp, Homo sapiens
  source 1..861: mol_type = genomic DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 8 - protein, 620 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 9 - DNA, 1860 bp, synthetic construct
  source 1..1860: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 10 - protein, 638 aa, synthetic construct
MKWVTFISLLFLFSSAYSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - DNA, 1914 bp, synthetic construct
  source 1..1914: mol_type = other DNA
atgaagtgggtgaccttcatcagcctgctgttcctgttctccagcgcctactccatccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 12 - protein, 620 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - DNA, 1860 bp, synthetic construct
  source 1..1860: mol_type = other DNA
atcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgactcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaaactgggtgaatgtaataagtaatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 14 - protein, 637 aa, synthetic construct
MGVKVLFALICIAVAEAIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 15 - DNA, 1911 bp, synthetic construct
  source 1..1911: mol_type = other DNA
atgggagtgaaagttctttttgcccttatttgtattgctgtggccgaggccatcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgactcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaaactgggtgaatgtaataagtaatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 16 - protein, 352 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 17 - DNA, 1056 bp, synthetic construct
  source 1..1056: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 18 - protein, 370 aa, synthetic construct
MKWVTFISLLFLFSSAYSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 19 - DNA, 1110 bp, synthetic construct
  source 1..1110: mol_type = other DNA
atgaagtgggtgaccttcatcagcctgctgttcctgttctccagcgcctactccatccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 20 - protein, 136 aa, synthetic construct
IPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPD

SEQ ID NO: 21 - DNA, 408 bp, synthetic construct
  source 1..408: mol_type = other DNA
atccccccccatgtgcaaaagagcgtgaacaacgatatgatcgtgaccgacaacaacggcgccgtgaagtttccccagctctgcaagttctgcaacgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgcctgtaacgacaacatcatcttcagcgaagagtacaacaccagcaaccctgat

SEQ ID NO: 22 - DNA, 408 bp, synthetic construct
  source 1..408: mol_type = other DNA
attcctccccacgtgcagaagagcgtgaataatgacatgatcgtgaccgataacaatggcgccgtgaaatttccccagctgtgcaaattctgcaatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgcttgcaacgacaatatcatctttagcgaggaatacaataccagcaaccccgac

SEQ ID NO: 23 - DNA, 408 bp, synthetic construct
  source 1..408: mol_type = other DNA
atcccacctcacgtgcagaaaagcgtcaataatgacatgatcgtgactgacaataacggcgccgtcaagtttccacagctgtgtaagttctgcaacgtgagattttccacatgcgacaaccagaagagctgtatgagcaactgcagcatcacaagcatctgcgagaagccacaagaggtctgcgtggccgtctggagaaagaacgacgagaacatcactctcgagactgtgtgtcacgaccctaagctcccataccatgacttcatcctcgaggatgctgcctcccctaaatgcattatgaaggagaaaaaaaagcccggcgagacattctttatgtgcagctgctcctccgacgcctgcaacgacaacatcatctttagcgaagaatacaacactagcaacccagat

SEQ ID NO: 24 - DNA, 408 bp, synthetic construct
  source 1..408: mol_type = other DNA
atcccaccacacgtgcagaagtccgtcaacaacgacatgattgtgactgacaacaacggcgccgtgaagttcccacaactctgcaagttttgcaatgtgaggttctccacatgtgacaaccagaaaagctgcatgtccaactgctccatcactagcatctgtgagaaacctcaagaggtctgtgtggctgtgtggaggaagaacgatgagaacatcacactcgagacagtctgccacgacccaaagctgccatatcacgacttcattctcgaggacgccgccagccctaagtgcatcatgaaggagaagaagaagcccggcgagacatttttcatgtgtagctgcagctccgatgcctgtaacgacaatattatctttagcgaggagtataacacatccaatccagac

SEQ ID NO: 25 - protein, 287 aa, synthetic construct
IPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPDGGGGSGGGGSGGGGSIPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPD

SEQ ID NO: 26 - DNA, 861 bp, synthetic construct
  source 1..861: mol_type = other DNA
atccccccccatgtgcaaaagagcgtgaacaacgatatgatcgtgaccgacaacaacggcgccgtgaagtttccccagctctgcaagttctgcaacgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgcctgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcaatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgcttgcaacgacaatatcatctttagcgaggaatacaataccagcaaccccgac

SEQ ID NO: 27 - DNA, 861 bp, synthetic construct
  source 1..861: mol_type = other DNA
atcccacctcacgtgcagaaaagcgtcaataatgacatgatcgtgactgacaataacggcgccgtcaagtttccacagctgtgtaagttctgcaacgtgagattttccacatgcgacaaccagaagagctgtatgagcaactgcagcatcacaagcatctgcgagaagccacaagaggtctgcgtggccgtctggagaaagaacgacgagaacatcactctcgagactgtgtgtcacgaccctaagctcccataccatgacttcatcctcgaggatgctgcctcccctaaatgcattatgaaggagaaaaaaaagcccggcgagacattctttatgtgcagctgctcctccgacgcctgcaacgacaacatcatctttagcgaagaatacaacactagcaacccagatggcggcggaggatccggaggaggaggctccggaggcggaggcagcatcccaccacacgtgcagaagtccgtcaacaacgacatgattgtgactgacaacaacggcgccgtgaagttcccacaactctgcaagttttgcaatgtgaggttctccacatgtgacaaccagaaaagctgcatgtccaactgctccatcactagcatctgtgagaaacctcaagaggtctgtgtggctgtgtggaggaagaacgatgagaacatcacactcgagacagtctgccacgacccaaagctgccatatcacgacttcattctcgaggacgccgccagccctaagtgcatcatgaaggagaagaagaagcccggcgagacatttttcatgtgtagctgcagctccgatgcctgtaacgacaatattatctttagcgaggagtataacacatccaatccagac

SEQ ID NO: 28 - protein, 620 aa, synthetic construct
IPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPDGGGGSGGGGSGGGGSIPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 29 - DNA, 1860 bp, synthetic construct
  source 1..1860: mol_type = other DNA
atccccccccatgtgcaaaagagcgtgaacaacgatatgatcgtgaccgacaacaacggcgccgtgaagtttccccagctctgcaagttctgcaacgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgcctgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcaatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgcttgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 30 - protein, 638 aa, synthetic construct
MKWVTFISLLFLFSSAYSIPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPDGGGGSGGGGSGGGGSIPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 31 - DNA, 1914 bp, synthetic construct
  source 1..1914: mol_type = other DNA
atgaagtgggtgaccttcatcagcctgctgttcctgttctccagcgcctactccatccccccccatgtgcaaaagagcgtgaacaacgatatgatcgtgaccgacaacaacggcgccgtgaagtttccccagctctgcaagttctgcaacgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgcctgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcaatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgcttgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 32 - protein, 352 aa, synthetic construct
IPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPDGGGGSGGGGSGGGGSIPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPDITCPPPMSVEHADIWVKSYSLYSRERYICNSGFKRKAGTSSLTECVLNKATNVAHWTTPSLKCIR

SEQ ID NO: 33 - DNA, 1056 bp, synthetic construct
  source 1..1056: mol_type = other DNA
atcccacctcacgtgcagaaaagcgtcaataatgacatgatcgtgactgacaataacggcgccgtcaagtttccacagctgtgtaagttctgcaacgtgagattttccacatgcgacaaccagaagagctgtatgagcaactgcagcatcacaagcatctgcgagaagccacaagaggtctgcgtggccgtctggagaaagaacgacgagaacatcactctcgagactgtgtgtcacgaccctaagctcccataccatgacttcatcctcgaggatgctgcctcccctaaatgcattatgaaggagaaaaaaaagcccggcgagacattctttatgtgcagctgctcctccgacgcctgcaacgacaacatcatctttagcgaagaatacaacactagcaacccagatggcggcggaggatccggaggaggaggctccggaggcggaggcagcatcccaccacacgtgcagaagtccgtcaacaacgacatgattgtgactgacaacaacggcgccgtgaagttcccacaactctgcaagttttgcaatgtgaggttctccacatgtgacaaccagaaaagctgcatgtccaactgctccatcactagcatctgtgagaaacctcaagaggtctgtgtggctgtgtggaggaagaacgatgagaacatcacactcgagacagtctgccacgacccaaagctgccatatcacgacttcattctcgaggacgccgccagccctaagtgcatcatgaaggagaagaagaagcccggcgagacatttttcatgtgtagctgcagctccgatgcctgtaacgacaatattatctttagcgaggagtataacacatccaatccagacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 34 - protein, 370 aa, synthetic construct
MKWVTFISLLFLFSSAYSIPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPDGGGGSGGGGSGGGGSIPPHVQKSVNNDMIVTDNNGAVKFPQLCKFCNVRFSTCDNQKSCMSNCSITSICEKPQEVCVAVWRKNDENITLETVCHDPKLPYHDFILEDAASPKCIMKEKKKPGETFFMCSCSSDACNDNIIFSEEYNTSNPDITCPPPMSVEHADIWVKSYSLYSRERYICNSGFKRKAGTSSLTECVLNKATNVAHWTTPSLKCIR

SEQ ID NO: 35 - DNA, 1110 bp, synthetic construct
  source 1..1110: mol_type = other DNA
atgaagtgggtgaccttcatcagcctgctgttcctgttctccagcgcctactccatcccacctcacgtgcagaaaagcgtcaataatgacatgatcgtgactgacaataacggcgccgtcaagtttccacagctgtgtaagttctgcaacgtgagattttccacatgcgacaaccagaagagctgtatgagcaactgcagcatcacaagcatctgcgagaagccacaagaggtctgcgtggccgtctggagaaagaacgacgagaacatcactctcgagactgtgtgtcacgaccctaagctcccataccatgacttcatcctcgaggatgctgcctcccctaaatgcattatgaaggagaaaaaaaagcccggcgagacattctttatgtgcagctgctcctccgacgcctgcaacgacaacatcatctttagcgaagaatacaacactagcaacccagatggcggcggaggatccggaggaggaggctccggaggcggaggcagcatcccaccacacgtgcagaagtccgtcaacaacgacatgattgtgactgacaacaacggcgccgtgaagttcccacaactctgcaagttttgcaatgtgaggttctccacatgtgacaaccagaaaagctgcatgtccaactgctccatcactagcatctgtgagaaacctcaagaggtctgtgtggctgtgtggaggaagaacgatgagaacatcacactcgagacagtctgccacgacccaaagctgccatatcacgacttcattctcgaggacgccgccagccctaagtgcatcatgaaggagaagaagaagcccggcgagacatttttcatgtgtagctgcagctccgatgcctgtaacgacaatattatctttagcgaggagtataacacatccaatccagacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 36 - DNA, 657 bp, synthetic construct
  source 1..657: mol_type = other DNA
agcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggag

SEQ ID NO: 37 - protein, 219 aa, Homo sapiens
SGTTNTVAAYNLTWKSTNFATALEWEPKPVNQVYTVQISTKSGDWKSKCFYTTDTECALTDEIVKDVKQTYLARVFSYPAGNVESTGSAGEPLYENSPEFTPYLETNLGQPTIQSFEQVGTKVNVTVEDERTLVARNNTALSLRDVFGKDLIYTLYYWKSSSSGKKTAKTNTNEFLIDVDKGENYCFSVQAVIPSRTVNRKSTDSPVECMGQEKGEFRE

SEQ ID NO: 38 - protein, 219 aa, Homo sapiens
SGTTNTVAAYNLTWKSTNFATALEWEPKPVNQVYTVQISTKSGDAKSKCFYTTDTECALTDEIVKDVKQTYLARVFSYPAGNVESTGSAGEPLAENSPEFTPYLETNLGQPTIQSFEQVGTKVNVTVEDERTLVARNNTALSLRDVFGKDLIYTLYYWKSSSSGKKTAKTNTNEFLIDVDKGENYCFSVQAVIPSRTVNRKSTDSPVECMGQEKGEFRE

SEQ ID NO: 39 - protein, 223 aa, Mus musculus
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGE

SEQ ID NO: 40 - protein, 224 aa, Rattus rattus
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKIGQPVIQKYEQGGTKLKVTVKDSFTLVRKNGTFLTLRQVFGNDLGYILTYRKDSSTGRKTNTTHTNEFLIDVEKGVSYCFFAQAVIFSRKTNHKSPESITKCTEQWKSVLGE

SEQ ID NO: 41 - DNA, 45 bp, synthetic construct
  source 1..45: mol_type = other DNA
ggcggtggaggatccggaggaggtggctccggcggcggaggatct

SEQ ID NO: 42 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 43 - DNA, 195 bp, Homo sapiens
  source 1..195: mol_type = genomic DNA
attacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 44 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 45 - DNA, 342 bp, Homo sapiens
  source 1..342: mol_type = genomic DNA
aactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 46 - protein, 114 aa, Homo sapiens
NWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 47 - DNA, 342 bp, Homo sapiens
  source 1..342: mol_type = genomic DNA
aactgggtgaatgtaataagtaatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 48 - protein, 18 aa, synthetic construct
MKWVTFISLLFLFSSAYS

SEQ ID NO: 49 - DNA, 54 bp, synthetic construct
  source 1..54: mol_type = other DNA
atgaaatgggtgacctttatttctttactgttcctctttagcagcgcctactcc

SEQ ID NO: 50 - DNA, 54 bp, synthetic construct
  source 1..54: mol_type = other DNA
atgaagtgggtcacatttatctctttactgttcctcttctccagcgcctacagc

SEQ ID NO: 51 - DNA, 54 bp, synthetic construct; identical to SEQ ID NO: 49
  source 1..54: mol_type = other DNA

SEQ ID NO: 52 - protein, 16 aa, synthetic construct
MKCLLYLAFLFLGVNC

SEQ ID NO: 53 - protein, 58 aa, synthetic construct
MGQIVTMFEALPHIIDEVINIVIIVLIIITSIKAVYNFATCGILALVSFLFLAGRSCG

SEQ ID NO: 54 - protein, 97 aa, synthetic construct
MPNHQSGSPTGSSDLLLSGKKQRPHLALRRKRRREMRKINRKVRRMNLAPIKEKTAWQHLQALISEAEEVLKTSQTPQNSLTLFLALLSVLGPPVTG

SEQ ID NO: 55 - protein, 30 aa, synthetic construct
MDSKGSSQKGSRLLLLLVVSNLLLCQGVVS

SEQ ID NO: 56 - protein, 16 aa, synthetic construct
MKCLLYLAFLFLGVNC